(19) United States
(12) Patent Application Publication
(10) Pub. No.: US 2019/0336678 A1
(43) Pub. Date: Nov. 7, 2019

(54) METHOD AND APPARATUS FOR ANALYTE MEASUREMENT, DISPLAY, AND ANNOTATION

(71) Applicant: OptiScan Biomedical Corporation, Hayward, CA (US)

(72) Inventor: Peter Rule, Los Altos Hills, CA (US)

(21) Appl. No.: 16/270,471

(22) Filed: Feb. 7, 2019

Related U.S. Application Data
(63) Continuation of application No. 14/208,672, filed on Mar. 13, 2014, now abandoned.
(60) Provisional application No. 61/779,008, filed on Mar. 13, 2013.

(51) Int. Cl. A61M 5/142; A61B 5/157; A61M 5/172; A61B 5/15; A61B 5/1468; A61B 5/00; A61B 5/1455; A61B 5/145

(52) U.S. Cl. CPC A61M 5/142; A61M 2205/12; A61M 5/1723; A61B 5/15003; A61B 5/150229; A61B 5/150992; A61B 5/1468; A61B 5/7475; A61B 5/1455; A61B 5/14532; A61B 5/742; A61B 5/7435; A61B 5/14503; A61B 5/14557; A61M 2205/3306; A61M 2230/201; A61B 5/7275; A61B 5/7282; A61M 2005/14208; A61M 2205/505; A61B 5/157

(57) ABSTRACT

Systems for rapid and accurate analyte measurement are described. For example, periodic glucose measurements can be achieved with high accuracy in a critical care environment by drawing blood into a device more than once per hour, analyzing blood (for example using infrared radiation through plasma). Safety and accuracy can be achieved by improved fluid control and avoidance of clotting. Data can be conveyed (e.g., displayed) to a user. A user can be allowed to annotate the data. For example, a touchscreen or other interface can allow addition of notes on a running graph of data, indicating events or other items of interest that may correspond to data readings or to particular times.

### CROSS-REFERENCE TO RELATED APPLICATIONS

This application is a continuation of U.S. application Ser. No. 14/208,672 (Atty. Docket No. OPTIS.273A), titled “METHOD AND APPARATUS FOR ANALYTE MEASUREMENT, DISPLAY, AND ANNOTATION,” filed on Mar. 13, 2014 which claims the benefit under 35 U.S.C. § 119(e) of U.S. Provisional Application No. 61/779,008 (Atty. Docket No. OPTIS.273PR), titled “METHOD AND APPARATUS FOR ANALYTE MEASUREMENT, DISPLAY, AND ANNOTATION,” filed on Mar. 13, 2013. The entire disclosure of each of the above-identified applications is incorporated by reference herein and made part of this specification.

This application also incorporates by reference herein and makes the entire disclosure of each of the following part of this specification: U.S. patent application Ser. No. 12/249,831 (Atty. Docket No. OPTIS.203A), titled “FLUID COMPONENT ANALYSIS SYSTEM AND METHOD FOR GLUCOSE MONITORING AND CONTROL,” filed on Oct. 10, 2008, which claims benefit under 35 U.S.C. § 119(e) of U.S. Provisional Application No. 60/979,044, titled “SYSTEM AND USER INTERFACE FOR INFUSION AND ANALYSIS,” filed on Oct. 10, 2007; U.S. Provisional Application No. 60/979,380, titled “FLUID COMPONENT ANALYSIS SYSTEM AND BOLUS INJECTION,” filed on Oct. 11, 2007; U.S. Provisional Application No. 61/025,260, titled “FLUID COMPONENT ANALYSIS SYSTEM AND METHOD FOR GLUCOSE MONITORING AND CONTROL,” filed on Jan. 31, 2008; U.S. Provisional Application No. 60/979,348, titled “SYSTEM AND METHOD FOR GLUCOSE MONITORING AND CONTROL,” filed on Oct. 11, 2007; U.S. Provisional Application No. 61/096,461, titled “ANALYTE MONITORING SYSTEM ANT) TREATMENT DOSING PROTOCOL,” filed on Sep. 12, 2008; U.S. Provisional Application No. 61/099,491, titled “AN ANALYTE MONITORING SYSTEM INCLUDING A TREATMENT DOSING ASSISTANT,” filed on Sep. 23, 2008; and U.S. application Ser. No. 12/559,328, titled “FLUID COMPONENT ANALYSIS SYSTEM AND METHOD FOR GLUCOSE MONITORING ANT) CONTROL.,” filed Sep. 14, 2009 (Atty, Docket No. OPTIS.247A), the entire disclosures of which are incorporated herein by reference, for all purposes.

### BACKGROUND

### Field

Some embodiments of the disclosure relate generally to methods and devices for determining a concentration of an analyte in a sample, such as an analyte in a sample of bodily fluid, as well as methods and devices which can be used to support the making of such determinations. Some embodiments of the disclosure relate to a method for determining a concentration of an analyte in a sample, determining a dose of an infusion fluid and displaying the determined concentration and/or the dose on a graphic user interface.

### Description of Related Art

It is advantageous to measure the levels of certain analytes, such as glucose, in a bodily fluid, such as blood. This can be done, for example, in a hospital or clinical setting when there is a risk that the levels of certain analytes may move outside a desired range, which in turn can jeopardize the health of a patient. Currently known systems for analyte monitoring in a hospital or clinical setting may suffer from various drawbacks.

### SUMMARY

Example embodiments described herein have several features, no single one of which is indispensable or solely responsible for their desirable attributes. Without limiting the scope of the claims, some of the advantageous features will now be summarized.

Various embodiments of a method are disclosed that includes accessing one or more of calibration sets, each calibration set usable to estimate an analyte concentration for a sample. A measurement of the sample is then accessed, and the method determines, for each of the one or more calibration sets, whether the calibration set is eligible to estimate analyte concentration for the sample. An estimated analyte concentration is then determined based at least partly on the measurement of the sample and the calibration sets determined to be eligible. In some embodiments, a plurality of calibration sets are used. In some embodiments, if none of the one or more calibration sets is eligible, the method does not determine the estimated analyte concentration, and the method may return a no read indication to a display system. In some embodiments, the method further includes processing the measurement of the sample to reduce or minimize effects on the estimate of concentration of the analyte caused be one or more interferents.

In one embodiment, a method for estimating a concentration of an analyte in a sample is disclosed. The method comprises receiving one or more calibration sets, each usable to estimate an analyte concentration for a sample, accessing a measurement of the sample, and determining, for each of the one or more calibration sets, whether the calibration set is eligible to estimate analyte concentration from the measurement of the sample. The method then determines an estimated analyte concentration for the sample based at least in part on the measurement of the sample and the calibration set(s) determined to be eligible, via execution of instructions by a processor. The sample may include at least one component of blood, and the analyte may comprise glucose. In some examples of the method, accessing a measurement of the sample may comprise accessing a raw measurement of the sample and calculating a measurement of the sample based at least in part on the raw measurement of the sample. The method may use a measurement of a sample that is a measured spectrum (e.g., a mid-infrared spectrum). The one or more calibration sets may comprise data usable to estimate an analyte concentration and a reconstructed spectrum from at least the measured spectrum. Determining if a calibration set is eligible to estimate an analyte concentration may comprise calculating, for each of the one or more calibration sets, a reconstructed spectrum based at least in part on the measured spectrum and the calibration set and comparing the reconstructed spectrum to the measured spectrum. Each of the one or more calibration sets may comprise a prediction eligibility threshold, and comparing the reconstructed spectrum to the measured spectrum may comprise calculating a distance metric based at least on the reconstructed spectrum and the measured spectrum and comparing the distance metric to the prediction eligibility threshold to determine whether the calibration set is eligible to estimate analyte concentration. In some embodiments, a plurality of calibration sets is used. In some embodiments, if none of the one or more calibration sets is eligible, the method does not determine the estimated analyte concentration, and the method may return a no read indication to a display system. In some embodiments, the method further includes processing the measurement of the sample to reduce or minimize effects on the estimate of concentration of the analyte caused be one or more interferents.

In one embodiment, a method for estimating a concentration of an analyte in a sample is disclosed. The method comprises accessing a measured spectrum of the sample, the measured spectrum comprising measurements at a plurality of wavelengths, accessing one or more calibration sets, each calibration set usable to estimate an analyte concentration and a reconstructed spectrum from at least the measured spectrum and further comprising a prediction eligibility threshold, calculating, for each of the one or more calibration sets, a reconstructed spectrum, calculating, for each of the one or more calibration sets, a distance metric by comparing the reconstructed spectrum to the measured spectrum, comparing, for each of the one or more calibration sets, the calculated distance metric to the prediction eligibility threshold to determine whether the calibration set is eligible to estimate analyte concentration, calculating, for each of the eligible calibration sets, an estimated analyte concentration based at least in part on the measured spectrum and the calibration set, calculating, for each of the eligible calibration sets, a weighting coefficient based at least in part on the calculated distance metric and the prediction eligibility threshold of the calibration set, and determining an analyte concentration for the sample by using the weighting coefficients to combine the estimated analyte concentrations for each of the eligible calibration sets. In some embodiments, a plurality of calibration sets is used. In some embodiments, if none of the one or more calibration sets is eligible, the method does not determine the estimated analyte concentration, and the method may return a no read indication to a display system. In some embodiments, the method further includes processing the measurement of the sample to reduce or minimize effects on the estimate of concentration of the analyte caused be one or more interferents.

In one embodiment, an analyte detection system is disclosed. The system comprises a sensor system configured to provide information relating to a measurement of an analyte in a sample. The system further comprises a processor system configured to execute stored program instructions such that the analyte detection system accesses one or more calibration sets, each usable to estimate an analyte concentration for a sample, accesses a measurement of the sample, determines, for each of the one or more calibration sets, whether the calibration set is eligible to estimate analyte concentration from the measurement of the sample, and determines an estimated analyte concentration for the sample based at least in part on the measurement of the sample and the calibration set(s) determined to be eligible. In some embodiments, the analyte detection system accesses a plurality of calibration sets. In some embodiments, if none of the one or more calibration sets is eligible, the system does not determine the estimated analyte concentration, and the system may return a no read indication to a display system. In some embodiments, the analyte detection system is further configured to process the measurement of the sample to reduce or minimize effects on the estimate of concentration of the analyte caused be one or more interferents.

### BRIEF DESCRIPTION OF THE DRAWINGS

The following drawings and the associated descriptions are provided to illustrate embodiments of the present disclosure and do not limit the scope of the claims.

FIG. 1 shows an embodiment of an apparatus for withdrawing and analyzing fluid samples.

FIG. 2 illustrates how various other devices can be supported on or near an embodiment of apparatus illustrated in FIG. 1.

FIG. 3 illustrates an embodiment of the apparatus in FIG. 1 configured to be connected to a patient.

FIG. 3A illustrates an embodiment of the apparatus in FIG. 1 that is not configured to be connected to a patient but which receives a fluid sample from an extracorporeal fluid container such as, for example, a test tube. This embodiment of the apparatus advantageously provides in vitro analysis of a fluid sample.

FIG. 4 is a block diagram of an embodiment of a system for withdrawing and analyzing fluid samples.

FIG. 5 schematically illustrates an embodiment of a fluid system that can be part of a system for withdrawing and analyzing fluid samples.

FIG. 6 schematically illustrates another embodiment of a fluid system that can be part of a system for withdrawing and analyzing fluid samples.

FIG. 7 is an oblique schematic depiction of an embodiment of a monitoring device.

FIG. 8 shows a cut-away side view of an embodiment of a monitoring device.

FIG. 9 shows a cut-away perspective view of an embodiment of a monitoring device.

FIG. 10 illustrates an embodiment of a removable cartridge that can interface with a monitoring device.

FIG. 11 illustrates an embodiment of a fluid routing card that can be part of the removable cartridge of FIG. 10.

FIG. 12 illustrates how non-disposable actuators can interface with the fluid routing card of FIG. 11.

FIG. 13 illustrates a modular pump actuator connected to a syringe housing that can form a portion of a removable cartridge.

FIG. 14 shows a rear perspective view of internal scaffolding and some pinch valve pump bodies.

FIG. 15 shows an underneath perspective view of a sample cell holder attached to a centrifuge interface, with a view of an interface with a sample injector.

FIG. 16 shows a plan view of a sample cell holder with hidden and/or non-surface portions illustrated using dashed lines.

FIG. 17 shows a top perspective view of the centrifuge interface connected to the sample holder.

FIG. 18 shows a perspective view of an example optical system.

FIG. 19 shows a filter wheel that can be part of the optical system of FIG. 18.

FIG. 20 schematically illustrates an embodiment of an optical system that comprises a spectroscopic analyzer adapted to measure spectra of a fluid sample.

FIG. 21 is a flowchart that schematically illustrates an embodiment of a method for estimating the concentration of an analyte in the presence of interferents.

FIG. 22 is a flowchart that schematically illustrates an embodiment of a method for performing a statistical comparison of the absorption spectrum of a sample with the spectrum of a sample population and combinations of individual library interferent spectra.

FIG. 23 is a flowchart that schematically illustrates an example embodiment of a method for estimating analyte concentration in the presence of the possible interferents.

FIGS. 24 and 25 schematically illustrate the visual appearance of embodiments of a user interface for a system for withdrawing and analyzing fluid samples.

FIG. 26 schematically depicts various components and/or aspects of a patient monitoring system and the relationships among the components and/or aspects.

FIG. 27 is a flowchart that schematically illustrates an embodiment of a method of providing glycemic control.

FIG. 28A shows a generalized method for allowing user annotation of data.

FIG. 28B shows an example of the generalized method of FIG. 28A.

FIG. 29A shows a graphical user interface that may be used for annotation of data.

FIG. 29B shows a graphical user interface and a user selecting data for annotation.

FIG. 29C shows a graphical user interface and an example input view.

FIG. 29D shows a graphical user interface and a user entering an annotation.

FIG. 29E shows a graphical user interface and a symbol for retrieval of the annotation.

FIG. 30 shows a loading screen of a graphical user interface of a system as described herein.

FIG. 31 shows a log in screen of a graphical user interface of a system as described herein.

FIGS. 32A-32E shows a patient selection screen of a graphical user interface of a system as described herein.

FIG. 32F shows a patient addition screen of a graphical user interface of a system as described herein.

FIG. 33-34 shows an instruction screen of a graphical user interface of a system as described herein.

FIG. 35A shows an instruction screen of a graphical user interface of a system as described herein.

FIG. 35B shows an instruction loading screen of a graphical user interface of a system as described herein.

FIG. 35C shows an instruction completion screen of a graphical user interface of a system as described herein.

FIG. 36A shows an instruction screen of a graphical user interface of a system as described herein.

FIGS. 36B-36E show screenshots of an instruction screen of a graphical user interface of a system as described herein.

FIG. 36F shows an instruction screen of a graphical user interface of a system as described herein.

FIGS. 37A-38 show screenshots of an instruction screen of a graphical user interface of a system as described herein.

FIG. 39 shows a monitoring loading screen of a graphical user interface of a system as described herein.

FIGS. 40A-41C shows a monitoring screen of a graphical user interface of a system as described herein.

FIG. 42A shows a monitoring screen of a graphical user interface of a system as described herein.

FIG. 42B shows a monitoring screen including data selected for annotation of a graphical user interface of a system as described herein.

FIG. 43 shows a monitoring screen including a menu display of a graphical user interface of a system as described herein.

FIG. 44 shows a monitoring screen including a notice bar of a graphical user interface of a system as described herein.

FIG. 45 shows a monitoring screen including a warning bar of a graphical user interface of a system as described herein.

FIG. 46 shows a monitoring screen including a warning bar of a graphical user interface of a system as described herein.

FIG. 47A shows a monitoring screen displaying a table format of a graphical user interface of a system as described herein.

FIGS. 47B-47C show screenshots of a monitoring screen displaying multiple trend indicators of a graphical user interface of a system as described herein.

FIG. 48 shows a monitoring screen displaying multiple trend indicators of a graphical user interface of a system as described herein.

FIG. 49 shows a monitoring screen displaying multiple trend indicators of a graphical user interface of a system as described herein.

These and other features will now be described with reference to the drawings summarized above. The drawings and the associated descriptions are provided to illustrate embodiments and not to limit the scope of any claim. Throughout the drawings, reference numbers may be reused to indicate correspondence between referenced elements. In addition, where applicable, the first one or two digits of a reference numeral for an element can frequently indicate the figure number in which the element first appears.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient such as, for example, in vitro techniques.

### Analyte Monitoring Apparatus

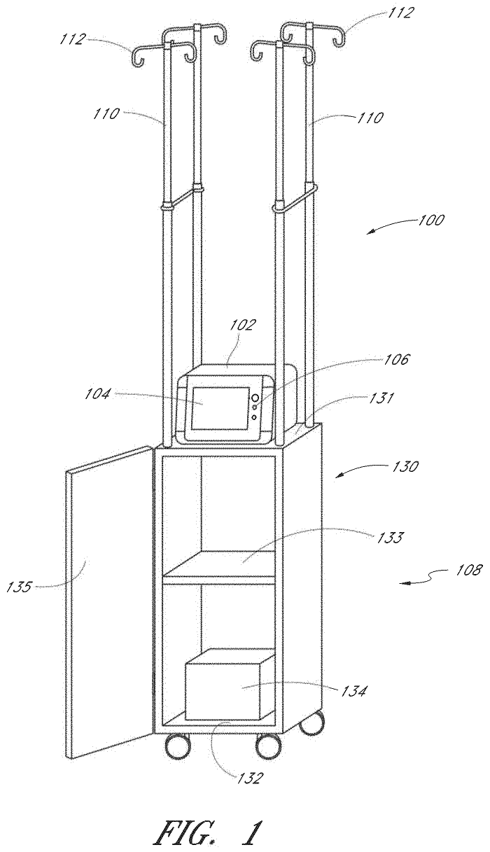

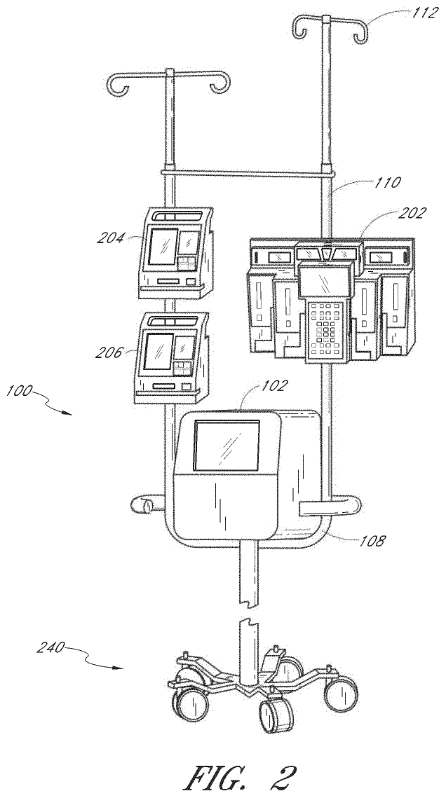

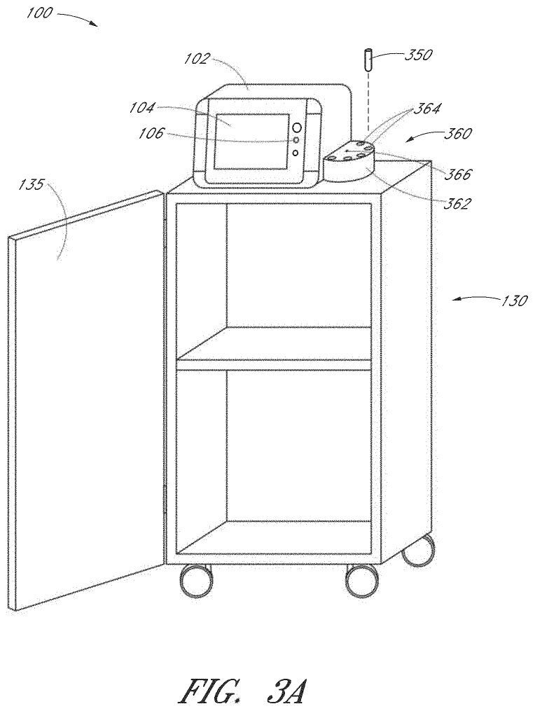

FIG. 1 shows an embodiment of an apparatus 100 for withdrawing and analyzing fluid samples. The apparatus 100 includes a monitoring device 102. In some embodiments, the monitoring device 102 can be an “OptiScanner®” monitor available from OptiScan Biomedical Corporation of Hayward, Calif. In some embodiments, the device 102 can measure one or more physiological parameters, such as the concentration of one or more substance(s) in a sample fluid. The sample fluid can be, for example, whole blood from a patient 302 (see, e.g., FIG. 3) and/or a component of whole blood such as, e.g., blood plasma. In some embodiments, the device 100 can also deliver an infusion fluid to a patient.

In the illustrated embodiment, the monitoring device 102 includes a display 104 such as, for example, a touch-sensitive liquid crystal display. The display 104 can provide an interface that includes alerts, indicators, charts, and/or soft buttons. The device 102 also can include one or more inputs and/or outputs 106 that provide connectivity and/or permit user interactivity.

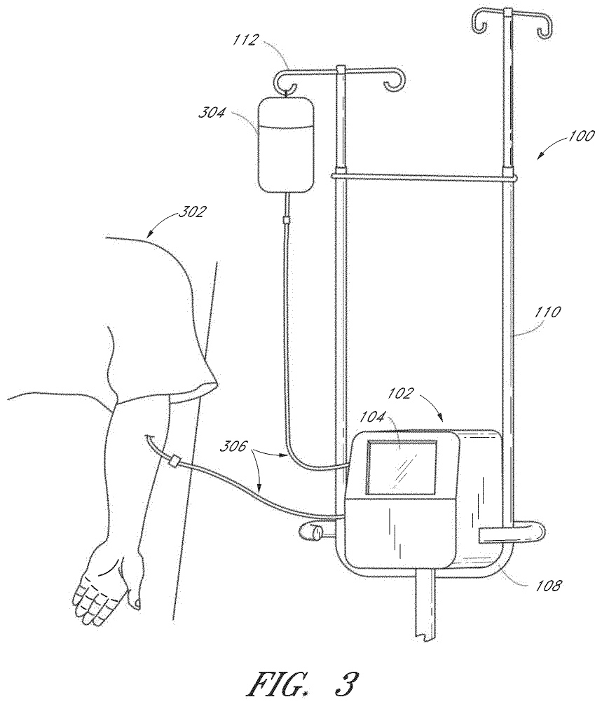

In the embodiment shown in FIG. 1, the device 102 is mounted on a stand 108. The stand 108 may comprise a cart such as, for example, a wheeled cart 130 as shown in FIG. 1. In some embodiments, the stand 108 is configured to roll on a wheeled pedestal 240 (shown in FIG. 2). The stand 108 advantageously can be easily moved and includes one or more poles 110 and/or hooks 112. The poles 110 and hooks 112 can be configured to accommodate other medical devices and/or implements, including, for example, infusion pumps, saline bags, arterial pressure sensors, other monitors and medical devices, and so forth. Some stands or carts may become unstable if intravenous (IV) bags, IV pumps, and other medical devices are hung too high on the stand or cart. In some embodiments, the apparatus 100 can be configured to have a low center of gravity, which may overcome possible instability. For example, the stand 108 can be weighted at the bottom to at least partially offset the weight of IV bags, IV pumps and medical devices that may be attached to the hooks 112 that are placed above the monitoring device 102. Adding weight toward the bottom (e.g., near the wheels) may help prevent the apparatus 100 from tipping over.

In some embodiments, the apparatus 100 includes the cart 130, which has an upper shelf 131 on which the monitoring device 102 may be placed (or attached) and a bottom shelf 132 on which a battery 134 may be placed (or attached). The battery 134 may be used as a main or backup power supply for the monitoring device 102 (which may additionally or alternatively accept electrical power from a wall socket). Two or more batteries are used in certain embodiments. The apparatus 100 may be configured so that the upper and lower shelves 131, 132 are close to ground level, and the battery provides counterweight. Other types of counterweights may be used. For example, in some embodiments, portions of the cart 130 near the floor (e.g., a lower shelf) are weighted, formed from a substantial quantity of material (e.g., thick sheets of metal), and/or formed from a relatively high-density metal (e.g., lead). In some embodiments the bottom shelf 132 is approximately 6 inches to 1 foot above ground level, and the upper shelf 131 is approximately 2 feet to 4 feet above ground level. In some embodiments the upper shelf 131 may be configured to support approximately 40 pounds (lbs.), and the bottom shelf 132 may be configured to support approximately 20 lbs. One possible advantage of embodiments having such a configuration is that IV pumps, bags containing saline, blood and/or drugs, and other medical equipment weighing approximately 60 lbs., collectively, can be hung on the hooks 112 above the shelves without making the apparatus 100 unstable. The apparatus 100 may be moved by applying a horizontal force on the apparatus 100, for example, by pushing and/or pulling the poles 110. In many cases, a user may exert force on an upper portion of the apparatus 100, for example, close to shoulder-height. By counterbalancing the weight as described above, the apparatus 100 may be moved in a reasonably stable manner.

In the illustrated embodiment, the cart 130 includes the bottom shelf 132 and an intermediate shelf 133, which are enclosed on three sides by walls and on a fourth side by a door 135. The door 135 can be opened (as shown in FIG. 1) to provide access to the shelves 132, 133. In other embodiments, the fourth side is not enclosed (e.g., the door 135 is not used). Many cart variations are possible. In some embodiments the battery 134 can be placed on the bottom shelf 134 or the intermediate shelf 133.

FIG. 2 illustrates how various other devices can be supported on or near the apparatus 100 illustrated in FIG. 1. For example, the poles 110 of the stand 108 can be configured (e.g., of sufficient size and strength) to accommodate multiple devices 202, 204, 206. In some embodiments, one or more COLLEAGUE® volumetric infusion pumps available from Baxter International Inc. of Deerfield, Ill. can be accommodated. In some embodiments, one or more Alaris® PC units available from Cardinal Health, Inc. of Dublin, Ohio can be accommodated. Furthermore, various other medical devices (including the two examples mentioned here), can be integrated with the disclosed monitoring device 102 such that multiple devices function in concert for the benefit of one or multiple patients without the devices interfering with each other.

FIG. 3 illustrates the apparatus 100 of FIG. 1 as it can be connected to a patient 302. The monitoring device 102 can be used to determine the concentration of one or more substances in a sample fluid. The sample fluid can come can come from the patient 302, as illustrated in FIG. 3, or the sample fluid can come from a fluid container, as illustrated in FIG. 3A. In some preferred embodiments, the sample fluid is whole blood.

In some embodiments (see, e.g., FIG. 3), the monitoring device 102 can also deliver an infusion fluid to the patient 302. An infusion fluid container 304 (e.g., a saline bag), which can contain infusion fluid (e.g., saline and/or medication), can be supported by the hook 112. The monitoring device 102 can be in fluid communication with both the container 304 and the sample fluid source (e.g., the patient 302), through tubes 306. The infusion fluid can comprise any combination of fluids and/or chemicals. Some advantageous examples include (but are not limited to): water, saline, dextrose, lactated Ringer's solution, drugs, and insulin.

The example monitoring device 102 schematically illustrated in FIG. 3 allows the infusion fluid to pass to the patient 302 and/or uses the infusion fluid itself (e.g., as a flushing fluid or a standard with known optical properties, as discussed further below). In some embodiments, the monitoring device 102 may not employ infusion fluid. The monitoring device 102 may thus draw samples without delivering any additional fluid to the patient 302. The monitoring device 102 can include, but is not limited to, fluid handling and analysis apparatuses, connectors, passageways, catheters, tubing, fluid control elements, valves, pumps, fluid sensors, pressure sensors, temperature sensors, hematocrit sensors, hemoglobin sensors, colorimetric sensors, gas (e.g., “bubble”) sensors, fluid conditioning elements, gas injectors, gas filters, blood plasma separators, and/or communication devices (e.g., wireless devices) to permit the transfer of information within the monitoring device 102 or between the monitoring device 102 and a network.

In some embodiments, the apparatus 100 is not connected to a patient and may receive fluid samples from a container such as a decanter, flask, beaker, tube, cartridge, test strip, etc., or any other extracorporeal fluid source. The container may include a biological fluid sample such as, e.g., a body fluid sample. For example, FIG. 3A schematically illustrates an embodiment of the monitoring device 102 that is configured to receive a fluid sample from one or more test tubes 350. This embodiment of the monitoring device 102 is configured to perform in vitro analysis of a fluid (or a fluid component) in the test tube 350. The test tube 350 may comprise a tube, vial, bottle, or other suitable container or vessel. The test tube 350 may include an opening disposed at one end of the tube through which the fluid sample may be added prior to delivery of the test tube to the monitoring device 102. In some embodiments, the test tubes 350 may also include a cover adapted to seal the opening of the tube. The cover may include an aperture configured to permit a tube, nozzle, needle, pipette, or syringe to dispense the fluid sample into the test tube 350. The test tubes 350 may comprise a material such as, for example, glass, polyethylene, or polymeric compounds. In various embodiments, the test tubes 350 may be reusable units or may be disposable, single-use units. In certain embodiments, the test tubes 350 may comprise commercially available low pressure/vacuum sample bottles, test bottles, or test tubes.

In the embodiment shown in FIG. 3A, the monitoring device 102 comprises a fluid delivery system 360 configured to receive a container (e.g., the test tube 350) containing a fluid sample and deliver the fluid sample to a fluid handling system (such as, e.g., fluid handling system 404 described below). In some embodiments, the fluid handling system delivers a portion of the fluid sample to an analyte detection system for in vitro measurement of one or more physiological parameters (e.g., an analyte concentration). Prior to measurement, the fluid handling system may, in some embodiments, separate the fluid sample into components, and a measurement may be performed on one or more of the components. For example, the fluid sample in the test tube 350 may comprise whole blood, and the fluid handling system may separate blood plasma from the sample (e.g., by filtering and/or centrifuging).

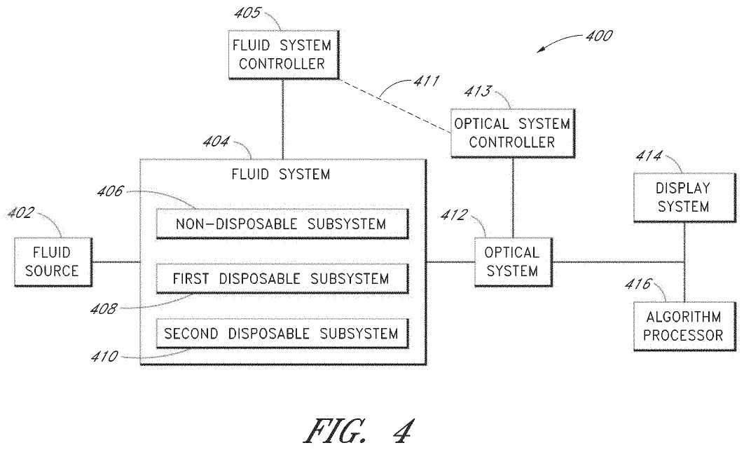

In the embodiment illustrated in FIG. 3A, the fluid delivery system 360 comprises a carousel 362 having one or more openings 364 adapted to receive the test tube 350. The carousel 362 may comprise one, two, four, six, twelve, or more openings 364. In the illustrated embodiment, the carousel 362 is configured to rotate around a central axis or spindle 366 so that a test tube 350 inserted into one of the openings 364 is delivered to the monitoring device 102. In certain embodiments, the fluid handling system of the monitoring device 102 comprises a sampling probe that is configured to collect a portion of the fluid sample from the test tube 350 (e.g., by suction or aspiration). The collected portion may then be transported in the device 102 as further described below (see, e.g., FIGS. 4-7). For example, in one embodiment suitable for use with whole blood, the collected portion of the whole blood sample is transported to a centrifuge for separation into blood plasma, a portion of the blood plasma is transported to an infrared spectroscope for measurement of one or more analytes (e.g., glucose), and the measured blood plasma is then transported to a waste container for disposal.

In other embodiments of the apparatus 100 shown in FIG. 3A, the fluid delivery system 360 may comprise a turntable, rack, or caddy adapted to receive the test tube 350. In yet other embodiments, the monitoring device 102 may comprise an inlet port adapted to receive the test tube 350. Additionally, in other embodiments, the fluid sample may be delivered to the apparatus 100 using a test cartridge, a test strip, or other suitable container. Many variations are possible.

In some embodiments, one or more components of the apparatus 100 can be located at another facility, room, or other suitable remote location. One or more components of the monitoring device 102 can communicate with one or more other components of the monitoring device 102 (or with other devices) by communication interface(s) such as, but not limited to, optical interfaces, electrical interfaces, and/or wireless interfaces. These interfaces can be part of a local network, internet, wireless network, or other suitable networks.

### System Overview

FIG. 4 is a block diagram of a system 400 for sampling and analyzing fluid samples. The monitoring device 102 can comprise such a system. The system 400 can include a fluid source 402 connected to a fluid-handling system 404. The fluid-handling system 404 includes fluid passageways and other components that direct fluid samples. Samples can be withdrawn from the fluid source 402 and analyzed by an optical system 412. The fluid-handling system 404 can be controlled by a fluid system controller 405, and the optical system 412 can be controlled by an optical system controller 413. The sampling and analysis system 400 can also include a display system 414 and an algorithm processor 416 that assist in fluid sample analysis and presentation of data.

In some embodiments, the sampling and analysis system 400 is a mobile point-of-care apparatus that monitors physiological parameters such as, for example, blood glucose concentration. Components within the system 400 that may contact fluid and/or a patient, such as tubes and connectors, can be coated with an antibacterial coating to reduce the risk of infection. Connectors between at least some components of the system 400 can include a self-sealing valve, such as a spring valve, in order to reduce the risk of contact between port openings and fluids, and to guard against fluid escaping from the system. Other components can also be included in a system for sampling and analyzing fluid in accordance with the described embodiments.

The sampling and analysis system 400 can include a fluid source 402 (or more than one fluid source) that contain(s) fluid to be sampled. The fluid-handling system 404 of the sampling and analysis system 400 is connected to, and can draw fluid from, the fluid source 402. The fluid source 402 can be, for example, a blood vessel such as a vein or an artery, a container such as a decanter, flask, beaker, tube, cartridge, test strip, etc., or any other corporeal or extracorporeal fluid source. For example, in some embodiments, the fluid source 402 may be a vein or artery in the patient 302 (see, e.g., FIG. 3). In other embodiments, the fluid source 402 may comprise an extracorporeal container 350 of fluid delivered to the system 400 for analysis (see, e.g., FIG. 3B). The fluid to be sampled can be, for example, blood, plasma, interstitial fluid, lymphatic fluid, or another fluid. In some embodiments, more than one fluid source can be present, and more than one fluid and/or type of fluid can be provided.

In some embodiments, the fluid-handling system 404 withdraws a sample of fluid from the fluid source 402 for analysis, centrifuges at least a portion of the sample, and prepares at least a portion of the sample for analysis by an optical sensor such as a spectrophotometer (which can be part of an optical system 412, for example). These functions can be controlled by a fluid system controller 405, which can also be integrated into the fluid-handling system 404. The fluid system controller 405 can also control the additional functions described below. In some embodiments, the sample can be withdrawn continuously or substantially continuously at certain time intervals with a given period. The time intervals at which the sample is withdrawn can be periodic or aperiodic and range from approximately 1 minute to approximately 15 minutes (e.g., the sample can be withdrawn at time intervals of 1 minute, 5 minutes, 10 minutes or 15 minutes). In some embodiments, the sample can be withdrawn at discrete time intervals (e.g., once every 30 minutes, once every 45 minutes or once every hour).

The duration of time over which the sample of fluid is withdrawn, referred to as “draw period”, may be set to avoid clinical drawbacks, and/or it can be varied according to a health-care provider's wishes. For example, in some embodiments, fluid may be continuously withdrawn into the sampling and analysis system 400 over a draw period lasting approximately 10 seconds to approximately 5 minutes.

In some embodiments, the amount of sample withdrawn from the fluid source 402 can be small. For example, in some embodiments, the volume of sample withdrawn from the fluid source can be between approximately 1.0 ml and approximately 10.0 ml in a draw period (e.g. 2.0 ml-6.0 ml of sample can be withdrawn in a draw period of approximately 1 minute). In some embodiments, the amount of sample withdrawn can be in the range of approximately 20 ml/day to approximately 500 ml/day. In some embodiments, the amount of sample withdrawn can be outside this range.

In some embodiments, at least a portion of the sample is returned to the fluid source 402. At least some of the sample, such as portions of the sample that are mixed with other materials or portions that are otherwise altered during the sampling and analysis process, or portions that, for any reason, are not to be returned to the fluid source 402, can also be placed in a waste bladder (not shown in FIG. 4). The waste bladder can be integrated into the fluid-handling system 404 or supplied by a user of the system 400. The fluid-handling system 404 can also be connected to a saline source, a detergent source, and/or an anticoagulant source, each of which can be supplied by a user, attached to the fluid-handling system 404 as additional fluid sources, and/or integrated into the fluid-handling system 404.

Components of the fluid-handling system 404 can be modularized into one or more non-disposable, disposable, and/or replaceable subsystems. In the embodiment shown in FIG. 4, components of the fluid-handling system 404 are separated into a non-disposable subsystem 406, a first disposable subsystem 408, and a second disposable subsystem 410.

The non-disposable subsystem 406 can include components that, while they may be replaceable or adjustable, do not generally require regular replacement during the useful lifetime of the system 400. In some embodiments, the non-disposable subsystem 406 of the fluid-handling system 404 includes one or more reusable valves and sensors. For example, the non-disposable subsystem 406 can include one or more valves (or non-disposable portions thereof), (e.g., pinch-valves, rotary valves, etc.), sensors (e.g., ultrasonic bubble sensors, non-contact pressure sensors, optical blood dilution sensors, etc.). The non-disposable subsystem 406 can also include one or more pumps (or non-disposable portions thereof). For example, some embodiments can include pumps available from Hospira. In some embodiments, the components of the non-disposable subsystem 406 are not directly exposed to fluids and/or are not readily susceptible to contamination.

The first and second disposable subsystems 408, 410 can include components that are regularly replaced under certain circumstances in order to facilitate the operation of the system 400. For example, the first disposable subsystem 408 can be replaced after a certain period of use, such as a few days, has elapsed. Replacement may be necessary, for example, when a bladder within the first disposable subsystem 408 is filled to capacity. Such replacement may mitigate fluid system performance degradation associated with and/or contamination wear on system components.

In some embodiments, the first disposable subsystem 408 includes components that may contact fluids such as patient blood, saline, flushing solutions, anticoagulants, and/or detergent solutions. For example, the first disposable subsystem 408 can include one or more tubes, fittings, cleaner pouches and/or waste bladders. The components of the first disposable subsystem 408 can be sterilized in order to decrease the risk of infection and can be configured to be easily replaceable.

In some embodiments, the second disposable subsystem 410 can be designed to be replaced under certain circumstances. For example, the second disposable subsystem 410 can be replaced when the patient being monitored by the system 400 is changed. The components of the second disposable subsystem 410 may not need replacement at the same intervals as the components of the first disposable subsystem 408. For example, the second disposable subsystem 410 can include a sample holder and/or at least some components of a centrifuge, components that may not become filled or quickly worn during operation of the system 400. Replacement of the second disposable subsystem 410 can decrease or eliminate the risk of transferring fluids from one patient to another during operation of the system 400, enhance the measurement performance of system 400, and/or reduce the risk of contamination or infection.

In some embodiments, the sample holder of the second disposable subsystem 410 receives the sample obtained from the fluid source 402 via fluid passageways of the first disposable subsystem 408. The sample holder is a container that can hold fluid for the centrifuge and can include a window to the sample for analysis by a spectrometer. In some embodiments, the sample holder includes windows that are made of a material that is substantially transparent to electromagnetic radiation in the mid-infrared range of the spectrum. For example, the sample holder windows can be made of calcium fluoride.

An injector can provide a fluid connection between the first disposable subsystem 408 and the sample holder of the second disposable subsystem 410. In some embodiments, the injector can be removed from the sample holder to allow for free spinning of the sample holder during centrifugation.

In some embodiments, the components of the sample are separated by centrifuging for a period of time before measurements are performed by the optical system 412. For example, a fluid sample (e.g., a blood sample) can be centrifuged at a relatively high speed. The sample can be spun at a certain number of revolutions per minute (RPM) for a given length of time to separate blood plasma for spectral analysis. In some embodiments, the fluid sample is spun at about 7200 RPM. In some embodiments, the sample is spun at about 5000 RPM. In some embodiments, the fluid sample is spun at about 4500 RPM. In some embodiments, the fluid sample is spun at more than one rate for successive time periods. The length of time can be approximately 5 minutes. In some embodiments, the length of time is approximately 2 minutes. Separation of a sample into the components can permit measurement of solute (e.g., glucose) concentration in plasma, for example, without interference from other blood components. This kind of post-separation measurement, (sometimes referred to as a “direct measurement”) has advantages over a solute measurement taken from whole blood because the proportions of plasma to other components need not be known or estimated in order to infer plasma glucose concentration. In some embodiments, the separated plasma can be analyzed electrically using one or more electrodes instead of, or in addition to, being analyzed optically. This analysis may occur within the same device, or within a different device. For example, in certain embodiments, an optical analysis device can separate blood into components, analyze the components, and then allow the components to be transported to another analysis device that can further analyze the components (e.g., using electrical and/or electrochemical measurements).

An anticoagulant, such as, for example, heparin can be added to the sample before centrifugation to prevent clotting. The fluid-handling system 404 can be used with a variety of anticoagulants, including anticoagulants supplied by a hospital or other user of the monitoring system 400. A detergent solution formed by mixing detergent powder from a pouch connected to the fluid-handling system 404 with saline can be used to periodically clean residual protein and other sample remnants from one or more components of the fluid-handling system 404, such as the sample holder. Sample fluid to which anticoagulant has been added and used detergent solution can be transferred into the waste bladder.

The system 400 shown in FIG. 4 includes an optical system 412 that can measure optical properties (e.g., transmission) of a fluid sample (or a portion thereof). In some embodiments, the optical system 412 measures transmission in the mid-infrared range of the spectrum. In some embodiments, the optical system 412 includes a spectrometer that measures the transmission of broadband infrared light through a portion of a sample holder filled with fluid. The spectrometer need not come into direct contact with the sample. As used herein, the term “sample holder” is a broad term that carries its ordinary meaning as an object that can provide a place for fluid. The fluid can enter the sample holder by flowing.

In some embodiments, the optical system 412 includes a filter wheel that contains one or more filters. In some embodiments, more than ten filters can be included, for example twelve or fifteen filters. In some embodiments, more than 20 filters (e.g., twenty-five filters) are mounted on the filter wheel. The optical system 412 includes a light source that passes light through a filter and the sample holder to a detector. In some embodiments, a stepper motor moves the filter wheel in order to position a selected filter in the path of the light. An optical encoder can also be used to finely position one or more filters. In some embodiments, one or more tunable filters may be used to filter light into multiple wavelengths. The one or more tunable filters may provide the multiple wavelengths of light at the same time or at different times (e.g., sequentially). The light source included in the optical system 412 may emit radiation in the ultraviolet, visible, near-infrared, mid-infrared, and/or far-infrared regions of the electromagnetic spectrum. In some embodiments, the light source can be a broadband source that emits radiation in a broad spectral region (e.g., from about 1500 nm to about 6000 nm). In other embodiments, the light source may emit radiation at certain specific wavelengths. The light source may comprise one or more light emitting diodes (LEDs) emitting radiation at one or more wavelengths in the radiation regions described herein. In other embodiments, the light source may comprise one or more laser modules emitting radiation at one or more wavelengths. The laser modules may comprise a solid state laser (e.g., a Nd:YAG laser), a semiconductor based laser (e.g., a GaAs and/or InGaAsP laser), and/or a gas laser (e.g., an Ar-ion laser). In some embodiments, the laser modules may comprise a fiber laser. The laser modules may emit radiation at certain fixed wavelengths. In some embodiments, the emission wavelength of the laser module(s) may be tunable over a wide spectral range (e.g., about 30 nm to about 100 nm). In some embodiments, the light source included in the optical system 412 may be a thermal infrared emitter. The light source can comprise a resistive heating element, which, in some embodiments, may be integrated on a thin dielectric membrane on a micro-machined silicon structure. In one embodiment the light source is generally similar to the electrical modulated thermal infrared radiation source, IRSource™, available from the Axetris Microsystems division of Leister Technologies, LLC (Itasca, Ill.).

The optical system 412 can be controlled by an optical system controller 413. The optical system controller can, in some embodiments, be integrated into the optical system 412. In some embodiments, the fluid system controller 405 and the optical system controller 413 can communicate with each other as indicated by the line 411. In some embodiments, the function of these two controllers can be integrated and a single controller can control both the fluid-handling system 404 and the optical system 412. Such an integrated control can be advantageous because the two systems are preferably integrated, and the optical system 412 is preferably configured to analyze the very same fluid handled by the fluid-handling system 404. Indeed, portions of the fluid-handling system 404 (e.g., the sample holder described above with respect to the second disposable subsystem 410 and/or at least some components of a centrifuge) can also be components of the optical system 412. Accordingly, the fluid-handling system 404 can be controlled to obtain a fluid sample for analysis by optical system 412, when the fluid sample arrives, the optical system 412 can be controlled to analyze the sample, and when the analysis is complete (or before), the fluid-handling system 404 can be controlled to return some of the sample to the fluid source 402 and/or discard some of the sample, as appropriate.

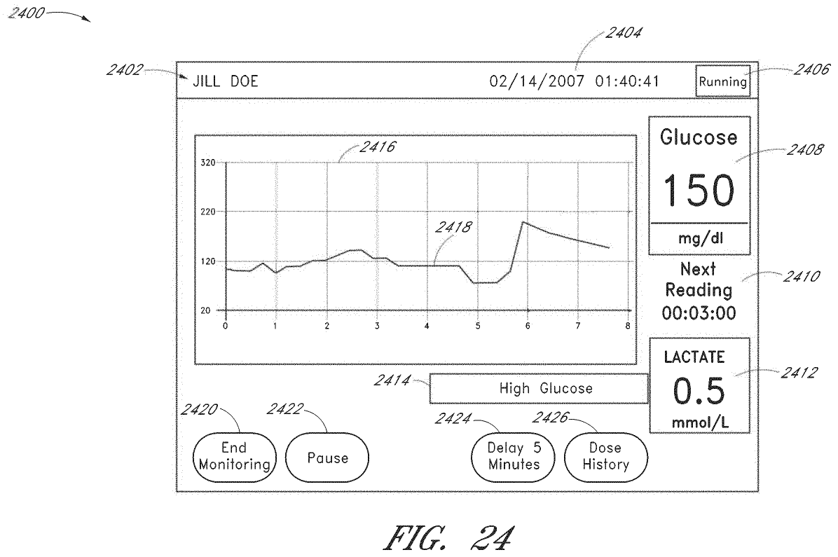

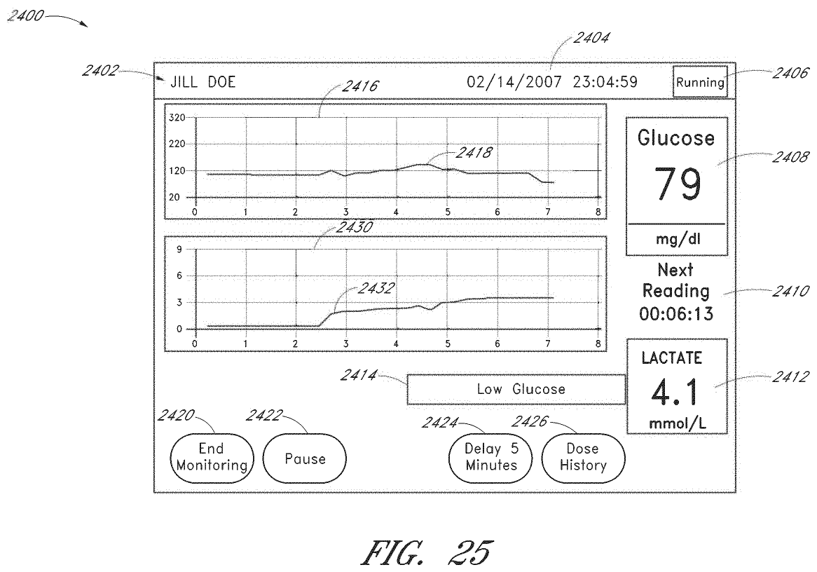

The system 400 shown in FIG. 4 includes a display system 414 that provides for communication of information to a user of the system 400. In some embodiments, the display 414 can be replaced by or supplemented with other communication devices that communicate in non-visual ways. The display system 414 can include a display processor that controls or produces an interface to communicate information to the user. The display system 414 can include a display screen. One or more parameters such as, for example, blood glucose concentration, system 400 operating parameters, and/or other operating parameters can be displayed on a monitor (not shown) associated with the system 400. An example of one way such information can be displayed is shown in FIGS. 24 and 25. In some embodiments, the display system 414 can communicate measured physiological parameters and/or operating parameters to a computer system over a communications connection.

The system 400 shown in FIG. 4 includes an algorithm processor 416 that can receive spectral information, such as optical density (OD) values (or other analog or digital optical data) from the optical system 412 and or the optical system controller 413. In some embodiments, the algorithm processor 416 calculates one or more physiological parameters and can analyze the spectral information. Thus, for example and without limitation, a model can be used that determines, based on the spectral information, physiological parameters of fluid from the fluid source 402. The algorithm processor 416, a controller that may be part of the display system 414, and any embedded controllers within the system 400 can be connected to one another with a communications bus.

Some embodiments of the systems described herein (e.g., the system 400), as well as some embodiments of each method described herein, can include a physical computer program accessible to and/or executable by a processing system, e.g., a one or more processors, controllers, and memories that are part of an embedded system. Indeed, the controllers may comprise one or more physical computing devices and/or may use software. Thus, as will be appreciated by those skilled in the art, various embodiments may be embodied as a method, an apparatus such as a special purpose apparatus, an apparatus such as a data processing system, or a carrier medium, e.g., a non-transitory computer program product. The carrier medium carries one or more computer readable code segments for controlling a processing system to implement a method. The carrier medium can include non-transitory storage media or carrier signals. Accordingly, various embodiments may take the form of a method, an entirely hardware embodiment, an entirely software embodiment or an embodiment combining software and hardware aspects. Furthermore, any one or more of the disclosed methods (including but not limited to the disclosed methods of measurement analysis, interferent determination, and/or calibration constant generation) may be stored as one or more computer readable code segments or data compilations on a carrier medium. Any suitable computer readable carrier medium may be used including a magnetic storage device such as a diskette or a hard disk; a memory cartridge, module, card or chip (either alone or installed within a larger device); or an optical storage device such as a CD or DVD.

### Fluid Handling System

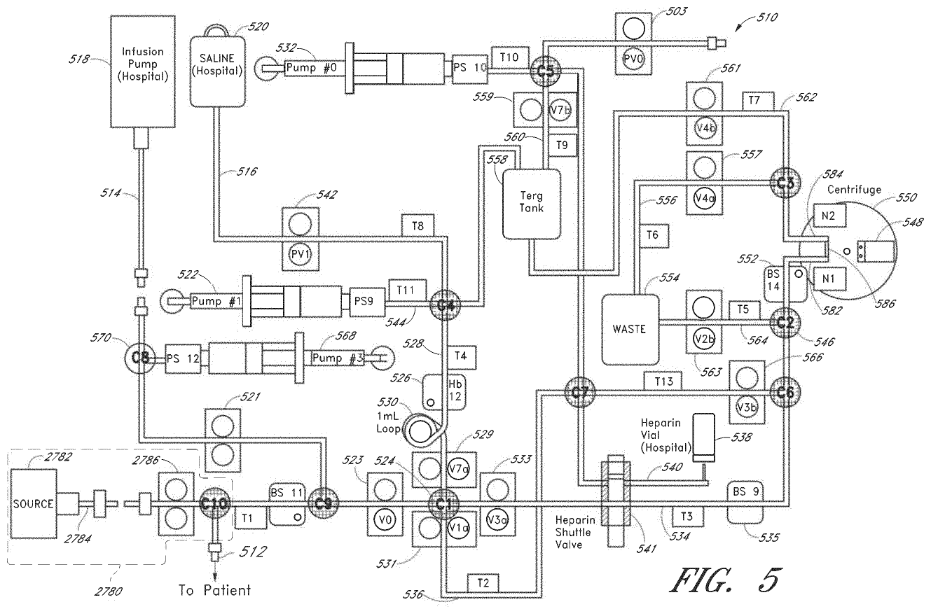

The generalized fluid-handling system 404 can have various configurations. In this context, FIG. 5 schematically illustrates the layout of an example embodiment of a fluid system 510. In this schematic representation, various components are depicted that may be part of a non-disposable subsystem 406, a first disposable subsystem 408, a second disposable subsystem 410, and/or an optical system 412. The fluid system 510 is described practically to show an example cycle as fluid is drawn and analyzed.

In addition to the reference numerals used below, the various portions of the illustrated fluid system 510 are labeled for convenience with letters to suggest their roles as follows: T# indicates a section of tubing. C# indicates a connector that joins multiple tubing sections. V# indicates a valve. BS# indicates a bubble sensor or ultrasonic air detector. N# indicates a needle (e.g., a needle that injects sample into a sample holder). PS# indicates a pressure sensor (e.g., a reusable pressure sensor). Pump# indicates a fluid pump (e.g., a syringe pump with a disposable body and reusable drive). “Hb 12” indicates a sensor for hemoglobin (e.g., a dilution sensor that can detect hemoglobin optically).

The term “valve” as used herein is a broad term and is used, in accordance with its ordinary meaning, to refer to any flow regulating device. For example, the term “valve” can include, without limitation, any device or system that can controllably allow, prevent, or inhibit the flow of fluid through a fluid passageway. The term “valve” can include some or all of the following, alone or in combination: pinch valves, rotary valves, stop cocks, pressure valves, shuttle valves, mechanical valves, electrical valves, electro-mechanical flow regulators, etc. In some embodiments, a valve can regulate flow using gravitational methods or by applying electrical voltages or by both.

The term “pump” as used herein is a broad term and is used, in accordance with its ordinary meaning, to refer to any device that can urge fluid flow. For example, the term “pump” can include any combination of the following: syringe pumps, peristaltic pumps, vacuum pumps, electrical pumps, mechanical pumps, hydraulic pumps, etc. Pumps and/or pump components that are suitable for use with some embodiments can be obtained, for example, from or through Hospira.

The function of the valves, pumps, actuators, drivers, motors (e.g., the centrifuge motor), etc. described below is controlled by one or more controllers (e.g., the fluid system controller 405, the optical system controller 413, etc.) The controllers can include software, computer memory, electrical and mechanical connections to the controlled components, etc.

At the start of a measurement cycle, most lines, including a patient tube 512 (T1), an Arrival sensor tube 528 (T4), an anticoagulant valve tube 534 (T3), and a sample cell 548 can be filled with saline that can be introduced into the system through the infusion tube 514 and the saline tube 516, and which can come from an infusion pump 518 and/or a saline bag 520. The infusion pump 518 and the saline bag 520 can be provided separately from the system 510. For example, a hospital can use existing saline bags and infusion pumps to interface with the described system. The infusion valve 521 can be open to allow saline to flow into the tube 512 (T1).

Before drawing a sample, the saline in part of the system 510 can be replaced with air. Thus, for example, the following valves can be closed: air valve 503 (PV0), the detergent tank valve 559 (V7b), 566 (V3b), 523 (V0), 529 (V7a), and 563 (V2b). At the same time, the following valves can be open: valves 531 (Via), 533 (V3a) and 577 (V4a). Simultaneously, a second pump 532 (pump #0) pumps air through the system 510 (including tube 534 (T3), sample cell 548, and tube 556 (T6)), pushing saline through tube 534 (T3) and sample cell 548 into a waste bladder 554.

Next, a sample can be drawn. With the valves 542 (PV1), 559 (V7b), and 561 (V4b) closed, a first pump 522 (pump #1) is actuated to draw sample fluid to be analyzed (e.g. blood) from a fluid source (e.g., a laboratory sample container, a living patient, etc.) up into the patient tube 512 (T1), through the tube past the two flanking portions of the open pinch-valve 523 (V0), through the first connector 524 (C1), into the looped tube 530, past the arrival sensor 526 (Hb12), and into the arrival sensor tube 528 (T4). The arrival sensor 526 may be used to detect the presence of blood in the tube 528 (T4). For example in some embodiments, the arrival sensor 526 may comprise a hemoglobin sensor. In some other embodiments, the arrival sensor 526 may comprise a color sensor that detects the color of fluid flowing through the tube 528 (T4). During this process, the valve 529 (V7a) and 523 (V0) are open to fluid flow, and the valves 531 (Via), 533 (V3a), 542 (PV1), 559 (V7b), and 561 (V4b) can be closed and therefore block (or substantially block) fluid flow by pinching the tube.

Before drawing the sample, the tubes 512 (T1) and 528 (T4) are filled with saline and the hemoglobin (Hb) level is zero. The tubes that are filled with saline are in fluid communication with the sample source (e.g., the fluid source 402). The sample source can be the vessels of a living human or a pool of liquid in a laboratory sample container, for example. When the saline is drawn toward the first pump 522, fluid to be analyzed is also drawn into the system because of the suction forces in the closed fluid system. Thus, the first pump 522 draws a relatively continuous column of fluid that first comprises generally nondiluted saline, then a mixture of saline and sample fluid (e.g., blood), and then eventually nondiluted sample fluid. In the example illustrated here, the sample fluid is blood.

The arrival sensor 526 (Hb12) can detect and/or verify the presence of blood in the tubes. For example, in some embodiments, the arrival sensor 526 can determine the color of the fluid in the tubes. In some embodiments, the arrival sensor 526 (Hb12) can detect the level of Hemoglobin in the sample fluid. As blood starts to arrive at the arrival sensor 526 (Hb12), the sensed hemoglobin level rises. A hemoglobin level can be selected, and the system can be pre-set to determine when that level is reached. A controller such as the fluid system controller 405 of FIG. 4 can be used to set and react to the pre-set value, for example. In some embodiments, when the sensed hemoglobin level reaches the pre-set value, substantially undiluted sample is present at the first connector 524 (C1). The preset value can depend, in part, on the length and diameter of any tubes and/or passages traversed by the sample. In some embodiments, the pre-set value can be reached after approximately 2 mL of fluid (e.g., blood) has been drawn from a fluid source. A nondiluted sample can be, for example, a blood sample that is not diluted with saline solution, but instead has the characteristics of the rest of the blood flowing through a patient's body. A loop of tubing 530 (e.g., a 1-mL loop) can be advantageously positioned as illustrated to help insure that undiluted fluid (e.g., undiluted blood) is present at the first connector 524 (C1) when the arrival sensor 526 registers that the preset Hb threshold is crossed. The loop of tubing 530 provides additional length to the Arrival sensor tube 528 (T4) to make it less likely that the portion of the fluid column in the tubing at the first connector 524 (C1) has advanced all the way past the mixture of saline and sample fluid, and the nondiluted blood portion of that fluid has reached the first connector 524 (C1).

In some embodiments, when nondiluted blood is present at the first connector 524 (C1), a sample is mixed with an anticoagulant and is directed toward the sample cell 548. An amount of anticoagulant (e.g., heparin) can be introduced into the tube 534 (T3), and then the undiluted blood is mixed with the anticoagulant. A heparin vial 538 (e.g., an insertable vial provided independently by the user of the system 510) can be connected to a tube 540. An anticoagulant valve 541 (which can be a shuttle valve, for example) can be configured to connect to both the tube 540 and the anticoagulant valve tube 534 (T3). The valve can open the tube 540 to a suction force (e.g., created by the pump 532), allowing heparin to be drawn from the vial 538 into the valve 541. Then, the anticoagulant valve 541 can slide the heparin over into fluid communication with the anticoagulant valve tube 534 (T3). The anticoagulant valve 541 can then return to its previous position. Thus, heparin can be shuttled from the tube 540 into the anticoagulant valve tube 534 (T3) to provide a controlled amount of heparin into the tube 534 (T3).

With the valves 542 (PV1), 559 (V7b), 561 (V4b), 523 (V0), 531 (Via), 566 (V3b), and 563 (V2b) closed, and the valves 529 (V7a) and 533 (V3a) open, first pump 522 (pump #1) pushes the sample from tube 528 (T4) into tube 534 (T3), where the sample mixes with the heparin injected by the anticoagulant valve 541 as it flows through the system 510. As the sample proceeds through the tube 534 (T3), the air that was previously introduced into the tube 534 (T3) is displaced. The sample continues to flow until a bubble sensor 535 (BS9) indicates a change from air to a liquid, and thus the arrival of a sample at the bubble sensor. In some embodiments, the volume of tube 534 (T3) from connector 524 (C1) to bubble sensor 535 (BS9) is a known and/or engineered amount, and may be approximately 500 μL, 200 μL or 100 μL, for example. In some embodiments, the volume of tube 534 (T3) from connector 524 (C1) to bubble sensor 535 (BS9) may be approximately less than 10 ml.

When bubble sensor 535 (BS9) indicates the presence of a sample, the remainder of the sampled blood can be returned to its source (e.g., the patient veins or arteries). The first pump 522 (pump #1) pushes the blood out of the Arrival sensor tube 528 (T4) and back to the patient by opening the valve 523 (V0), closing the valves 531 (Via) and 533 (V3a), and keeping the valve 529 (V7a) open. The Arrival sensor tube 528 (T4) is preferably flushed with approximately 2 mL of saline. This can be accomplished by closing the valve 529 (V7a), opening the valve 542 (PV1), drawing saline from the saline source 520 into the tube 544, closing the valve 542 (PV1), opening the valve 529 (V7a), and forcing the saline down the Arrival sensor tube 528 (T4) with the pump 522. In some embodiments, less than two minutes elapse between the time that blood is drawn from the patient and the time that the blood is returned to the patient.

Following return of the unused patient blood sample, the sample is pushed up the anticoagulant valve tube 534 (T3), through the second connector 546 (C2), and into the sample cell 548, which can be located on the centrifuge rotor 550. This fluid movement is facilitated by the coordinated action (either pushing or drawing fluid) of the pump 522 (pump #1), the pump 532 (pump #0), and the various illustrated valves. In particular, valve 531 (Via) can be opened, and valves 503 (PV0) and 559 (V7b) can be closed. Pump movement and valve position corresponding to each stage of fluid movement can be coordinated by one ore multiple controllers, such as the fluid system controller 405 of FIG. 4.

After the unused sample is returned to the patient, the sample can be divided into separate slugs before being delivered into the sample cell 548. Thus, for example, valve 533 (V3a) is opened, valves 566 (V3b), 523 (V0) and 529 (V7a) are closed, and the pump 532 (pump #0) uses air to push the sample toward sample cell 548. In some embodiments, the sample (for example, 200 μL or 100 μL) is divided into multiple (e.g., more than two, five, or four) “slugs” of sample, each separated by a small amount of air. As used herein, the term “slug” refers to a continuous column of fluid that can be relatively short. Slugs can be separated from one another by small amounts of air (or bubbles) that can be present at intervals in the tube. In some embodiments, the slugs are formed by injecting or drawing air into fluid in the first connector 546 (C2).

In some embodiments, when the leading edge of the sample reaches blood sensor 552 (BS14), a small amount of air (the first “bubble”) is injected at a connector C6. This bubble helps define the first “slug” of liquid, which extends from the bubble sensor to the first bubble. In some embodiments, the valves 533 (V3a) and 566 (V3b) are alternately opened and closed to form a bubble at connector C6, and the sample is pushed toward the sample cell 548. Thus, for example, with pump 532 actuated, valve 566 V(3b) is briefly opened and valve 533 (V3a) is briefly closed to inject a first air bubble into the sample.

In some embodiments, the volume of the tube 534 (T3) from the connector 546 (C2) to the bubble sensor 552 (BS14) is less than the volume of tube 534 (T3) from the connector 524 (C1) to the bubble sensor 535 (BS9). Thus, for example and without limitation, the volume of the tube 534 (T3) from the connector 524 (C1) to the bubble sensor 535 (BS9) can be in the range of approximately 80 μL to approximately 120 μL, (e.g., 100 μL) and the volume of the tube 534 (T3) from the connector 546 (C2) to the bubble sensor 552 (BS14) can be in the range of approximately 5 μL to approximately 25 μL (e.g., 15 μL). In some embodiments, multiple blood slugs are created. For example, more than two blood slugs can be created, each having a different volume. In some embodiments, five blood slugs are created, each having approximately the same volume of approximately 20 μL each. In some embodiments, three blood slugs are created, the first two having a volume of 10 μL and the last having a volume of 20 μL. In some embodiments, four blood slugs are created; the first three blood slugs can have a volume of approximately 154, and the fourth can have a volume of approximately 35 μL.

A second slug can be prepared by opening the valve 533 (V3a), closing the valve 566 (V3b), with pump 532 (pump #0) operating to push the first slug through a first sample cell holder interface tube 582 (N1), through the sample cell 548, through a second sample cell holder interface tube 584 (N2), and toward the waste bladder 554. When the first bubble reaches the bubble sensor 552 (BS 14), the open/closed configurations of valves 533 (V3a) and 566 (V3b) are reversed, and a second bubble is injected into the sample, as before. A third slug can be prepared in the same manner as the second (pushing the second bubble to bubble sensor 552 (BS 14) and injecting a third bubble). After the injection of the third air bubble, the sample can be pushed through system 510 until the end of the sample is detected by bubble sensor 552 (BS 14). The system can be designed such that when the end of the sample reaches this point, the last portion of the sample (a fourth slug) is within the sample cell 548, and the pump 532 can stop forcing the fluid column through the anticoagulant valve tube 534 (T3) so that the fourth slug remains within the sample cell 548. Thus, the first three blood slugs can serve to flush any residual saline out the sample cell 548. The three leading slugs can be deposited in the waste bladder 554 by passing through the tube 556 (T6) and past the tube-flanking portions of the open pinch valve 557 (V4a).

In some embodiments, the fourth blood slug is centrifuged for a given length of time (e.g., more than 1 minute, five minutes, or 2 minutes, to take three advantageous examples) at a relatively fast speed (e.g., 7200 RPM, 5000 RPM, or 4500 RPM, to take three examples). Thus, for example, the sample cell holder interface tubes 582 (N1) and 584 (N2) disconnect the sample cell 548 from the tubes 534 (T3) and 562 (T7), permitting the centrifuge rotor 550 and the sample cell 548 to spin together. Spinning separates a sample (e.g., blood) into its components, isolates the plasma, and positions the plasma in the sample cell 548 for measurement. The centrifuge 550 can be stopped with the sample cell 548 in a beam of radiation (not shown) for analysis. The radiation, a detector, and logic can be used to analyze a portion of the sample (e.g., the plasma) spectroscopically (e.g., for glucose, lactate, or other analyte concentration). In some embodiments, some or all of the separated components (e.g., the isolated plasma) may be transported to a different analysis chamber. For example, another analysis chamber can have one or more electrodes in electrical communication with the chamber's contents, and the separated components may be analyzed electrically. At any suitable point, one or more of the separated components can be transported to the waste bladder 554 when no longer needed. In some chemical analysis systems and apparatus, the separated components are analyzed electrically. Analysis devices may be connected serially, for example, so that the analyzed substance from an optical analysis system (e.g., an “OptiScanner®” fluid analyzer) can be transferred to an independent analysis device (e.g., a chemical analysis device) for subsequent analysis. In certain embodiments, the analysis devices are integrated into a single system. Many variations are possible.

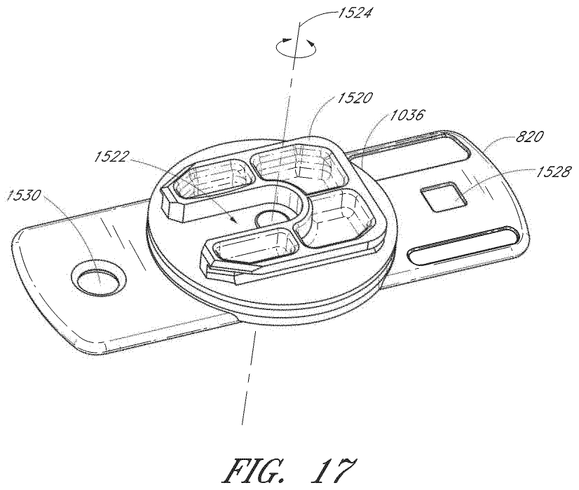

In some embodiments, portions of the system 510 that contain blood after the sample cell 548 has been provided with a sample are cleaned to prevent blood from clotting. Accordingly, the centrifuge rotor 550 can include two passageways for fluid that may be connected to the sample cell holder interface tubes 582 (N1) and 584 (N2). One passageway is sample cell 548, and a second passageway is a shunt 586. An embodiment of the shunt 586 is illustrated in more detail in FIG. 16 (see reference numeral 1586).

The shunt 586 can allow cleaner (e.g., a detergent such as tergazyme A) to flow through and clean the sample cell holder interface tubes without flowing through the sample cell 548. After the sample cell 548 is provided with a sample, the interface tubes 582 (N1) and 584 (N2) are disconnected from the sample cell 548, the centrifuge rotor 550 is rotated to align the shunt 586 with the interface tubes 582 (N1) and 584 (N2), and the interface tubes are connected with the shunt. With the shunt in place, the detergent tank 559 is pressurized by the second pump 532 (pump #0) with valves 561 (V4b) and 563 (V2b) open and valves 557 (V4a) and 533 (V3a) closed to flush the cleaning solution back through the interface tubes 582 (N1) and 584 (N2) and into the waste bladder 554. Subsequently, saline can be drawn from the saline bag 520 for a saline flush. This flush pushes saline through the Arrival sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (Via), 566 (V3b), 563 (V2b), and 561 (V4b).

Following analysis, the second pump 532 (pump #0) flushes the sample cell 548 and sends the flushed contents to the waste bladder 554. This flush can be done with a cleaning solution from the detergent tank 558. In some embodiments, the detergent tank valve 559 (V7b) is open, providing fluid communication between the second pump 532 and the detergent tank 558. The second pump 532 forces cleaning solution from the detergent tank 558 between the tube-flanking portions of the open pinch valve 561 and through the tube 562 (T7). The cleaning flush can pass through the sample cell 548, through the second connector 546, through the tube 564 (T5) and the open valve 563 (V2b), and into the waste bladder 554.

Subsequently, the first pump 522 (pump #1) can flush the cleaning solution out of the sample cell 548 using saline in drawn from the saline bag 520. This flush pushes saline through the Arrival sensor tube 528 (T4), the anticoagulant valve tube 534 (T3), the sample cell 548, and the waste tube 556 (T6). Thus, in some embodiments, the following valves are open for this flush: 529 (V7a), 533 (V3a), 557 (V4a), and the following valves are closed: 542 (PV1), 523 (V0), 531 (Via), 566 (V3b), 563 (V2b), and 561 (V4b).

When the fluid source is a living entity such as a patient, a low flow of saline (e.g., 1-5 mL/hr) is preferably moved through the patient tube 512 (T1) and into the patient to keep the patient's vessel open (e.g., to establish a keep vessel open, or “KVO” flow). This KVO flow can be temporarily interrupted when fluid is drawn into the fluid system 510. The source of this KVO flow can be the infusion pump 518, the third pump 568 (pump #3), or the first pump 522 (pump #1). In some embodiments, the infusion pump 518 can run continuously throughout the measurement cycle described above. This continuous flow can advantageously avoid any alarms that may be triggered if the infusion pump 518 senses that the flow has stopped or changed in some other way. In some embodiments, when the infusion valve 521 closes to allow pump 522 (pump #1) to withdraw fluid from a fluid source (e.g., a patient), the third pump 568 (pump #3) can withdraw fluid through the connector 570, thus allowing the infusion pump 518 to continue pumping normally as if the fluid path was not blocked by the infusion valve 521. If the measurement cycle is about two minutes long, this withdrawal by the third pump 568 can continue for approximately two minutes. Once the infusion valve 521 is open again, the third pump 568 (pump #3) can reverse and insert the saline back into the system at a low flow rate. Preferably, the time between measurement cycles is longer than the measurement cycle itself (for example, the time interval can be longer than ten minutes, shorter than ten minutes, shorter than five minutes, longer than two minutes, longer than one minute, etc.). Accordingly, the third pump 568 can insert fluid back into the system at a lower rate than it withdrew that fluid. This can help prevent an alarm by the infusion pump.

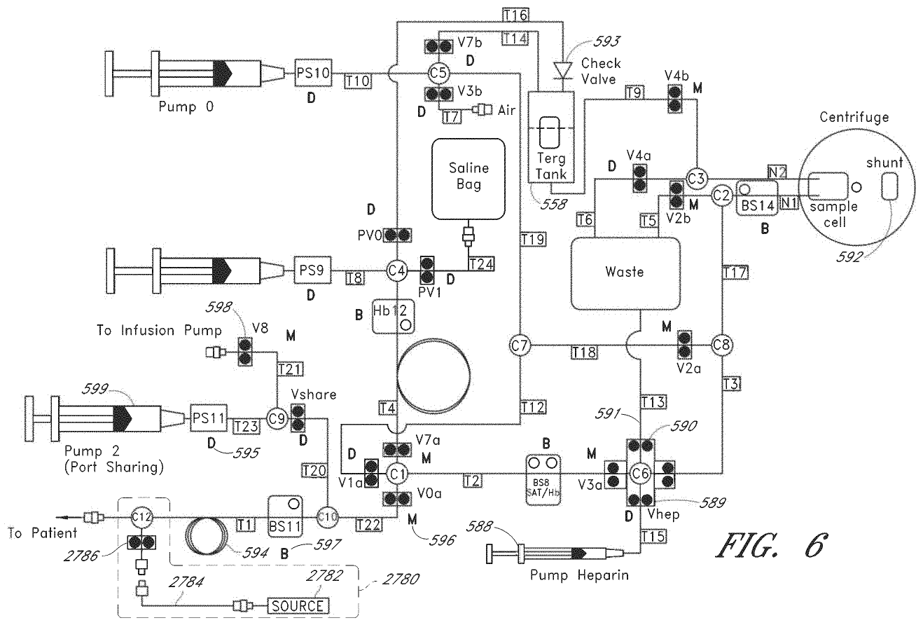

FIG. 6 schematically illustrates another embodiment of a fluid system that can be part of a system for withdrawing and analyzing fluid samples. In this embodiment, the anticoagulant valve 541 has been replaced with a syringe-style pump 588 (Pump Heparin) and a series of pinch valves around a junction between tubes. For example, a heparin pinch valve 589 (Vhep) can be closed to prevent flow from or to the pump 588, and a heparin waste pinch valve 590 can be closed to prevent flow from or to the waste container from this junction through the heparin waste tube 591. This embodiment also illustrates the shunt 592 schematically. Other differences from FIG. 5 include the check valve 593 located near the detergent tank 558 and the patient loop 594. The reference letters D, for example, the one indicated at 595, refer to components that are advantageously located on the door. The reference letters M, for example, the one indicated at 596, refer to components that are advantageously located on the monitor. The reference letters B, for example, the one indicated at 597, refer to components that can be advantageously located on both the door and the monitor.

In some embodiments, the system 400 (see FIG. 4), the apparatus 100 (see FIG. 1), or even the monitoring device 102 (see FIG. 1) itself can also actively function not only to monitor analyte levels (e.g., glucose), but also to change and/or control analyte levels. Thus, the monitoring device 102 can be both a monitoring and an infusing device. In some embodiments, the fluid handling system 510 can include an optional analyte control subsystem 2780 that will be further described below (see discussion of analyte control).

In certain embodiments, analyte levels in a patient can be adjusted directly (e.g., by infusing or extracting glucose) or indirectly (e.g., by infusing or extracting insulin). FIG. 6 illustrates one way of providing this function. The infusion pinch valve 598 (V8) can allow the port sharing pump 599 (compare to the third pump 568 (pump #3) in FIG. 5) to serve two roles. In the first role, it can serve as a “port sharing” pump. The port sharing function is described with respect to the third pump 568 (pump #3) of FIG. 5, where the third pump 568 (pump #3) can withdraw fluid through the connector 570, thus allowing the infusion pump 518 to continue pumping normally as if the fluid path was not blocked by the infusion valve 521. In the second role, the port sharing pump 599 can serve as an infusion pump. The infusion pump role allows the port sharing pump 599 to draw a substance (e.g., glucose, saline, etc.) from another source when the infusion pinch valve 598 is open, and then to infuse that substance into the system or the patient when the infusion pinch valve 598 is closed. This can occur, for example, in order to change the level of a substance in a patient in response to a reading by the monitor that the substance is too low. In some embodiments, one or more of the pumps may comprise a reversible infusion pump configured to interrupt the flow of the infusion fluid and draw a sample of blood for analysis.

### Mechanical/Fluid System Interface

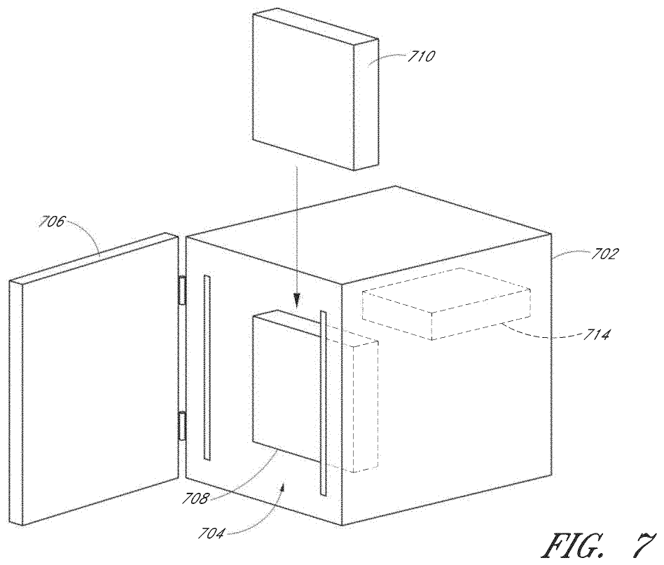

FIG. 7 is an oblique schematic depiction of a modular monitoring device 700, which can correspond to the monitoring device 102. The modular monitoring device 700 includes a body portion 702 having a receptacle 704, which can be accessed by moving a movable portion 706. The receptacle 704 can include connectors (e.g., rails, slots, protrusions, resting surfaces, etc.) with which a removable portion 710 can interface. In some embodiments, portions of a fluidic system that directly contact fluid are incorporated into one or more removable portions (e.g., one or more disposable cassettes, sample holders, tubing cards, etc.). For example, a removable portion 710 can house at least a portion of the fluid system 510 described previously, including portions that contact sample fluids, saline, detergent solution, and/or anticoagulant.

In some embodiments, a non-disposable fluid-handling subsystem 708 is disposed within the body portion 702 of the monitoring device 700. The first removable portion 710 can include one or more openings that allow portions of the non-disposable fluid-handling subsystem 708 to interface with the removable portion 710. For example, the non-disposable fluid-handling subsystem 708 can include one or more pinch valves that are designed to extend through such openings to engage one or more sections of tubing. When the first removable portion 710 is present in a corresponding first receptacle 704, actuation of the pinch valves can selectively close sections of tubing within the removable portion. The non-disposable fluid-handling subsystem 708 can also include one or more sensors that interface with connectors, tubing sections, or pumps located within the first removable portion 710. The non-disposable fluid-handling subsystem 708 can also include one or more actuators (e.g., motors) that can actuate moveable portions (e.g., the plunger of a syringe) that may be located in the removable portion F10. A portion of the non-disposable fluid-handling subsystem 708 can be located on or in the moveable portion F06 (which can be a door having a slide or a hinge, a detachable face portion, etc.).

In the embodiment shown in FIG. 7, the monitoring device 700 includes an optical system 714 disposed within the body portion 702. The optical system 714 can include a light source and a detector that are adapted to perform measurements on fluids within a sample holder (not shown). The light source may comprise a fixed wavelength light source and/or a tunable light source. The light source may comprise one or more sources including, for example, broadband sources, LEDs, and lasers. In some embodiments, the sample holder comprises a removable portion, which can be associated with or disassociated from the removable portion F10. The sample holder can include an optical window through which the optical system 714 can emit radiation for measuring properties of a fluid in the sample holder. The optical system 714 can include other components such as, for example, a power supply, a centrifuge motor, a filter wheel, and/or a beam splitter.

In some embodiments, the removable portion 710 and the sample holder are adapted to be in fluid communication with each other. For example, the removable portion 710 can include a retractable injector that injects fluids into a sample holder. In some embodiments, the sample holder can comprise or be disposed in a second removable portion (not shown). In some embodiments, the injector can be retracted to allow the centrifuge to rotate the sample holder freely.

The body portion 702 of the monitoring device 700 can also include one or more connectors for an external battery (not shown). The external battery can serve as a backup emergency power source in the event that a primary emergency power source such as, for example, an internal battery (not shown) is exhausted.

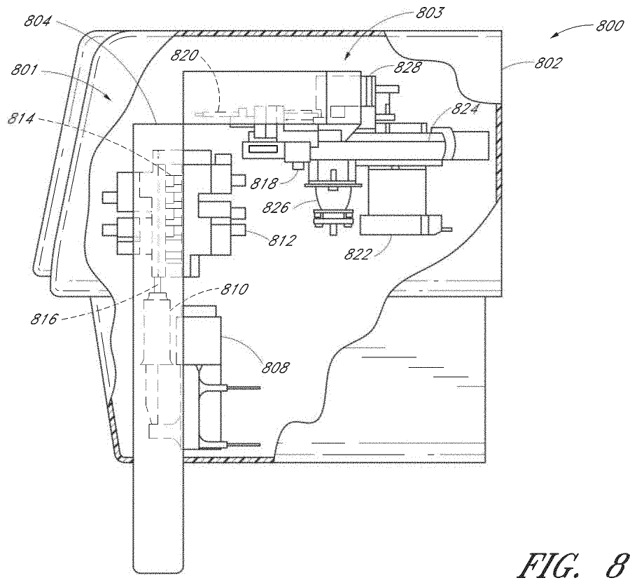

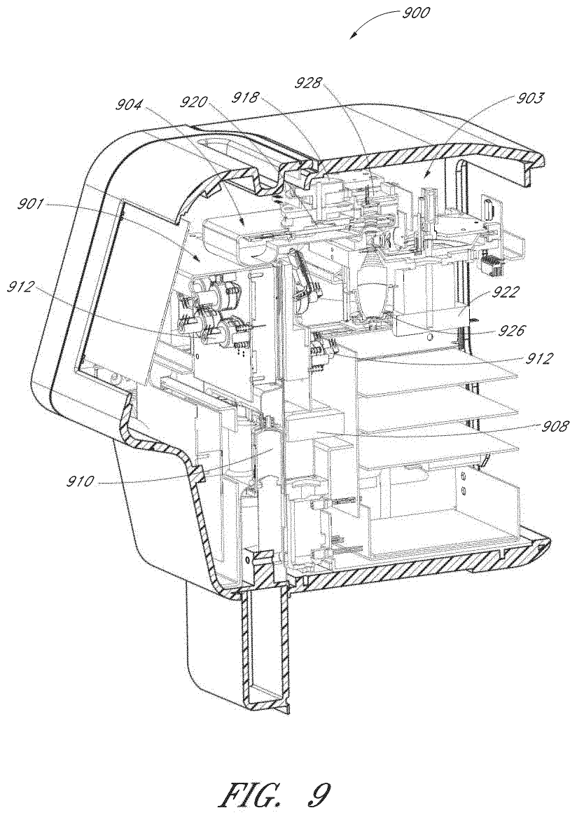

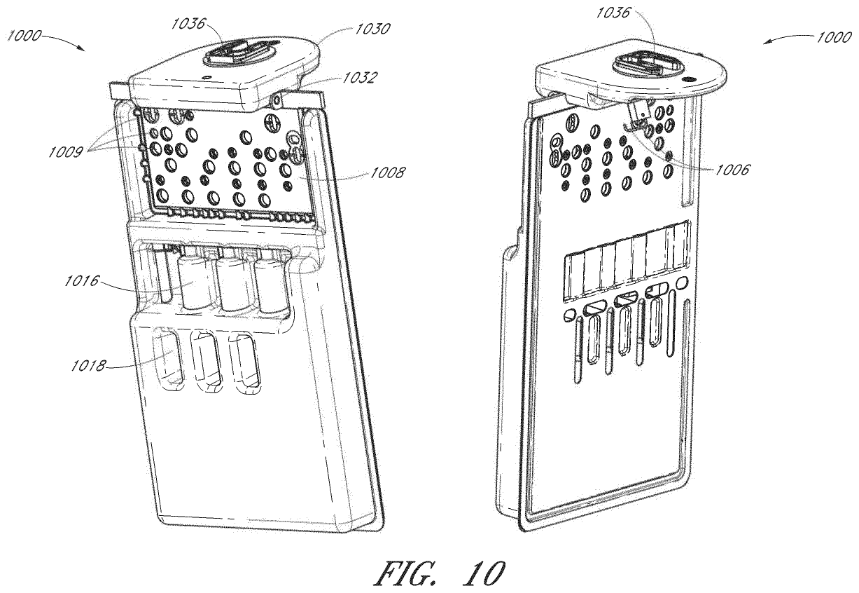

FIG. 7 shows an embodiment of a system having subcomponents illustrated schematically. By way of a more detailed (but nevertheless non-limiting) example, FIG. 8 and FIG. 9 show more details of the shape and physical configuration of a sample embodiment.

FIG. 8 shows a cut-away side view of a monitoring device 800 (which can correspond, for example, to the device 102 shown in FIG. 1). The device 800 includes a casing 802. The monitoring device 800 can have a fluid system. For example, the fluid system can have subsystems, and a portion or portions thereof can be disposable, as schematically depicted in FIG. 4. As depicted in FIG. 8, the fluid system is generally located at the left-hand portion of the casing 802, as indicated by the reference 801. The monitoring device 800 can also have an optical system. In the illustrated embodiment, the optical system is generally located in the upper portion of the casing 802, as indicated by the reference 803. Advantageously, however, the fluid system 801 and the optical system 803 can both be integrated together such that fluid flows generally through a portion of the optical system 803, and such that radiation flows generally through a portion of the fluid system 801.

Depicted in FIG. 8 are examples of ways in which components of the device 800 mounted within the casing 802 can interface with components of the device 800 that comprise disposable portions. Not all components of the device 800 are shown in FIG. 8. A disposable portion 804 having a variety of components is shown in the casing 802. In some embodiments, one or more actuators 808 housed within the casing 802, operate syringe bodies 810 located within a disposable portion 804. The syringe bodies 810 are connected to sections of tubing 816 that move fluid among various components of the system. The movement of fluid is at least partially controlled by the action of one or more pinch valves 812 positioned within the casing 802. The pinch valves 812 have arms 814 that extend within the disposable portion 804. Movement of the arms 814 can constrict a section of tubing 816.

In some embodiments, a sample cell holder 820 can engage a centrifuge motor 818 mounted within the casing 802 of the device 800. A filter wheel motor 822 disposed within the housing 802 rotates a filter wheel 824, and in some embodiments, aligns one or more filters with an optical path. An optical path can originate at a source 826 within the housing 802 that can be configured to emit a beam of radiation (e.g., infrared radiation, visible radiation, ultraviolet radiation, etc.) through the filter and the sample cell holder 820 and to a detector 828. A detector 828 can measure the optical density of the light when it reaches the detector.

FIG. 9 shows a cut-away perspective view of an alternative embodiment of a monitoring device 900. Many features similar to those illustrated in FIG. 8 are depicted in this illustration of an alternative embodiment. A fluid system 901 can be partially seen. The disposable portion 904 is shown in an operative position within the device. One of the actuators 808 can be seen next to a syringe body 910 that is located within the disposable portion 904. Some pinch valves 912 are shown next to a fluid-handling portion of the disposable portion 904. In this figure, an optical system 903 can also be partially seen. The sample holder 920 is located underneath the centrifuge motor 918. The filter wheel motor 922 is positioned near the radiation source 926, and the detector 928 is also illustrated.

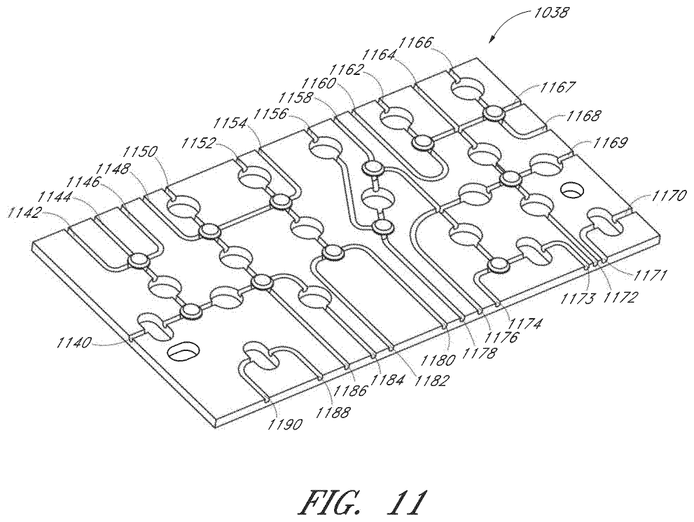

FIG. 10 illustrates two views of a cartridge 1000 that can interface with a fluid system such as the fluid system 510 of FIG. 5. The cartridge 1000 can be configured for insertion into a receptacle of the device 800 of FIG. 8 and/or the device 900 shown in FIG. 9. In some embodiments, the cartridge 1000 can comprise a portion that is disposable and a portion that is reusable. In some embodiments, the cartridge 1000 can be disposable. The cartridge 1000 can fill the role of the removable portion 710 of FIG. 7, for example. In some embodiments, the cartridge 1000 can be used for a system having only one disposable subsystem, making it a simple matter for a health care provider to replace and/or track usage time of the disposable portion. In some embodiments, the cartridge 1000 includes one or more features that facilitate insertion of the cartridge 1000 into a corresponding receptacle. For example, the cartridge 1000 can be shaped so as to promote insertion of the cartridge 1000 in the correct orientation. The cartridge 1000 can also include labeling or coloring affixed to or integrated with the cartridge's exterior casing that help a handler insert the cartridge 1000 into a receptacle properly.

The cartridge 1000 can include one or more ports for connecting to material sources or receptacles. Such ports can be provided to connect to, for example, a saline source, an infusion pump, a sample source, and/or a source of gas (e.g., air, nitrogen, etc.). The ports can be connected to sections of tubing within the cartridge 1000. In some embodiments, the sections of tubing are opaque or covered so that fluids within the tubing cannot be seen, and in some embodiments, sections of tubing are transparent to allow interior contents (e.g., fluid) to be seen from outside.

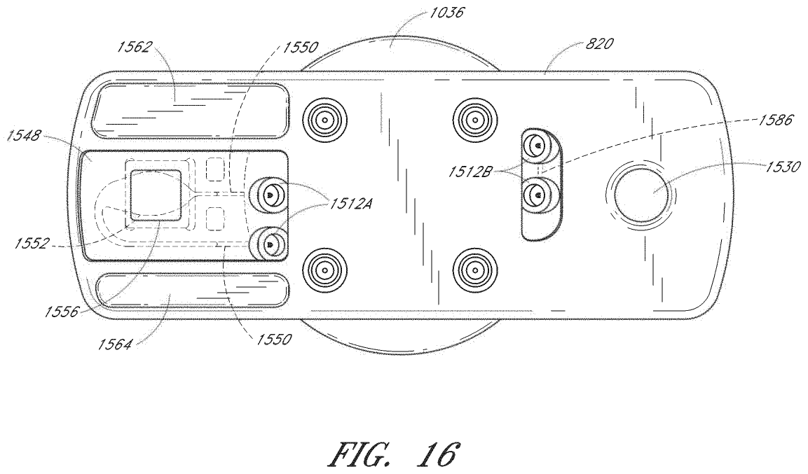

The cartridge 1000 shown in FIG. 10 can include a sample injector 1006. The sample injector 1006 can be configured to inject at least a portion of a sample into a sample holder (see, e.g., the sample cell 548), which can also be incorporated into the cartridge 1000. The sample injector 1006 can include, for example, the sample cell holder interface tubes 582 (N1) and 584 (N2) of FIG. 5, embodiments of which are also illustrated in FIG. 15.

The housing of the cartridge 1000 can include a tubing portion 1008 containing within it a card having one or more sections of tubing. In some embodiments, the body of the cartridge 1000 includes one or more apertures 1009 through which various components, such as, for example, pinch valves and sensors, can interface with the fluid-handling portion contained in the cartridge 1000. The sections of tubing found in the tubing portion 1008 can be aligned with the apertures 1009 in order to implement at least some of the functionality shown in the fluid system 510 of FIG. 5.

The cartridge 1000 can include a pouch space (not shown) that can comprise one or more components of the fluid system 510. For example, one or more pouches and/or bladders can be disposed in the pouch space (not shown). In some embodiments, a cleaner pouch and/or a waste bladder can be housed in a pouch space. The waste bladder can be placed under the cleaner pouch such that, as detergent is removed from the cleaner pouch, the waste bladder has more room to fill. The components placed in the pouch space (not shown) can also be placed side-by-side or in any other suitable configuration.

The cartridge 1000 can include one or more pumps 1016 that facilitate movement of fluid within the fluid system 510. Each of the pump housings 1016 can contain, for example, a syringe pump having a plunger. The plunger can be configured to interface with an actuator outside the cartridge 1000. For example, a portion of the pump that interfaces with an actuator can be exposed to the exterior of the cartridge 1000 housing by one or more apertures 1018 in the housing.

The cartridge 1000 can have an optical interface portion 1030 that is configured to interface with (or comprise a portion of) an optical system. In the illustrated embodiment, the optical interface portion 1030 can pivot around a pivot structure 1032. The optical interface portion 1030 can house a sample holder (not shown) in a chamber that can allow the sample holder to rotate. The sample holder can be held by a centrifuge interface 1036 that can be configured to engage a centrifuge motor (not shown). When the cartridge 1000 is being inserted into a system, the orientation of the optical interface portion 1030 can be different than when it is functioning within the system.

In some embodiments, the cartridge 1000 is designed for single patient use. The cartridge 1000 may also be disposable and/or designed for replacement after a period of operation. For example, in some embodiments, if the cartridge 1000 is installed in a continuously operating monitoring device that performs four measurements per hour, the waste bladder may become filled or the detergent in the cleaner pouch depleted after about three days. The cartridge 1000 can be replaced before the detergent and waste bladder are exhausted. In some embodiments, a portion of the cartridge 1000 can be disposable while another portion of the cartridge 1000 is disposable, but lasts longer before being discarded. In some embodiments, a portion of the cartridge 1000 may not be disposable at all. For example, a portion thereof may be configured to be cleaned thoroughly and reused for different patients. Various combinations of disposable and less- or non-disposable portions are possible.

The cartridge 1000 can be configured for easy replacement. For example, in some embodiments, the cartridge 1000 is designed to have an installation time of only minutes. For example, the cartridge can be designed to be installed in less than about five minutes, or less than two minutes. During installation, various fluid lines contained in the cartridge 1000 can be primed by automatically filling the fluid lines with saline. The saline can be mixed with detergent powder from the cleaner pouch in order to create a cleaning solution.

The cartridge 1000 can also be designed to have a relatively brief shut down time. For example, the shutdown process can be configured to take less than about fifteen minutes, or less than about ten minutes, or less than about five minutes. The shutdown process can include flushing the patient line; sealing off the insulin pump connection, the saline source connection, and the sample source connection; and taking other steps to decrease the risk that fluids within the used cartridge 1000 will leak after disconnection from the monitoring device.

Some embodiments of the cartridge 1000 can comprise a flat package to facilitate packaging, shipping, sterilizing, etc. Advantageously, however, some embodiments can further comprise a hinge or other pivot structure. Thus, as illustrated, an optical interface portion 1030 can be pivoted around a pivot structure 1032 to generally align with the other portions of the cartridge 1000. The cartridge can be provided to a medical provider sealed in a removable wrapper, for example.

In some embodiments, the cartridge 1000 is designed to fit within standard waste containers found in a hospital, such as a standard biohazard container. For example, the cartridge 1000 can be less than one foot long, less than one foot wide, and less than two inches thick. In some embodiments, the cartridge 1000 is designed to withstand a substantial impact, such as that caused by hitting the ground after a four foot drop, without damage to the housing or internal components. In some embodiments, the cartridge 1000 is designed to withstand significant clamping force applied to its casing. For example, the cartridge 1000 can be built to withstand five pounds per square inch of force without damage. In some embodiments, the cartridge 1000 can be designed to be less sturdy and more biodegradable. In some embodiments, the cartridge 1000 can be formed and configured to withstand more or less than five pounds of force per square inch without damage. In some embodiments, the cartridge 1000 is non pyrogenic and/or latex free.

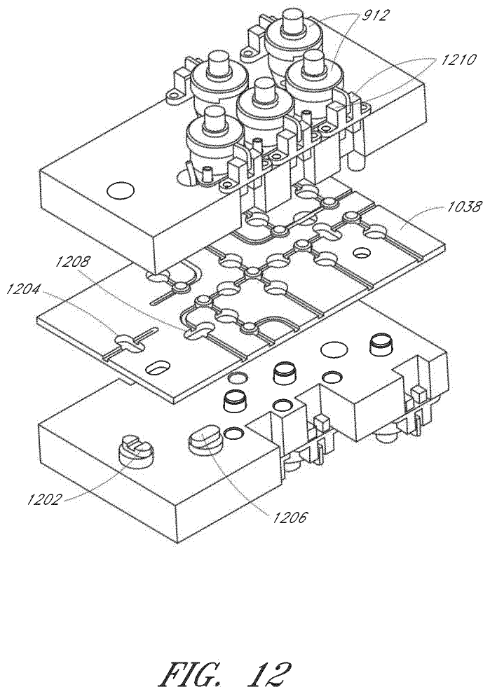

FIG. 11 illustrates an embodiment of a fluid-routing card 1038 that can be part of the removable cartridge of FIG. 10. For example, the fluid-routing card 1038 can be located generally within the tubing portion 1008 of the cartridge 1000. The fluid-routing card 1038 can contain various passages and/or tubes through which fluid can flow as described with respect to FIG. 5 and/or FIG. 6, for example. Thus, the illustrated tube opening openings can be in fluid communication with the following fluidic components, for example:

| Tube Opening | |
|---|---|
| Reference Numeral | Can Be In Fluid Communication With |
| 1142 | third pump 568 (pump #3) |
| 1144 | infusion pump 518 |
| 1146 | Presx |
| 1148 | air pump |
| 1150 | Vent |
| 1152 | detergent (e.g., tergazyme) source or waste tube |
| 1154 | Presx |
| 1156 | detergent (e.g., tergazyme) source or waste tube |
| 1158 | waste receptacle |
| 1160 | first pump 522 (pump #1) (e.g., a saline pump) |
| 1162 | saline source or waste tube |
| 1164 | anticoagulant (e.g., heparin) pump (see FIG. 6) |
| | and/or shuttle valve |
| 1166 | detergent (e.g., tergazyme) source or waste tube |
| 1167 | Presx |
| 1168 | Arrival sensor tube 528 (T4) |
| 1169 | tube 536 (T2) |
| 1170 | Arrival sensor tube 528 (T4) |
| 1171 | Arrival sensor tube 528 (T4) |
| 1172 | anticoagulant (e.g., heparin) pump |
| 1173 | T17 (see FIG. 6) |
| 1174 | Sample cell holder interface tube 582 (N1) |
| 1176 | anticoagulant valve tube 534 (T3) |
| 1178 | Sample cell holder interface tube 584 (N2) |
| 1180 | T17 (see FIG. 6) |
| 1182 | anticoagulant valve tube 534 (T3) |
| 1184 | Arrival sensor tube 528 (T4) |
| 1186 | tube 536 (T2) |
| 1188 | anticoagulant valve tube 534 (T3) |
| 1190 | anticoagulant valve tube 534 (T3) |

The depicted fluid-routing card 1038 can have additional openings that allow operative portions of actuators and/or valves to protrude through the fluid-routing card 1038 and interface with the tubes.

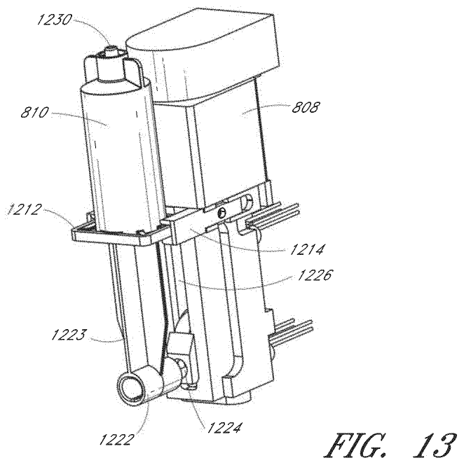

FIG. 12 illustrates how actuators, which can sandwich the fluid-routing card 1038 between them, can interface with the fluid-routing card 1038 of FIG. 11. Pinch valves 812 can have an actuator portion that protrudes away from the fluid-routing card 1038 containing a motor. Each motor can correspond to a pinch platen 1202, which can be inserted into a pinch platen receiving hole 1204. Similarly, sensors, such as a bubble sensor 1206 can be inserted into receiving holes (e.g., the bubble sensor receiving hole 1208). Movement of the pinch valves 812 can be detected by the position sensors 1210.

FIG. 13 illustrates an actuator 808 that is connected to a corresponding syringe body 810. The actuator 808 is an example of one of the actuators 808 that is illustrated in FIG. 8 and in FIG. 9, and the syringe body 810 is an example of one of the syringe bodies 810 that are visible in FIG. 8 and in FIG. 9. A ledge portion 1212 of the syringe body 810 can be engaged (e.g., slid into) a corresponding receiving portion 1214 in the actuator 808. In some embodiments, the receiving portion 1214 can slide outward to engage the stationary ledge portion 1212 after the disposable cartridge 804 is in place. Similarly, a receiving tube 1222 in the syringe plunger 1223 can be slide onto (or can receive) a protruding portion 1224 of the actuator 808. The protruding portion 1224 can slide along a track 1226 under the influence of a motor inside the actuator 808, thus actuating the syringe plunger 1223 and causing fluid to flow into or out of the syringe tip 1230.

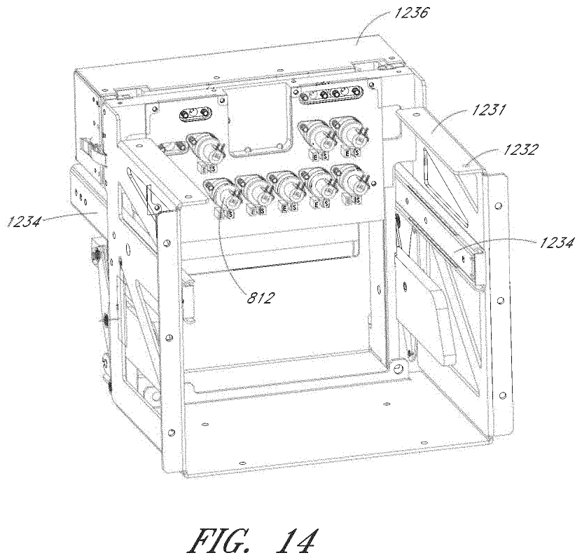

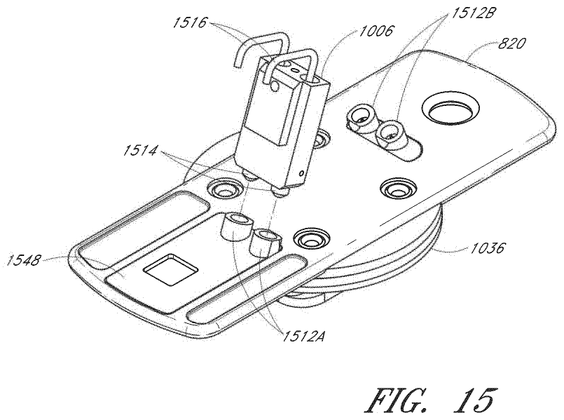

FIG. 14 shows a rear perspective view of internal scaffolding 1231 and the protruding bodies of some pinch valves 812. The internal scaffolding 1231 can be formed from metal and can provide structural rigidity and support for other components. The scaffolding 1231 can have holes 1232 into which screws can be screwed or other connectors can be inserted. In some embodiments, a pair of sliding rails 1234 can allow relative movement between portions of an analyzer. For example, a slidable portion 1236 (which can correspond to the movable portion 706, for example) can be temporarily slid away from the scaffolding 1231 of a main unit in order to allow an insertable portion (e.g., the cartridge 804) to be inserted.

FIG. 15 shows an underneath perspective view of the sample cell holder 820, which is attached to the centrifuge interface 1036. The sample cell holder 820 can have an opposite side (see FIG. 17) that allows it to slide into a receiving portion of the centrifuge interface 1036. The sample cell holder 820 can also have receiving nubs 1512A that provide a pathway into a sample cell 1548 held by the sample cell holder 820. Receiving nubs 1512B can provide access to a shunt 1586 (see FIG. 16) inside the sample cell holder 820. The receiving nubs 1512A and 1512B can receive and or dock with fluid nipples 1514. The fluid nipples 1514 can protrude at an angle from the sample injector 1006, which can in turn protrude from the cartridge 1000 (see FIG. 10). The tubes 1516 shown protruding from the other end of the sample injector 1006 can be in fluid communication with the sample cell holder interface tubes 582 (N1) and 584 (N2) (see FIG. 5 and FIG. 6), as well as 1074 and 1078 (see FIG. 11).

FIG. 16 shows a plan view of the sample cell holder 820 with hidden and/or non-surface portions illustrated using dashed lines. The receiving nubs 1512A communicate with passages 1550 inside the sample cell 1548 (which can correspond, for example to the sample cell 548 of FIG. 5). The passages widen out into a wider portion 1552 that corresponds to a window 1556. The window 1556 and the wider portion 1552 can be configured to house the sample when radiation is emitted along a pathlength that is generally non-parallel to the sample cell 1548. The window 1556 can allow calibration of the instrument with the sample cell 1548 in place, even before a sample has arrived in the wider portion 1552.

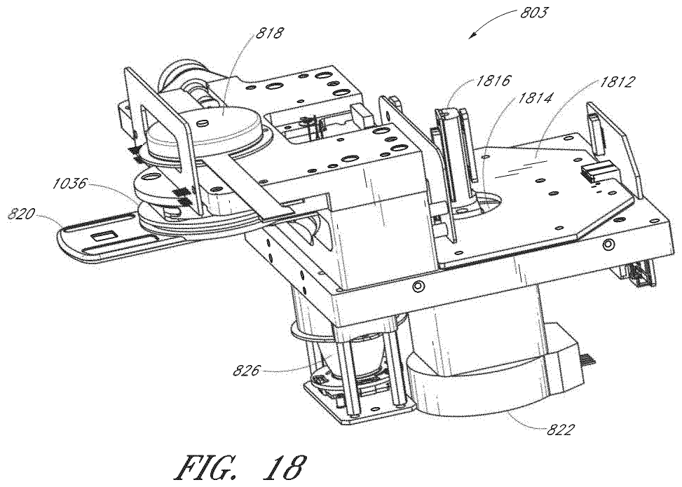

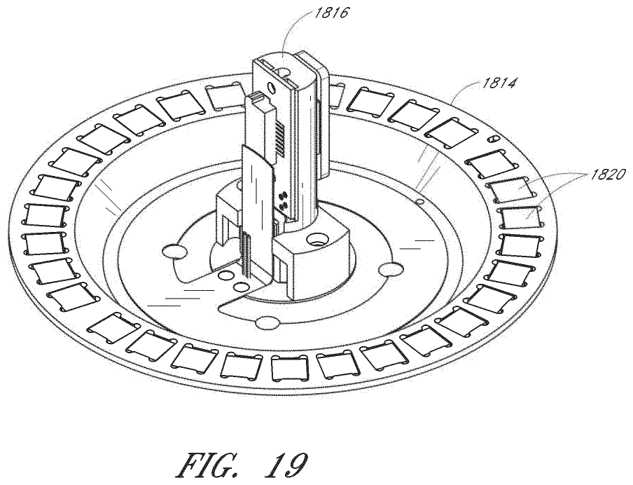

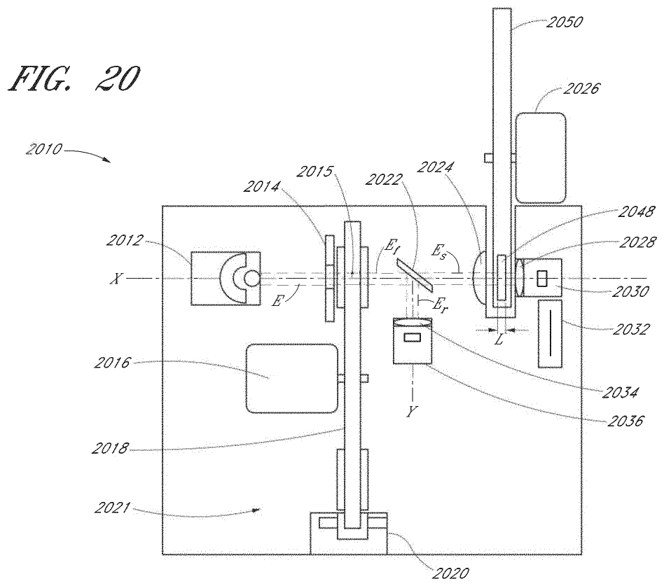

An opposite opening 1530 can provide an alternative optical pathway between a radiation source and a radiation detector (e.g., the radiation source 826 of FIG. 18) and may be used, for example, for obtaining a calibration measurement of the source and detector without an intervening window or sample. Thus, the opposite opening 1530 can be located generally at the same radial distance from the axis of rotation as the window 1556.

The receiving nubs 1512B communicate with a shunt passage 1586 inside the sample cell holder 820 (which can correspond, for example to the shunt 586 of FIG. 5).

Other features of the sample cell holder 820 can provide balancing properties for even rotation of the sample cell holder 820. For example, the wide trough 1562 and the narrower trough 1564 can be sized or otherwise configured so that the weight and/or mass of the sample cell holder 820 is evenly distributed from left to right in the view of FIG. 16, and/or from top to bottom in this view of FIG. 16.

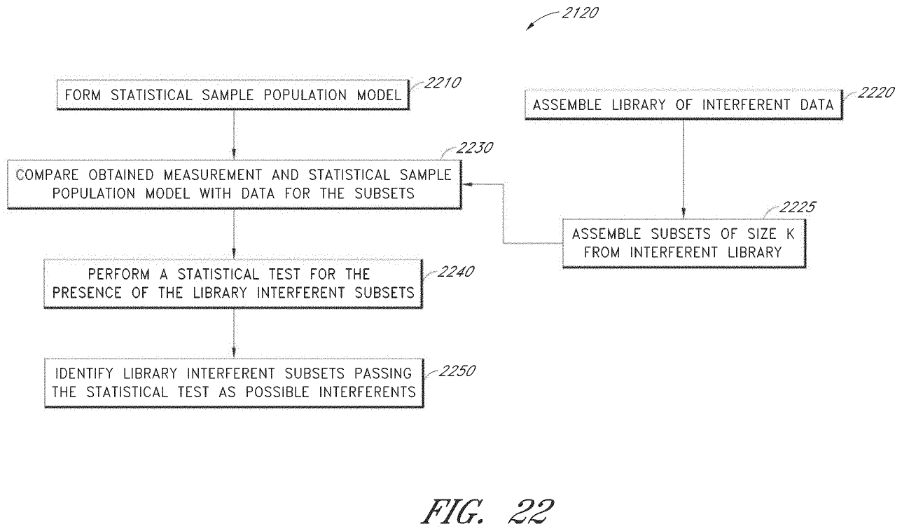

FIG. 17 shows a top perspective view of the centrifuge interface 1036 connected to the sample cell holder 820. The centrifuge interface 1036 can have a bulkhead 1520 with a rounded slot 1522 into which an actuating portion of a centrifuge can be slid from the side. The centrifuge interface 1036 can thus be spun about an axis 1524, along with the sample cell holder 820, causing fluid (e.g., whole blood) within the sample cell 1548 to separate into concentric strata, according to relative density of the fluid components (e.g., plasma, red blood cells, buffy coat, etc.), within the sample cell 1548. The sample cell holder 820 can be transparent, or it can at least have transparent portions (e.g., the window 1556 and/or the opposite opening 1530) through which radiation can pass, and which can be aligned with an optical pathway between a radiation source and a radiation detector (see, e.g., FIG. 20). In addition, a round opening 1530 through centrifuge rotor 1520 provides an optical pathway between the radiation source and radiation detector and may be used, for example, for obtaining a calibration measurement of the source and detector without an intervening window or sample.

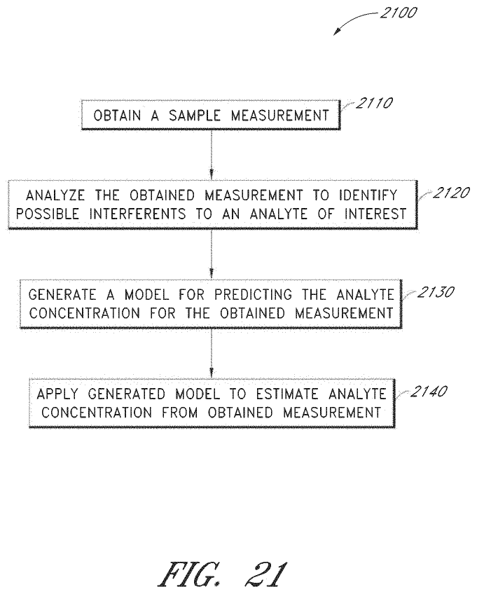

FIG. 18 shows a perspective view of an example optical system 803. Such a system can be integrated with other systems as shown in FIG. 9, for example. The optical system 803 can fill the role of the optical system 412, and it can be integrated with and/or adjacent to a fluid system (e.g., the fluid-handling system 404 or the fluid system 801). The sample cell holder 820 can be seen attached to the centrifuge interface 1036, which is in turn connected to, and rotatable by the centrifuge motor 818. A filter wheel housing 1812 is attached to the filter wheel motor 822 and encloses a filter wheel 1814. A protruding shaft assembly 1816 can be connected to the filter wheel 1814. The filter wheel 1814 can have multiple filters (see FIG. 19). The radiation source 826 is aligned to transmit radiation through a filter in the filter wheel 1814 and then through a portion of the sample cell holder 820. Transmitted and/or reflected and/or scattered radiation can then be detected by a radiation detector.

FIG. 19 shows a view of the filter wheel 1814 when it is not located within the filter wheel housing 1812 of the optical system 803. Additional features of the protruding shaft assembly 1816 can be seen, along with multiple filters 1820. In some embodiments, the filters 1820 can be removably and/or replaceably inserted into the filter wheel 1814.

### Spectroscopic System

As described above with reference to FIG. 4, the system 400 comprises the optical system 412 for analysis of a fluid sample. In various embodiments, the optical system 412 comprises one or more optical components including, for example, a spectrometer, a photometer, a reflectometer, or any other suitable device for measuring optical properties of the fluid sample. The optical system 412 may perform one or more optical measurements on the fluid sample including, for example, measurements of transmittance, absorbance, reflectance, scattering, and/or polarization. The optical measurements may be performed in one or more wavelength ranges including, for example, infrared (IR) and/or optical wavelengths. As described with reference to FIG. 4 (and further described below), the measurements from the optical system 412 are communicated to the algorithm processor 416 for analysis. For example, In some embodiments the algorithm processor 416 computes concentration of analyte(s) (and/or interferent(s)) of interest in the fluid sample. Analytes of interest include, e.g., glucose and lactate in whole blood or blood plasma.

FIG. 20 schematically illustrates an embodiment of the optical system 412 that comprises a spectroscopic analyzer 2010 adapted to measure spectra of a fluid sample such as, for example, blood or blood plasma. The analyzer 2010 comprises an energy source 2012 disposed along an optical axis X of the analyzer 2010. When activated, the energy source 2012 generates an electromagnetic energy beam E, which advances from the energy source 2012 along the optical axis X. In some embodiments, the energy source 2012 comprises an infrared energy source, and the energy beam E comprises an infrared beam. In some embodiments, the infrared energy beam E comprises a mid-infrared energy beam or a near-infrared energy beam. In some embodiments, the energy beam E can include optical and/or radio frequency wavelengths.

The energy source 2012 may comprise a broad-band and/or a narrow-band source of electromagnetic energy. In some embodiments, the energy source 2012 comprises optical elements such as, e.g., filters, collimators, lenses, mirrors, etc., that are adapted to produce a desired energy beam E. For example, in some embodiments, the energy beam E is an infrared beam in a wavelength range between about 2 μm and 20 μm. In some embodiments, the energy beam E comprises an infrared beam in a wavelength range between about 4 μm and 10 μm. In the infrared wavelength range, water generally is the main contributor to the total absorption together with features from absorption of other blood components, particularly in the 6 μm-10 μm range. The 4 μm to 10 μm wavelength band has been found to be advantageous for determining glucose concentration, because glucose has a strong absorption peak structure from about 8.5 μm to 10 μm, whereas most other blood components have a relatively low and flat absorption spectrum in the 8.5 μm to 10 μm range. Two exceptions are water and hemoglobin, which are interferents in this range.

The energy beam E may be temporally modulated to provide increased signal-to-noise ratio (S/N) of the measurements provided by the analyzer 2010 as further described below. For example, in some embodiments, the beam E is modulated at a frequency of about 10 Hz or in a range from about 1 Hz to about 30 Hz. A suitable energy source 2012 may be an electrically modulated thin-film thermoresistive element such as the HawkEye IR-50 available from Hawkeye Technologies of Milford, Conn.

As depicted in FIG. 20, the energy beam E propagates along the optical axis X and passes through an aperture 2014 and a filter 2015 thereby providing a filtered energy beam E_{f}. The aperture 2014 helps collimate the energy beam E and can include one or more filters adapted to reduce the filtering burden of the filter 2015. For example, the aperture 2014 may comprise a broadband filter that substantially attenuates beam energy outside a wavelength band between about 4 μm to about 10 μm. The filter 2015 may comprise a narrow-band filter that substantially attenuates beam energy having wavelengths outside of a filter passband (which may be tunable or user-selectable in some embodiments). The filter passband may be specified by a half-power bandwidth (“HPBW”). In some embodiments, the filter 2015 may have an HPBW in a range from about 0.1 μm to about 2 μm, or 0.01 μm to about 1 μm. In some embodiments, the bandwidths are in a range from about 0.2 μm to 0.5 μm, or 0.1 μm to 0.35 μm. Other filter bandwidths may be used. The filter 2015 may comprise a varying-passband filter, an electronically tunable filter, a liquid crystal filter, an interference filter, and/or a gradient filter. In some embodiments, the filter 2015 comprises one or a combination of a grating, a prism, a monochrometer, a Fabry-Perot etalon, and/or a polarizer. Other optical elements may be utilized as well.

In the embodiment shown in FIG. 20, the analyzer 2010 comprises a filter wheel assembly 2021 configured to dispose one or more filters 2015 along the optical axis X. The filter wheel assembly 2021 comprises a filter wheel 2018, a filter wheel motor 2016, and a position sensor 2020. The filter wheel 2018 may be substantially circular and have one or more filters 2015 or other optical elements (e.g., apertures, gratings, polarizers, mirrors, etc.) disposed around the circumference of the wheel 2018. In some embodiments, the number of filters 2015 in the filter wheel 2016 may be, for example, 1, 2, 5, 10, 15, 20, 25, or more. The motor 2016 is configured to rotate the filter wheel 2018 to dispose a desired filter 2015 (or other optical element) in the energy beam E so as to produce the filtered beam E_{f}. In some embodiments, the motor 2016 comprises a stepper motor. The position sensor 2020 determines the angular position of the filter wheel 2016, and communicates a corresponding filter wheel position signal to the algorithm processor 416, thereby indicating which filter 2015 is in position on the optical axis X. In various embodiments, the position sensor 2020 may be a mechanical, optical, and/or magnetic encoder. An alternative to the filter wheel 2018 is a linear filter translated by a motor. The linear filter can include an array of separate filters or a single filter with properties that change along a linear dimension.

The filter wheel motor 2016 rotates the filter wheel 2018 to position the filters 2015 in the energy beam E to sequentially vary the wavelengths or the wavelength bands used to analyze the fluid sample. In some embodiments, each individual filter 2015 is disposed in the energy beam E for a dwell time during which optical properties in the passband of the filter are measured for the sample. The filter wheel motor 2016 then rotates the filter wheel 2018 to position another filter 2015 in the beam E. In some embodiments, 25 narrow-band filters are used in the filter wheel 2018, and the dwell time is about 2 seconds for each filter 2015. A set of optical measurements for all the filters can be taken in about 2 minutes, including sampling time and filter wheel movement. In some embodiments, the dwell time may be different for different filters 2015, for example, to provide a substantially similar S/N ratio for each filter measurement. Accordingly, the filter wheel assembly 2021 functions as a varying-passband filter that allows optical properties of the sample to be analyzed at a number of wavelengths or wavelength bands in a sequential manner.

In some embodiments of the analyzer 2010, the filter wheel 2018 includes 25 finite-bandwidth infrared filters having a Gaussian transmission profile and full-width half-maximum (FWHM) bandwidth of 28 cm⁻¹ corresponding to a bandwidth that varies from 0.14 μm at 7.08 μm to 0.28 μm at 10 μm. The central wavelength of the filters are, in microns: 7.082, 7.158, 7.241, 7.331, 7.424, 7.513, 7.605, 7.704, 7.800, 7.905, 8.019, 8.150, 8.271, 8.598, 8.718, 8.834, 8.969, 9.099, 9.217, 9.346, 9.461, 9.579, 9.718, 9.862, and 9.990.

With further reference to FIG. 20, the filtered energy beam E_{f} propagates to a beamsplitter 2022 disposed along the optical axis X. The beamsplitter 2022 separates the filtered energy beam E_{f} into a sample beam Eₛ and a reference beam Eᵣ. The reference beam Eᵣ propagates along a minor optical axis Y, which in this embodiment is substantially orthogonal to the optical axis X. The energies in the sample beam Eₛ and the reference beam Eᵣ may comprise any suitable fraction of the energy in the filtered beam E_{f}. For example, in some embodiments, the sample beam Eₛ comprises about 80%, and the reference beam Eᵣ comprises about 20%, of the filtered beam energy E_{f}. A reference detector 2036 is positioned along the minor optical axis Y. An optical element 2034, such as a lens, may be used to focus or collimate the reference beam Eᵣ onto the reference detector 2036. The reference detector 2036 provides a reference signal, which can be used to monitor fluctuations in the intensity of the energy beam E emitted by the source 2012. Such fluctuations may be due to drift effects, aging, wear, or other imperfections in the source 2012. The algorithm processor 416 may utilize the reference signal to identify changes in properties of the sample beam Eₛ that are attributable to changes in the emission from the source 2012 and not to the properties of the fluid sample. By so doing, the analyzer 2010 may advantageously reduce possible sources of error in the calculated properties of the fluid sample (e.g., concentration). In other embodiments of the analyzer 2010, the beamsplitter 2022 is not used, and substantially all of the filtered energy beam E_{f} propagates to the fluid sample.

As illustrated in FIG. 20, the sample beam Eₛ propagates along the optical axis X, and a relay lens 2024 transmits the sample beam Eₛ into a sample cell 2048 so that at least a fraction of the sample beam Eₛ is transmitted through at least a portion of the fluid sample in the sample cell 2048. A sample detector 2030 is positioned along the optical axis X to measure the sample beam Eₛ that has passed through the portion of the fluid sample. An optical element 2028, such as a lens, may be used to focus or collimate the sample beam Eₛ onto the sample detector 2030. The sample detector 2030 provides a sample signal that can be used by the algorithm processor 416 as part of the sample analysis.

In the embodiment of the analyzer 2010 shown in FIG. 20, the sample cell 2048 is located toward the outer circumference of the centrifuge wheel 2050 (which can correspond, for example, to the sample cell holder 820 described herein). The sample cell 2048 preferably comprises windows that are substantially transmissive to energy in the sample beam Eₛ. For example, in implementations using mid-infrared energy, the windows may comprise calcium fluoride. As described herein with reference to FIG. 5, the sample cell 2048 is in fluid communication with an injector system that permits filling the sample cell 2048 with a fluid sample (e.g., whole blood) and flushing the sample cell 2048 (e.g., with saline or a detergent). The injector system may disconnect after filling the sample cell 2048 with the fluid sample to permit free spinning of the centrifuge wheel 2050.

The centrifuge wheel 2050 can be spun by a centrifuge motor 2026. In some embodiments of the analyzer 2010, the fluid sample (e.g., a whole blood sample) is spun at a certain number of revolutions per minute (RPM) for a given length of time to separate blood plasma for spectral analysis. In some embodiments, the fluid sample is spun at about 7200 RPM. In some embodiments, the fluid sample is spun at about 5000 RPM or 4500 RPM. In some embodiments, the fluid sample is spun at more than one rate for successive time periods. The length of time can be approximately 5 minutes. In some embodiments, the length of time is approximately 2 minutes. In some embodiments, an anti-clotting agent such as heparin may be added to the fluid sample before centrifuging to reduce clotting. With reference to FIG. 20, the centrifuge wheel 2050 is rotated to a position where the sample cell 2048 intercepts the sample beam Eₛ, allowing energy to pass through the sample cell 2048 to the sample detector 2030.

The embodiment of the analyzer 2010 illustrated in FIG. 20 advantageously permits direct measurement of the concentration of analytes in the plasma sample rather than by inference of the concentration from measurements of a whole blood sample. An additional advantage is that relatively small volumes of fluid may be spectroscopically analyzed. For example, in some embodiments the fluid sample volume is between about 1 μL and 80 μL and is about 25 μL in some embodiments. In some embodiments, the sample cell 2048 is disposable and is intended for use with a single patient or for a single measurement.

In some embodiments, the reference detector 2036 and the sample detector 2030 comprise broadband pyroelectric detectors. As known in the art, some pyroelectric detectors are sensitive to vibrations. Thus, for example, the output of a pyroelectric infrared detector is the sum of the exposure to infrared radiation and to vibrations of the detector. The sensitivity to vibrations, also known as “microphonics,” can introduce a noise component to the measurement of the reference and sample energy beams Eᵣ, Eₛ using some pyroelectric infrared detectors. Because it may be desirable for the analyzer 2010 to provide high signal-to-noise ratio measurements, such as, e.g., S/N in excess of 100 dB, some embodiments of the analyzer 2010 utilize one or more vibrational noise reduction apparatus or methods. For example, the analyzer 2010 may be mechanically isolated so that high S/N spectroscopic measurements can be obtained for vibrations below an acceleration of about 1.5 G.

In some embodiments of the analyzer 2010, vibrational noise can be reduced by using a temporally modulated energy source 2012 combined with an output filter. In some embodiments, the energy source 2012 is modulated at a known source frequency, and measurements made by the detectors 2036 and 2030 are filtered using a narrowband filter centered at the source frequency. For example, in some embodiments, the energy output of the source 2012 is sinusoidally modulated at 10 Hz, and outputs of the detectors 2036 and 2030 are filtered using a narrow bandpass filter of less than about 1 Hz centered at 10 Hz. Accordingly, microphonic signals that are not at 10 Hz are significantly attenuated. In some embodiments, the modulation depth of the energy beam E may be greater than 50% such as, for example, 80%. The duty cycle of the beam may be between about 30% and 70%. The temporal modulation may be sinusoidal or any other waveform. In embodiments utilizing temporally modulated energy sources, detector output may be filtered using a synchronous demodulator and digital filter. The demodulator and filter are software components that may be digitally implemented in a processor such as the algorithm processor 416. Synchronous demodulators, coupled with low pass filters, are often referred to as “lock in amplifiers.”

The analyzer 2010 may also include a vibration sensor 2032 (e.g., one or more accelerometers) disposed near one (or both) of the detectors 2036 and 2030. The output of the vibration sensor 2032 is monitored, and suitable actions are taken if the measured vibration exceeds a vibration threshold. For example, in some embodiments, if the vibration sensor 2032 detects above-threshold vibrations, the system discards any ongoing measurement and “holds off” on performing further measurements until the vibrations drop below the threshold. Discarded measurements may be repeated after the vibrations drop below the vibration threshold. In some embodiments, if the duration of the “hold off” is sufficiently long, the fluid in the sample cell 2030 is flushed, and a new fluid sample is delivered to the cell 2030 for measurement. The vibration threshold may be selected so that the error in analyte measurement is at an acceptable level for vibrations below the threshold. In some embodiments, the threshold corresponds to an error in glucose concentration of 5 mg/dL. The vibration threshold may be determined individually for each filter 2015.

Certain embodiments of the analyzer 2010 include a temperature system (not shown in FIG. 20) for monitoring and/or regulating the temperature of system components (such as the detectors 2036, 2030) and/or the fluid sample. Such a temperature system can include temperature sensors, thermoelectrical heat pumps (e.g., a Peltier device), and/or thermistors, as well as a control system for monitoring and/or regulating temperature. In some embodiments, the control system comprises a proportional-plus-integral-plus-derivative (PID) control. For example, in some embodiments, the temperature system is used to regulate the temperature of the detectors 2030, 2036 to a desired operating temperature, such as 35 degrees Celsius.

### Optical Measurement

The analyzer 2010 illustrated in FIG. 20 can be used to determine optical properties of a substance in the sample cell 2048. The substance can include whole blood, plasma, saline, water, air or other substances. In some embodiments, the optical properties include measurements of an absorbance, transmittance, and/or optical density in the wavelength passbands of some or all of the filters 2015 disposed in the filter wheel 2018. As described above, a measurement cycle comprises disposing one or more filters 2015 in the energy beam E for a dwell time and measuring a reference signal with the reference detector 2036 and a sample signal with the sample detector 2030. The number of filters 2015 used in the measurement cycle will be denoted by N, and each filter 2015 passes energy in a passband around a center wavelength λᵢ, where i is an index ranging over the number of filters (e.g., from 1 to N). The set of optical measurements from the sample detector 2036 in the passbands of the N filters 2015 provide a wavelength-dependent spectrum of the substance in the sample cell 2048. The spectrum will be denoted by Cₛ(λᵢ), where Cₛ may be a transmittance, absorbance, optical density, or some other measure of an optical property of the substance. In some embodiments, the spectrum is normalized with respect to one or more of the reference signals measured by the reference detector 2030 and/or with respect to spectra of a reference substance (e.g., air or saline). The measured spectra are communicated to the algorithm processor 416 for calculation of the concentration of the analyte(s) of interest in the fluid sample.

In some embodiments, the analyzer 2010 performs spectroscopic measurements on the fluid sample (known as a “wet” reading) and on one or more reference samples. For example, an “air” reading occurs when the sample detector 2036 measures the sample signal without the sample cell 2048 in place along the optical axis X. (This can occur, for example, when the opposite opening 1530 is aligned with the optical axis X). A “water” or “saline” reading occurs when the sample cell 2048 is filled with water or saline, respectively. The algorithm processor 416 may be programmed to calculate analyte concentration using a combination of these spectral measurements.

In some embodiments, a pathlength corrected spectrum is calculated using wet, air, and reference readings. For example, the transmittance at wavelength λᵢ, denoted by Tᵢ, may be calculated according to Tᵢ=(Sᵢ(wet)/Rᵢ(wet))/(Sᵢ(air)/Rᵢ(air)), where Sᵢ denotes the sample signal from the sample detector 2036 and Rᵢ denotes the corresponding reference signal from the reference detector 2030. In some embodiments, the algorithm processor 416 calculates the optical density, ODᵢ, as a logarithm of the transmittance, e.g., according to ODᵢ=−Log(Tᵢ). In one implementation, the analyzer 2010 takes a set of wet readings in each of the N filter passbands and then takes a set of air readings in each of the N filter passbands. In other embodiments, the analyzer 2010 may take an air reading before (or after) the corresponding wet reading.

The optical density ODᵢ is the product of the absorption coefficient at wavelength λᵢ, αᵢ, times the pathlength L over which the sample energy beam Eₛ interacts with the substance in the sample cell 2048, e.g., ODᵢ=αᵢ L. The absorption coefficient αᵢ of a substance may be written as the product of an absorptivity per mole times a molar concentration of the substance. FIG. 20 schematically illustrates the pathlength L of the sample cell 2048. The pathlength L may be determined from spectral measurements made when the sample cell 2048 is filled with a reference substance. For example, because the absorption coefficient for water (or saline) is known, one or more water (or saline) readings can be used to determine the pathlength L from measurements of the transmittance (or optical density) through the cell 2048. In some embodiments, several readings are taken in different wavelength passbands, and a curve-fitting procedure is used to estimate a best-fit pathlength L. The pathlength L may be estimated using other methods including, for example, measuring interference fringes of light passing through an empty sample cell 2048.

The pathlength L may be used to determine the absorption coefficients of the fluid sample at each wavelength. Molar concentration of an analyte of interest can be determined from the absorption coefficient and the known molar absorptivity of the analyte. In some embodiments, a sample measurement cycle comprises a saline reading (at one or more wavelengths), a set of N wet readings (taken, for example, through a sample cell 2048 containing saline solution), followed by a set of N air readings (taken, for example, through the opposite opening 1530). As discussed above, the sample measurement cycle can be performed in a given length of time that may depend, at least in part, on filter dwell times. For example, the measurement cycle may take five minutes when the filter dwell times are about five seconds. In some embodiments, the measurement cycle may take about two minutes when the filter dwell times are about two seconds. After the sample measurement cycle is completed, a detergent cleaner may be flushed through the sample cell 2048 to reduce buildup of organic matter (e.g., proteins) on the windows of the sample cell 2048. The detergent is then flushed to a waste bladder.

In some embodiments, the system stores information related to the spectral measurements so that the information is readily available for recall by a user. The stored information can include wavelength-dependent spectral measurements (including fluid sample, air, and/or saline readings), computed analyte values, system temperatures and electrical properties (e.g., voltages and currents), and any other data related to use of the system (e.g., system alerts, vibration readings, S/N ratios, etc.). The stored information may be retained in the system for a time period such as, for example, 30 days. After this time period, the stored information may be communicated to an archival data storage system and then deleted from the system. In some embodiments, the stored information is communicated to the archival data storage system via wired or wireless methods, e.g., over a hospital information system (HIS).

### Analyte Analysis

The algorithm processor 416 (FIG. 4) (or any other suitable processor or processors) may be configured to receive from the analyzer 2010 the wavelength-dependent optical measurements Cs(λᵢ) of the fluid sample. In some embodiments, the optical measurements comprise spectra such as, for example, optical densities ODᵢ measured in each of the N filter passbands centered around wavelengths λᵢ. The optical measurements Cs(λᵢ) are communicated to the processor 416, which analyzes the optical measurements to detect and quantify one or more analytes in the presence of interferents. In some embodiments, one or more poor quality optical measurements Cs(λᵢ) are rejected (e.g., as having a S/N ratio that is too low), and the analysis performed on the remaining, sufficiently high-quality measurements. In another embodiment, additional optical measurements of the fluid sample are taken by the analyzer 2010 to replace one or more of the poor quality measurements.

Interferents can comprise components of a material sample being analyzed for an analyte, where the presence of the interferent affects the quantification of the analyte. Thus, for example, in the spectroscopic analysis of a sample to determine an analyte concentration, an interferent could be a compound having spectroscopic features that overlap with those of the analyte, in at least a portion of the wavelength range of the measurements. The presence of such an interferent can introduce errors in the quantification of the analyte. More specifically, the presence of one or more interferents can affect the sensitivity of a measurement technique to the concentration of analytes of interest in a material sample, especially when the system is calibrated in the absence of, or with an unknown amount of, the interferent.

Independently of or in combination with the attributes of interferents described above, interferents can be classified as being endogenous (i.e., originating within the body) or exogenous (i.e., introduced from or produced outside the body). As an example of these classes of interferents, consider the analysis of a blood sample (or a blood component sample or a blood plasma sample) for the analyte glucose. Endogenous interferents include those blood components having origins within the body that affect the quantification of glucose, and can include water, hemoglobin, blood cells, and any other component that naturally occurs in blood. Exogenous interferents include those blood components having origins outside of the body that affect the quantification of glucose, and can include items administered to a person, such as medicaments, drugs, foods or herbs, whether administered orally, intravenously, topically, etc.

Independently of or in combination with the attributes of interferents described above, interferents can comprise components which are possibly, but not necessarily, present in the sample type under analysis. In the example of analyzing samples of blood or blood plasma drawn from patients who are receiving medical treatment, a medicament such as acetaminophen is possibly, but not necessarily, present in this sample type. In contrast, water is necessarily present in such blood or plasma samples.

Certain disclosed analysis methods are particularly effective if each analyte and interferent has a characteristic signature in the measurement (e.g., a characteristic spectroscopic feature), and if the measurement is approximately affine (e.g., includes a linear term and an offset) with respect to the concentration of each analyte and interferent. In such methods, a calibration process is used to determine a set of one or more calibration coefficients and a set of one or more optional offset values that permit the quantitative estimation of an analyte. For example, the calibration coefficients and the offsets may be used to calculate an analyte concentration from spectroscopic measurements of a material sample (e.g., the concentration of glucose in blood plasma). In some of these methods, the concentration of the analyte is estimated by multiplying the calibration coefficient by a measurement value (e.g., an optical density) to estimate the concentration of the analyte. Both the calibration coefficient and measurement can comprise arrays of numbers. For example, in some embodiments, the measurement comprises spectra Cₛ(λᵢ) measured at the wavelengths λᵢ, and the calibration coefficient and optional offset comprise an array of values corresponding to each wavelength λᵢ. In some embodiments, as further described below, a hybrid linear analysis (HLA) technique is used to estimate analyte concentration in the presence of a set of interferents, while retaining a high degree of sensitivity to the desired analyte. The data used to accommodate the set of possible interferents can include (a) signatures of each of the members of the family of potential additional substances and (b) a typical quantitative level at which each additional substance, if present, is likely to appear. In some embodiments, the calibration coefficient (and optional offset) are adjusted to minimize or reduce the sensitivity of the calibration to the presence of interferents that are identified as possibly being present in the fluid sample.

In some embodiments, the analyte analysis method uses a set of training spectra each having known analyte concentration and produces a calibration that minimizes the variation in estimated analyte concentration with interferent concentration. The resulting calibration coefficient indicates sensitivity of the measurement to analyte concentration. The training spectra need not include a spectrum from the individual whose analyte concentration is to be determined. That is, the term “training” when used in reference to the disclosed methods does not require training using measurements from the individual whose analyte concentration will be estimated (e.g., by analyzing a bodily fluid sample drawn from the individual).

Several terms are used herein to describe the analyte analysis process. The term “Sample Population” is a broad term and includes, without limitation, a large number of samples having measurements that are used in the computation of calibration values (e.g., calibration coefficients and optional offsets). In some embodiments, the term Sample Population comprises measurements (such as, e.g., spectra) from individuals and may comprise one or more analyte measurements determined from those same individuals. Additional demographic information may be available for the individuals whose sample measurements are included in the Sample Population. For an embodiment involving the spectroscopic determination of glucose concentration, the Sample Population measurements may include a spectrum (measurement) and a glucose concentration (analyte measurement).

Various embodiments of Sample Populations may be used in various embodiments of the systems and methods described herein. Several examples of Sample Populations will now be described. These examples are intended to illustrate certain aspects of possible Sample Population embodiments but are not intended to limit the types of Sample Populations that may be generated. In certain embodiments, a Sample Population may include samples from one or more of the example Sample Populations described below.

In some embodiments of the systems and methods described herein, one or more Sample Populations are included in a “Population Database.” The Population Database may be implemented and/or stored on a computer-readable medium. In certain embodiments, the systems and methods may access the Population Database using wired and/or wireless techniques. Certain embodiments may utilize several different Population Databases that are accessible locally and/or remotely. In some embodiments, the Population Database includes one or more of the example Sample Populations described below. In some embodiments, two or more databases can be combined into a single database, and in other embodiments, any one database can be divided into multiple databases.

An example Sample Population may comprise samples from individuals belonging to one or more demographic groups including, for example, ethnicity, nationality, gender, age, etc. Demographic groups may be established for any suitable set of one or more distinctive factors for the group including, for example, medical, cultural, behavioral, biological, geographical, religious, and genealogical traits. For example, in certain embodiments, a Sample Population includes samples from individuals from a specific ethnic group (e.g., Caucasians, Hispanics, Asians, African Americans, etc.). In another embodiment, a Sample Population includes samples from individuals of a specific gender or a specific race. In some embodiments, a Sample Population includes samples from individuals belonging to more than one demographic group (e.g., samples from Caucasian women).

Another example Sample Population can comprise samples from individuals having one or more medical conditions. For example, a Sample Population may include samples from individuals who are healthy and unmedicated (sometimes referred to as a Normal Population). In some embodiments, the Sample Population includes samples from individuals having one or more health conditions (e.g., diabetes). In some embodiments, the Sample Population includes samples from individuals taking one or more medications. In certain embodiments, Sample Population includes samples from individuals diagnosed to have a certain medical condition or from individuals being treated for certain medical conditions or some combination thereof. The Sample Population may include samples from individuals such as, for example, ICU patients, maternity patients, and so forth.

An example Sample Population may comprise samples that have the same interferent or the same type of interferents. In some embodiments, a Sample Population can comprise multiple samples, all lacking an interferent or a type of interferent. For example, a Sample Population may comprise samples that have no exogenous interferents, that have one or more exogenous interferents of either known or unknown concentration, and so forth. The number of interferents in a sample depends on the measurement and analyte(s) of interest, and may number, in general, from zero to a very large number (e.g., greater than 300). All of the interferents typically are not expected to be present in a particular material sample, and in many cases, a smaller number of interferents (e.g., 0, 1, 2, 5, 10, 15, 20, or 25) may be used in an analysis. In certain embodiments, the number of interferents used in the analysis is less than or equal to the number of wavelength-dependent measurements N in the spectrum Cs(λᵢ).

Certain embodiments of the systems and methods described herein are capable of analyzing a material sample using one or more Sample Populations (e.g., accessed from the Population Database). Certain such embodiments may use information regarding some or all of the interferents which may or may not be present in the material sample. In some embodiments, a list of one or more possible interferents, referred to herein as forming a “Library of Interferents,” can be compiled. Each interferent in the Library can be referred to as a “Library Interferent.” The Library Interferents may include exogenous interferents and endogenous interferents that may be present in a material sample. For example, an interferent may be present due to a medical condition causing abnormally high concentrations of the exogenous and endogenous interferents. In some embodiments, the Library of Interferents may not include one or more interferents that are known to be present in all samples. Thus, for example, water, which is a glucose interferent for many spectroscopic measurements, may not be included in the Library of Interferents. In certain embodiments, the systems and methods use samples in the Sample Population to train calibration methods.

The material sample being measured, for example a fluid sample in the sample cell 2048, may also include one or more Library Interferents which may include, but is not limited to, an exogenous interferent or an endogenous interferent. Examples of exogenous interferent can include medications, and examples of endogenous interferents can include urea in persons suffering from renal failure. In addition to components naturally found in the blood, the ingestion or injection of some medicines or illicit drugs can result in very high and rapidly changing concentrations of exogenous interferents.

In some embodiments, measurements of a material sample (e.g., a bodily fluid sample), samples in a Sample Population, and the Library Interferents comprise spectra (e.g., infrared spectra). The spectra obtained from a sample and/or an interferent may be temperature dependent. In some embodiments, it may be beneficial to calibrate for temperatures of the individual samples in the Sample Population or the interferents in the Library of Interferents. In some embodiments, a temperature calibration procedure is used to generate a temperature calibration factor that substantially accounts for the sample temperature. For example, the sample temperature can be measured, and the temperature calibration factor can be applied to the Sample Population and/or the Library Interferent spectral data. In some embodiments, a water or saline spectrum is subtracted from the sample spectrum to account for temperature effects of water in the sample.

In other embodiments, temperature calibration may not be used. For example, if Library Interferent spectra, Sample Population spectra, and sample spectra are obtained at approximately the same temperature, an error in a predicted analyte concentration may be within an acceptable tolerance. If the temperature at which a material sample spectrum is measured is within, or near, a temperature range (e.g., several degrees Celsius) at which the plurality of Sample Population spectra are obtained, then some analysis methods may be relatively insensitive to temperature variations. Temperature calibration may optionally be used in such analysis methods.

### Systems and Methods for Estimating Analyte Concentration in the Presence of Interferents

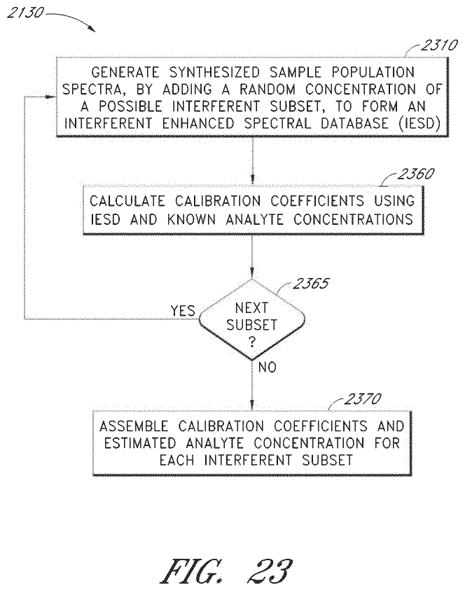

FIG. 21 is a flowchart that schematically illustrates an embodiment of a method 2100 for estimating the concentration of an analyte in the presence of interferents. In block 2110, a measurement of a sample is obtained, and in block 2120 data relating to the obtained measurement is analyzed to identify possible interferents to the analyte. In block 2130, a model is generated for predicting the analyte concentration in the presence of the identified possible interferents, and in block 2140 the model is used to estimate the analyte concentration in the sample from the measurement. In certain embodiments of the method 2100, the model generated in block 2130 is selected to reduce or minimize the effect of identified interferents that are not present in a general population of which the sample is a member.

An example embodiment of the method 2100 of FIG. 21 for the determination of an analyte (e.g., glucose) in a blood sample will now be described. This example embodiment is intended to illustrate various aspects of the method 2100 but is not intended as a limitation on the scope of the method 2100 or on the range of possible analytes. In this example, the sample measurement in block 2110 is an absorption spectrum, Cs(λᵢ), of a measurement sample S that has, in general, one analyte of interest, glucose, and one or more interferents.

In block 2120, a statistical comparison of the absorption spectrum of the sample S with a spectrum of the Sample Population and combinations of individual Library Interferent spectra is performed. The statistical comparison provides a list of Library Interferents that are possibly contained in sample S and can include either no Library Interferents or one or more Library Interferents. In this example, in block 2130, one or more sets of spectra are generated from spectra of the Sample Population and their respective known analyte concentrations and known spectra of the Library Interferents identified in block 2120. In block 2130, the generated spectra are used to calculate a model for predicting the analyte concentration from the obtained measurement. In some embodiments, the model comprises one or more calibration coefficients κ(λᵢ) that can be used with the sample measurements Cs(λᵢ) to provide an estimate of the analyte concentration, gₑₛₜ. In block 2140, the estimated analyte concentration is determined form the model generated in block 2130. For example, in some embodiments of HLA, the estimated analyte concentration is calculated according to a linear formula: gₑₛₜ=κ(λᵢ)·Cₛ(λᵢ). Because the absorption measurements and calibration coefficients may represent arrays of numbers, the multiplication operation indicated in the preceding formula may comprise a sum of the products of the measurements and coefficients (e.g., an inner product or a matrix product). In some embodiments, the calibration coefficient is determined so as to have reduced or minimal sensitivity to the presence of the identified Library Interferents.

An example embodiment of block 2120 of the method 2100 will now be described with reference to FIG. 22. In this example, block 2120 includes forming a statistical Sample Population model (block 2210), assembling a library of interferent data (block 2220), assembling all subsets of size K of the library interferents (block 2225), comparing the obtained measurement and statistical Sample Population model with data for each set of interferents from an interferent library (block 2230), performing a statistical test for the presence of each interferent from the interferent library (block 2240), and identifying possible interferents that pass the statistical test (block 2250). The size K of the subsets may be an integer such as, for example, 1, 2, 3, 4, 5, 6, 10, 16, or more. The acts of block 2220 can be performed once or can be updated as necessary. In certain embodiments, the acts of blocks 2230, 2240, and 2250 are performed sequentially for all subsets of Library Interferents that pass the statistical test (block 2240). In this example, in block 2210, a Sample Population Database is formed that includes a statistically large Sample Population of individual spectra taken over the same wavelength range as the sample spectrum, Cₛ(λᵢ). The Database also includes an analyte concentration corresponding to each spectrum. For example, if there are P Sample Population spectra, then the spectra in the Database can be represented as C={C₁, C₂, . . . , C_{P}}, and the analyte concentration corresponding to each spectrum can be represented as g={g₁, g₂, . . . , g_{P}}. In some embodiments, the Sample Population does not have any of the Library Interferents present, and the material sample has interferents contained in the Sample Population and one or more of the Library Interferents.

In some embodiments of block 2210, the statistical sample model comprises a mean spectrum and a covariance matrix calculated for the Sample Population. For example, if each spectrum measured at N wavelengths λᵢ is represented by an N×1 array, C, then the mean spectrum, μ, is an N×1 array having values at each wavelength averaged over the range of spectra in the Sample Population. The covariance matrix, V, is calculated as the expected value of the deviation between C and μ and can be written as V=E((C−μ) (C−μ)^{T}) where E(⋅) represents the expected value and the superscript T denotes transpose. In other embodiments, additional statistical parameters may be included in the statistical model of the Sample Population spectra.

Additionally, a Library of Interferents may be assembled in block 2220. A number of possible interferents can be identified, for example, as a list of possible medications or foods that might be ingested by the population of patients at issue. Spectra of these interferents can be obtained, and a range of expected interferent concentrations in the blood, or other expected sample material, can be estimated. In certain embodiments, the Library of Interferents includes, for each of “M” interferents, the absorption spectrum normalized to unit interferent concentration of each interferent, IF={IF₁, IF₂, . . . , IF_{M}}, and a range of concentrations for each interferent from Tmax={Tmax₁, Tmax₂, . . . , Tmax_{M}) to Tmin={Tmin₁, Tmin₂, . . . , Tmin_{M}). Information in the Library may be assembled once and accessed as needed. For example, the Library and the statistical model of the Sample Population may be stored in a storage device associated with the algorithm processor 416 (see, FIG. 4).

Continuing in block 2225, the algorithm processor 416 assembles one or more subsets comprising a number K of spectra taken from the Library of Interferents. The number K may be an integer such as, for example, 1, 2, 3, 4, 5, 6, 10, 16, or more. In some embodiments, the subsets comprise all combinations of the M Library spectra taken K at a time. In these embodiments, the number of subsets having K spectra is M!/(K!(M−K)!), where ! represents the factorial function.

Continuing in block 2230, the obtained measurement data (e.g., the sample spectrum) and the statistical Sample Population model (e.g., the mean spectrum and the covariance matrix) are compared with data for each subset of interferents determined in block 2225 in order to determine the presence of possible interferents in the sample (block 2240). In some embodiments, the statistical test for the presence of an interferent subset in block 2240 comprises determining the concentrations of each subset of interferences that minimize a statistical measure of “distance” between a modified spectrum of the material sample and the statistical model of the Sample Population (e.g., the mean μ and the covariance V). The term “concentration” used in this context refers to a computed value, and, in some embodiments, that computed value may not correspond to an actual concentration. The concentrations may be calculated numerically. In some embodiments, the concentrations are calculated by algebraically solving a set of linear equations. The statistical measure of distance may comprise the well-known Mahalanobis distance (or square of the Mahalanobis distance) and/or some other suitable statistical distance metric (e.g., Hotelling's T-square statistic). In certain implementations, the modified spectrum is given by C′ₛ(T)=Cₛ−IF·T where T=(T₁, T₂, . . . T_{K})^{T} is a K-dimensional column vector of interferent concentrations and IF={IF₁, IF₂, . . . IF_{K}} represents the K interferent absorption spectra of the subset. In some embodiments, concentration of the i^{th} interferent is assumed to be in a range from a minimum value, Tminᵢ, to a maximum value, Tmaxᵢ. The value of Tminᵢ may be zero, or may be a value between zero and Tmaxᵢ, such as a fraction of Tmaxᵢ, or may be a negative value. Negative values represent interferent concentrations that are smaller than baseline interferent values in the Sample Population.

In block 2250, a list of a number N_{S} of possible interferent subsets ξ may be identified as the particular subsets that pass one or more statistical tests (in block 2240) for being present in the material sample. One or more statistical tests may be used, alone or in combination, to identify the possible interferents. For example, if a statistical test indicates that an i^{th} interferent is present in a concentration outside the range Tminᵢ to Tmaxᵢ, then this result may be used to exclude the i^{th} interferent from the list of possible interferents. In some embodiments, only the single most probable interferent subset is included on the list, for example, the subset having the smallest statistical distance (e.g., Mahalanobis distance). In an embodiment, the list includes the subsets ξ having statistical distances smaller than a threshold value. In certain embodiments, the list includes a number N_{S} of subsets having the smallest statistical distances, e.g., the list comprises the “best” candidate subsets. The number N_{S} may be any suitable integer such as 10, 20, 50, 100, 200, or more. An advantage of selecting the “best” N_{S} subsets is reduced computational burden on the algorithm processor 416. In some embodiments, the list includes all the Library Interferents. In certain such embodiments, the list is selected to comprise combinations of the N_{S} subsets taken L at a time. For example, in some embodiments, pairs of subsets are taken (e.g., L=2). An advantage of selecting pairs of subsets is that pairing captures the most likely combinations of interferents and the “best” candidates are included multiple times in the list of possible interferents. In embodiments in which combinations of L subsets are selected, the number of combinations of subsets in the list of possible interferent subsets is N_{S}!/(L!(N_{S}−L)!).

In other embodiments, the list of possible interferent subsets ξ is determined using a combination of some or all of the above criteria. In another embodiment, the list of possible interferent subsets ξ includes each of the subsets assembled in block 2225. Many selection criteria are possible for the list of possible interferent subsets ξ.

Returning to FIG. 21, the method 2100 continues in block 2130 where analyte concentration is estimated in the presence of the possible interferent subsets ξ determined in block 2250. FIG. 23 is a flowchart that schematically illustrates an example embodiment of the acts of block 2130. In block 2310, synthesized Sample Population measurements are generated to form an Interferent Enhanced Spectral Database (IESD). In block 2360, the IESD and known analyte concentrations are used to generate calibration coefficients for the selected interferent subset. As indicated in block 2365, blocks 2310 and 2360 may be repeated for each interferent subset ξ identified in the list of possible interferent subsets (e.g., in block 2250 of FIG. 22). In this example embodiment, when all the interferent subsets ξ have been processed, the method continues in block 2370, wherein an average calibration coefficient is applied to the measured spectra to determine a set of analyte concentrations.

In one example embodiment for block 2310, synthesized Sample Population spectra are generated by adding random concentrations of each interferent in one of the possible interferent subsets ξ. These spectra are referred to herein as an Interferent-Enhanced Spectral Database or IESD. In one example method, the IESD is formed as follows. A plurality of Randomly-Scaled Single Interferent Spectra (RSIS) are formed for each interferent in the interferent subset ξ. Each RSIS is formed by combinations of the interferent having spectrum IF multiplied by the maximum concentration Tmax, which is scaled by a random factor between zero and one. In certain embodiments, the scaling places the maximum concentration at the 95^{th} percentile of a log-normal distribution in order to generate a wide range of concentrations. In some embodiments, the log-normal distribution has a standard deviation equal to half of its mean value.

In this example method, individual RSIS are then combined independently and in random combinations to form a large family of Combination Interferent Spectra (CIS), with each spectrum in the CIS comprising a random combination of RSIS, selected from the full set of identified Library Interferents. An advantage of this method of selecting the CIS is that it produces adequate variability with respect to each interferent, independently across separate interferents.

The CIS and replicates of the Sample Population spectra are combined to form the IESD. Since the interferent spectra and the Sample Population spectra may have been obtained from measurements having different optical pathlengths, the CIS may be scaled to the same pathlength as the Sample Population spectra. The Sample Population Database is then replicated R times, where R depends on factors including the size of the Database and the number of interferents. The IESD includes R copies of each of the Sample Population spectra, where one copy is the original Sample Population Data, and the remaining R-1 copies each have one randomly chosen CIS spectra added. Accordingly, each of the IESD spectra has an associated analyte concentration from the Sample Population spectra used to form the particular IESD spectrum. In some embodiments, a 10-fold replication of the Sample Population Database is used for 130 Sample Population spectra obtained from 58 different individuals and 18 Library Interferents. A smaller replication factor may be used if there is greater spectral variety among the Library Interferent spectra, and a larger replication factor may be used if there is a greater number of Library Interferents.

After the IESD is generated in block 2310, in block 2360, the IESD spectra and the known, random concentrations of the subset interferents are used to generate a calibration coefficient for estimating the analyte concentration from a sample measurement. The calibration coefficient is calculated in some embodiments using a hybrid linear analysis (HLA) technique. In certain embodiments, the HLA technique uses a reference analyte spectrum to construct a set of spectra that are free of the desired analyte, projecting the analyte's spectrum orthogonally away from the space spanned by the analyte-free calibration spectra, and normalizing the result to produce a unit response. Further description of embodiments of HLA techniques may be found in, for example, “Measurement of Analytes in Human Serum and Whole Blood Samples by Near-Infrared Raman Spectroscopy,” Chapter 4, Andrew J. Berger, Ph. D. thesis, Massachusetts Institute of Technology, 1998, and “An Enhanced Algorithm for Linear Multivariate Calibration,” by Andrew J. Berger, et al., Analytical Chemistry, Vol. 70, No. 3, Feb. 1, 1998, pp. 623-627, the entirety of each of which is hereby incorporated by reference herein. In other embodiments, the calibration coefficients may be calculated using other techniques including, for example, regression techniques such as, for example, ordinary least squares (OLS), partial least squares (PLS), and/or principal component analysis.

In block 2365, the processor 416 determines whether additional interferent subsets ξ remain in the list of possible interferent subsets. If another subset is present in the list, the acts in blocks 2310-2360 are repeated for the next subset of interferents using different random concentrations. In some embodiments, blocks 2310-2360 are performed for only the most probable subset on the list.

The calibration coefficient determined in block 2360 corresponds to a single interferent subset ξ from the list of possible interferent subsets and is denoted herein as a single-interferent-subset calibration coefficient κ_{avg}(ξ). In this example method, after all subsets ξ have been processed, the method continues in block 2370, in which the single-interferent-subset calibration coefficient is applied to the measured spectra Cₛ to determine an estimated, single-interferent-subset analyte concentration, g(ξ)=κ_{avg}(ξ)·Cₛ, for the interferent subset ξ. The set of the estimated, single-interferent-subset analyte concentrations g(ξ) for all subsets in the list may be assembled into an array of single-interferent-subset concentrations. As noted above, in some embodiments the blocks 2310-2370 are performed once for the most probable single-interferent-subset on the list (e.g., the array of single-interferent analyte concentrations has a single member).

Returning to block 2140 of FIG. 21, the array of single-interferent-subset concentrations, g(ξ), is combined to determine an estimated analyte concentration, gₑₛₜ, for the material sample. In certain embodiments, a weighting function p(ξ) is determined for each of the interferent subsets ξ on the list of possible interferent subsets. The weighting functions may be normalized such that Σp(ξ)=1, where the sum is over all subsets ξ that have been processed from the list of possible interferent subsets. In some embodiments, the weighting functions can be related to the minimum Mahalanobis distance or an optimal concentration. In certain embodiments, the weighting function p(ξ), for each subset ξ, is selected to be a constant, e.g., 1/N_{S} where N_{S} is the number of subsets processed from the list of possible interferent subsets. In other embodiments, other weighting functions p(ξ) can be selected.

In certain embodiments, the estimated analyte concentration, gₑₛₜ, is determined (in block 2140) by combining the single-interferent-subset estimates, g(ξ), and the weighting functions, p(ξ), to generate an average analyte concentration. The average concentration may be computed according to gₑₛₜ=E g(ξ) p(ξ), where the sum is over the interferent subsets processed from the list of possible interferent subsets. In some embodiments, the weighting function p(ξ) is a constant value for each subset (e.g., a standard arithmetic average is used for determining average analyte concentration). By testing the above described example method on simulated data, it has been found that the average analyte concentration advantageously has errors that may be reduced in comparison to other methods (e.g., methods using only a single most probable interferent).

Although the flowchart in FIG. 21 schematically illustrates an embodiment of the method 2100 performed with reference to the blocks 2110-2140 described herein, in other embodiments, the method 2100 can be performed differently. For example, some or all of the blocks 2110-2140 can be combined, performed in a different order than shown, and/or the functions of particular blocks may be reallocated to other blocks and/or to different blocks. Embodiments of the method 2100 may utilize different blocks than are shown in FIG. 21.

For example, in some embodiments of the method 2100, the calibration coefficient is computed without synthesizing spectra and/or partitioning the data into calibration sets and test sets. Such embodiments are referred to herein as “Parameter-Free Interferent Rejection” (PFIR) methods. In one example embodiment using PFIR, for each of the possible interferent subsets ξ, the following calculations may be performed to compute an estimate of a calibration coefficient for each subset ξ. An average concentration may be estimated according to gₑₛₜ=Σg(ξ)p(ξ), where the sum is over the interferent subsets processed from the list of possible interferent subsets.

An example of an alternative embodiment of block 2130 includes the following steps and calculations.

Step 1: For a subset's N_{IF}, interferents, form a scaled interferent spectra matrix. In certain embodiments, the scaled interferent spectra matrix is the product of an interferent spectral matrix, IF, multiplied by an interferent concentration matrix, Tₘₐₓ, and can be written as: IF Tₘₐₓ. In certain such embodiments, the interferent concentration matrix Tₘₐₓ is a diagonal matrix having entries given by the maximum plasma concentrations for the various interferents.

Step 2: Calculate a covariance for the interferent component. If X denotes the IESD, the covariance of X, cov(X), is defined as the expectation E((X−mean(X))(X−mean(X))^{T}) and is

cov(X)≈XX^{T}/(N−1)−mean(X)mean(X)^{T}. (1)

As described above, the IESD (e.g., X) is obtained as a combination of Sample Population Spectra, C, with Combination Interferent Spectra (CIS): Xⱼ=Cⱼ+IFⱼξⱼ, therefore the covariance is:

cov(X)≈CC^{T}/(N−1)+IFΞΞ^{T}IF^{T}/(N−1)−mean(X)mean(X)^{T}, (2)

which can be written as,

cov(X)≈cov(C)+IF cov(Ξ)IF^{T}. (3)

If the weights in the weighting matrix Ξ are independent and identically distributed, the covariance of Ξ, cov(Ξ), is a diagonal matrix having along the diagonal the variance, v, of the samples in Ξ. The last equation may be written as

cov(X)≈V₀+vΦ, (4)

where V₀ is the covariance of the original sample population and Φ is the covariance of the IF spectral set.

Step 3: The group's covariance may be at least partially corrected for the presence of a single replicate of the Sample Population spectra with the IESD as formed from N_{IF} replicates of the Sample Population Spectra with Combined Interferent Spectra. This partial correction may be achieved by multiplying the second term in the covariance formula given above by a correction factor ρ:

V=V₀+ρvΦ, (5)

where ρ is a scalar weighting function that depends on the number of interferents in the group. In some embodiments, the scalar weighting function is ρ=N_{IF}/(N_{IF}+1). In certain embodiments, the variance v of the weights is assumed to be the variance of a log-normal random variable having a 95th percentile at a value of 1.0, and a standard deviation equal to half of the mean value.

Step 4: The eigenvectors and the corresponding eigenvalues of the covariance matrix V are determined using any suitable linear algebraic methods. The number of eigenvectors (and eigenvalues) is equal to the number of wavelengths L in the spectral measurements. The eigenvectors may be sorted based on decreasing order of their corresponding eigenvalues.

Step 5: The matrix of eigenvectors is decomposed so as to provide an orthogonal matrix Q. For example, in some embodiments, a QR-decomposition is performed, thereby yielding the matrix Q having orthonormal columns and rows.

Step 6: The following matrix operations are performed on the orthogonal matrix Q. For n=2 to L−1, the product P^{∥}ₙ=Q(:,1:n) Q(:,1:n)^{T} is calculated, where Q(:,1:n) denotes the submatrix comprising the first n columns of the full matrix Q. The orthogonal projection, P^{⊥}ₙ, away from the space spanned by Q(:,1:n) is determined by subtracting P^{∥}ₙ from the L×L identity matrix I. The n^{th} calibration vector is then determined from κₙ=P¹⁹⁵ₙα_{X}/α_{X}^{T}P^{⊥}ₙα_{X}, and the n^{th} error variance Eₙ is determined as the projection of the full covariance V onto the subspace spanned by κₙ as follows: Eₙ=κₙ^{T} Vκₙ.

The steps 4-6 of this example are an embodiment of the HLA technique.

In some embodiments, the calibration coefficient κ is selected as the calibration vector corresponding to the minimum error variance Eₙ. Thus, for example, the average group calibration coefficient κ may be found by searching among all the error variances for the error variance Eₙ that has the minimum value. The calibration coefficient is then selected as the n^{th} calibration vector κₙ corresponding to the minimum error variance Eₙ. In other embodiments, the calibration coefficient is determined by averaging some or all of the calibration vectors κₙ.

### Examples of Algorithm Results and Effects of Sample Population

Embodiments of the above-described methods have been used to estimate blood plasma glucose concentrations in humans. Four example experiments will now be described. The population of individuals from whom samples were obtained for analysis (estimation of glucose concentration) will be referred to as the “target population.” Infrared spectra obtained from the target population will be referred to as the “target spectra.” In the four example experiments, the target population included 41 intensive care unit (ICU) patients. Fifty-five samples were obtained from the target population.

### Example Experiment 1

In this example experiment, a partial least squares (PLS) regression method was applied to the infrared target spectra of the target patients' blood plasma to obtain the glucose estimates. In example experiment 1, estimated glucose concentration was not corrected for effects of interferents. The Sample Population used for the analysis included infrared spectra and independently measured glucose concentrations for 92 individuals selected from the general population. This Sample Population will be referred to as a “Normal Population.”

### Example Experiment 2

In example experiment 2, an embodiment of the Parameter-Free Interferent Rejection (PFIR) method was used to estimate glucose concentration for the same target population of patients in example experiment 1. The Sample Population was the Normal Population. In this example, calibration for Library Interferents was applied to the measured target spectra. The Library of Interferents included spectra of the 59 substances listed below:

Acetylsalicylic Acid
Ampicillin Sulbactam
Azithromycin
Aztreonam
Bacitracin
Benzyl Alcohol
Calcium Chloride
Calcium Gluconate
Cefazolin
Cefoparazone
Cefotaxime Sodium
Ceftazidime
Ceftriaxone
D_Sorbitol
Dextran
Ertapenem
Ethanol
Ethosuximide
Glycerol
Heparin
Hetastarch
Human Albumin
Hydroxy Butyric Acid
Imipenem Cilastatin
Iohexol
L_Arginine
Lactate Sodium
Magnesium Sulfate
Maltose
Mannitol
Meropenem
Oxylate Potassium
Phenytoin
Phosphates Potassium
Piperacillin
Piperacillin Tazobactam
PlasmaLyteA
Procaine HCl
Propylene Glycol
Pyrazinamide
Pyruvate Sodium
Pyruvic Acid
Salicylate Sodium
Sodium Acetate
Sodium Bicarbonate
Sodium Chloride
Sodium Citrate
Sodium Thiosulfate
Sulfadiazine
Urea
Uric Acid
Voriconazole
Xylitol
Xylose
PC 1 of Saline covariance
PC 2 of Saline covariance
PC 3 of Saline covariance
PC 4 of Saline covariance
ICU/Normal difference spectrum

In some embodiments, the calibration data set is determined according to two criteria: the calibration method itself (e.g., HLA, PLS, OLS, PFIR) and the intended application of the method. The calibration data set may comprise spectra and corresponding analyte levels derived from a set of plasma samples from the Sample Population. In some embodiments, e.g., those where an HLA calibration method is used, the calibration data set may also include spectra of the analyte of interest.

In the example experiments 1 and 2, the Sample Population was the Normal Population. Thus, samples were drawn from a population of normal individuals who did not have identifiable medical conditions that might affect the spectra of their plasma samples. For example, the sample plasma spectra typically did not show effects of high levels of medications or other substances (e.g., ethanol), or effects of chemicals that are indicative of kidney or liver malfunction.

In some embodiments, an analysis method may calibrate for deviations from the distribution defined by the calibration plasma spectra by identifying a “base” set of interferent spectra likely to be responsible for the deviation. The analysis method may then recalibrate with respect to an enhanced spectral data set. In some embodiments, the enhancement can be achieved by including the identified interferent spectra into the calibration plasma spectra. When it is anticipated that the target population may have been administered significant amounts of substances not present in the samples of the calibration set, or when the target population have many distinct interferents, estimation of the interferents present in the target spectrum may be subject to a large degree of uncertainty. In some cases, this may cause analyte estimation to be subject to errors.

Accordingly, in certain embodiments, the calibration data set may be enhanced beyond the base of “normal” samples to include a population of samples intended to be more representative of the target population. The enhancement of the calibration set may be generated, in some embodiments, by including samples from a sufficiently diverse range of individuals in order to represent the range of likely interferents (both in type and in concentration) and/or the normal variability in underlying plasma characteristics. The enhancement may, additionally or alternatively, be generated by synthesizing interferent spectra having a range of concentrations as described above (see, e.g., discussion of block 2310 in FIG. 23). Using the enhanced calibration set may reduce the error in estimating the analyte concentration in the target spectra.

### Example Experiments 3 and 4

Example experiments 3 and 4 use the analysis methods of example experiments 1 and 2, respectively (PLS without interferent correction and PFIR with interferent correction). However, example experiments 3 and 4 use a Sample Population having blood plasma spectral characteristics different from the Normal Population used in example experiments 1 and 2. In example experiments 3 and 4, the Sample Population was modified to include spectra of both the Normal Population and spectra of an additional population of 55 ICU patients. These spectra will be referred to as the “Normal+Target Spectra.” In experiments 3 and 4, the ICU patients included Surgical ICU patients, Medical ICU patients as well as victims of severe trauma, including a large proportion of patients who had suffered major blood loss. Major blood loss may necessitate replacement of the patient's total blood volume multiple times during a single day and subsequent treatment of the patient via electrolyte and/or fluid replacement therapies. Major blood loss may also require administration of plasma-expanding medications. Major blood loss may lead to significant deviations from the blood plasma spectra representative of a Normal Population. The population of 55 ICU patients (who provided the Target Spectra) has some similarities to the individuals for whom the analyses in experiments 1-4 were performed (e.g., all were ICU patients), but in these experiments, target spectra from individuals in the target population were not included in the Target Spectra.

Results of example experiments 1-4 are shown in the following table. The glucose concentrations estimated from the analysis method were compared to independently determined glucose measurements to provide an average prediction error and a standard deviation of the average prediction error. The table demonstrates that independent of the Sample Population used (e.g., either the Normal Population or the Normal+Target Population), calibrating for interferents reduces both the average prediction error and the standard deviation (e.g., compare the results for experiment 2 to the results for experiment 1 and compare the results for experiment 4 to the results for experiment 3). The table further demonstrates that independent of the analysis method used (e.g., either PLS or PFIR), using a Sample Population with more similarity to the target population (e.g., the Normal+Target Population) reduces both the average prediction error and the standard deviation (e.g., compare the results for experiment 3 to the results for experiment 1 and compare the results for experiment 4 to the results for experiment 2).

| Example | | | Average | Standard |
|---|---|---|---|---|
| Experiment | Interferent | Sample | Prediction Error | Deviation |
| No. | Calibration | Population | (mg/dL) | (mg/dL) |
| 1 | NO | Normal | 126 | 164 |
| 2 | YES | Normal | −6.8 | 23.2 |
| 3 | NO | Normal + | 8.2 | 16.9 |
| | | Target | | |
| 4 | YES | Normal + | 1.32 | 12.6 |
| | | Target | | |

Accordingly, embodiments of analysis methods that use a Sample Population that includes both normal spectra and spectra from individuals similar to those of the target population and that calibrate for possible interferents provide a good match between the estimated glucose concentration and the measured glucose concentration. As discussed above, a suitable Sample Population may be assembled from the Population Database in order to include normal spectra plus suitable target spectra from individuals that match a desired target population including, for example, ICU patients, trauma patients, a particular demographic group, a group having a common medical condition (e.g., diabetes), and so forth.

### User Interface

The system 400 can include a display system 414, for example, as depicted in FIG. 4. The display system 414 may comprise an input device including, for example, a keypad or a keyboard, a mouse, a touchscreen display, and/or any other suitable device for inputting commands and/or information. The display system 414 may also include an output device including, for example, an LCD monitor, a CRT monitor, a touchscreen display, a printer, and/or any other suitable device for outputting text, graphics, images, videos, etc. In some embodiments, a touchscreen display is advantageously used for both input and output.

The display system 414 can include a user interface 2400 by which users can conveniently and efficiently interact with the system 400. The user interface 2400 may be displayed on the output device of the system 400 (e.g., the touchscreen display). In some embodiments, the user interface 2400 is implemented and/or stored as one or more code modules, which may be embodied in hardware, firmware, and/or software.

FIGS. 24 and 25 schematically illustrate the visual appearance of embodiments of the user interface 2400. The user interface 2400 may show patient identification information 2402, which can include patient name and/or a patient ID number. The user interface 2400 also can include the current date and time 2404. An operating graphic 2406 shows the operating status of the system 400. For example, as shown in FIGS. 24 and 25, the operating status is “Running,” which indicates that the system 400 is fluidly connected to the patient (“Jill Doe”) and performing normal system functions such as infusing fluid and/or drawing blood. The user interface 2400 can include one or more analyte concentration graphics 2408, 2412, which may show the name of the analyte and its last measured concentration. For example, the graphic 2408 in FIG. 24 shows “Glucose” concentration of 150 mg/dL, while the graphic 2412 shows “Lactate” concentration of 0.5 mmol/L. The particular analytes displayed and their measurement units (e.g., mg/dL, mmol/L, or other suitable unit) may be selected by the user. The size of the graphics 2408, 2412 may be selected to be easily readable out to a distance such as, e.g., 30 feet. The user interface 2400 may also include a next-reading graphic 2410 that indicates the time until the next analyte measurement is to be taken. In FIG. 24, the time until next reading is 3 minutes, whereas in FIG. 25, the time is 6 minutes, 13 seconds.

The user interface 2400 can include an analyte concentration status graphic 2414 that indicates status of the patient's current analyte concentration compared with a reference standard. For example, the analyte may be glucose, and the reference standard may be a hospital ICU's tight glycemic control (TGC). In FIG. 24, the status graphic 2414 displays “High Glucose,” because the glucose concentration (150 mg/dL) exceeds the maximum value of the reference standard. In FIG. 25, the status graphic 2414 displays “Low Glucose,” because the current glucose concentration (79 mg/dL) is below the minimum reference standard. If the analyte concentration is within bounds of the reference standard, the status graphic 2414 may indicate normal (e.g., “Normal Glucose”), or it may not be displayed at all. The status graphic 2414 may have a background color (e.g., red, yellow, green, blue or some other color) when the analyte concentration exceeds the acceptable bounds of the reference standard.

In various implementations of the user interface 2400 a characteristic of the status graphic 2408 or 2412 displaying the concentration of the analyte can be varied (or otherwise emphasized) if the concentration of the analyte is predicted to be outside the acceptable bounds of the reference standard at a future time (e.g., less than or equal to about 5 minutes, less than or equal to about 10 minutes, less than or equal to about 15 minutes, less than or equal to about 20 minutes, less than or equal to about 30 minutes, less than or equal to about 45 minutes, less than or equal to about 60 minutes, less than or equal to about 90 minutes, less than or equal to about 120 minutes, etc.). The characteristic of the status graphic 2408 or 2412 can be a foreground and/or a background color. The characteristic of the status graphic 2408 or 2412 can be a visual state.

Various visual, auditory or other sensory approaches can be used to call attention to the display or a portion of the display. It can be advantageous to reduce anxiety of a patient while at the same time increasing awareness by a health-care provider. Such targeted but discrete emphasis can be accomplished, for example, by using visual cues that a health-care provider understands, but that do not add auditory alarms. Nevertheless, auditory alarms can be used for some particularly urgent circumstances. Visual emphasis effects can include foreground, background, or text colors, bright hues, increased color saturation or intensity, high contrast with surrounding visual colors, movement, animation, or dynamic displays, etc. Colors used can take advantage of existing or standardized cultural and/or medical meanings. For example, yellow can be a highly visual color that signifies caution but not an emergency. Red can signal something undesirable (or something that should be stopped), while green can signal something that is not dangerous or that is proceeding normally. These same colors can have other meanings in this or other contexts. Relative size or positioning of visual information can also be used for emphasis or de-emphasis. Larger symbols can call attention to more urgent or relevant information. In some embodiments, some colors (e.g., red and green) can be used for information about a current analyte value, while another color (e.g., yellow) can be used for information about a predicted or projected analyte value. Color, intensity, emphasis, etc. can change depending on the calculated certainty or uncertainty, and/or relative immediacy of a prediction, etc. These principles can apply to trend lines and other styles of data display (including tabular and bar graph displays, status graphics 2408 and 2412, etc. Three dimensional displays (with shading, contoured surfaces, multiple axes, movement into the screen to indicate forward movement in time, etc.) can be used and navigated using the user interface when multiple variables are being depicted.

For example, the status graphic 2408 or 2412 can have different foreground and/or background colors if the concentration of the analyte is predicted to be outside the acceptable bounds of the reference standard at a future time. For example, the status graphic 2408 or 2412 can have a yellow or an orange background color if the concentration of the analyte is predicted to be outside the acceptable bounds of the reference standard at a future time. The future time can be displayed, it can be adjustable, it can be modified through the display, and/or it can be set at a standard future time like 10 minutes, 30 minutes, 1 hour, 2 hours, etc. As another example, the status graphic 2408 or 2412 can have a green background or foreground color if the concentration of the analyte at the current measurement time and the predicted value for the concentration of the analyte at the future time is within the acceptable bounds of the reference standard. As yet another example, the status graphic 2408 or 2412 can be configured to blink and/or flash if the concentration of the analyte is predicted to be outside the acceptable bounds of the reference standard at a future time. Thus, for example, a nurse can be efficiently notified to change an insulin delivery rate based on measure glucose value and calculated or predicted rate of change.

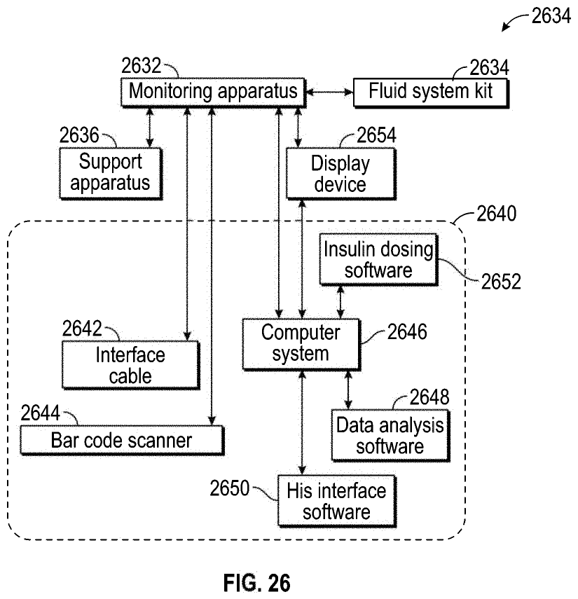

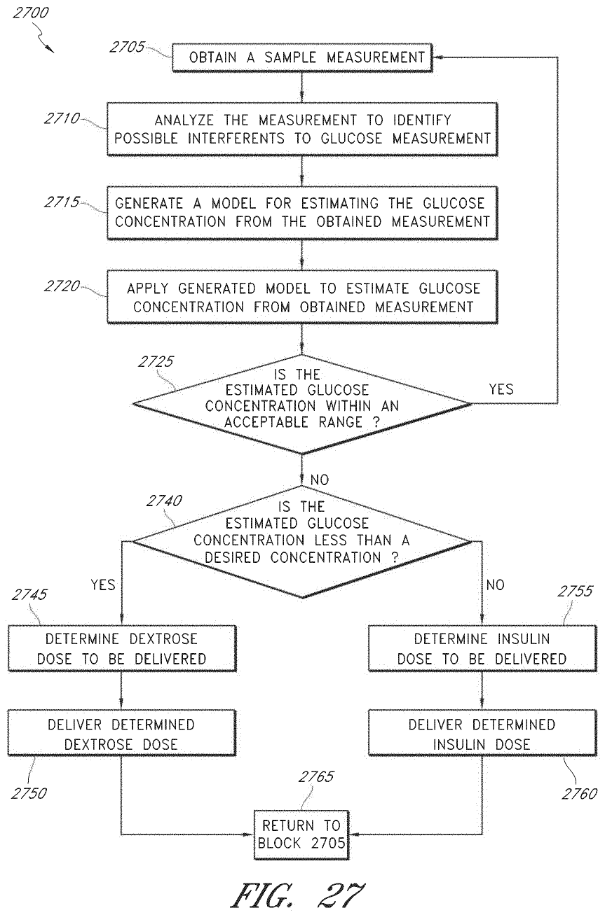

For instance, the concentration of the analyte measured at the current measurement time may be within the acceptable bounds of the reference standard. However, if the value of the concentration of the analyte predicted to be outside the acceptable bounds of the reference standard at a future time (e.g., less than or equal to about 5 minutes, less than or equal to about 10 minutes, less than or equal to about 15 minutes, less than or equal to about 20 minutes, less than or equal to about 30 minutes, less than or equal to about 45 minutes, less than or equal to about 60 minutes, less than or equal to about 90 minutes, less than or equal to about 120 minutes, etc.), then the status graphic 2408 or 2412 can be emphasized according to the principles described herein (e.g., have a yellow or an orange background color and/or be configured to blink and/or flash, etc.) to indicate to the health care provider that the concentration of the analyte may be dangerous in the future, or outside an acceptable range of a reference standard. Accordingly, a health care provider (e.g., a doctor, a nurse or a care giver) can be alerted to change the dose of an infusion substance (e.g., insulin or dextrose) based on a dosing protocol to avoid concentration of the analyte from being outside the acceptable bounds of the reference standard at the future time. In various implementations, a patient monitoring system (e.g., patient monitoring system 2630 of FIG. 26 and system 400 of FIG. 4) can be configured to provide a suggested dose for the infusion substance based on the predicted value of the concentration of the analyte as discussed below with reference to FIG. 27 and/or as described in various systems and methods described in U.S. application Ser. No. 12/249,831 (Atty. Docket No. OPTIS.203A) and Ser. No. 12/559,328 (Atty. Docket No. OPTIS.247A), the entire disclosures of which are incorporated herein by reference, for all purposes.

The predicted value of the concentration of the analyte at a future time can be calculated using instructions stored in a processor (e.g., algorithm processor 416 of system 400 or the computer system 2646 of the system 2630) of a patient monitoring system (e.g., patient monitoring system 2630 of FIG. 26 and system 400 of FIG. 4).

The predicted value of the concentration of the analyte at a future time can be determined from one or more previous measurements of the analyte concentration. For example, any one of the various systems and methods of determining the predicted value of the concentration of the analyte at a future time are described in U.S. application Ser. No. 12/249,831 (Atty. Docket No. OPTIS.203A) and Ser. No. 12/559,328 (Atty. Docket No. OPTIS.247A)—the entire disclosures of which are incorporated herein by reference, for all purposes—can be employed to determine predicted value of the concentration of the analyte at a future time.

As another example, the predicted value of the concentration of the analyte at a future time can be determined from a rate of change in the concentration of the analyte calculated based on one or more previous measurements of the analyte concentration. For instance, the average rate of change in the concentration of the analyte over a past certain period of time (e.g., about 10 minutes, about 20 minutes, about 30 minutes, about 45 minutes, about 1 hour, etc.) can be determined and the value of the concentration of the analyte in the next certain period of time (e.g., about 10 minutes, about 20 minutes, about 30 minutes, about 45 minutes, about 1 hour, etc.) can be calculated assuming a predicted rate of change of concentration that is proportional to the determined average rate of change in the concentration of the analyte. In various implementations, the predicted rate of change of concentration can depend on the average rate of change in the concentration of the analyte over a past certain period of time and a dose of the infusion substance (e.g., insulin or dextrose) in that period of time.

In various implementations, the predicted rate of change of concentration can be depend on a first average rate of change in the concentration of the analyte over a first time interval previous to the current measurement time, a second average rate of change in the concentration of the analyte over a second time interval previous to the current measurement time, a third average rate of change in the concentration of the analyte over a third time interval previous to the current measurement time and so on. Additionally, the predicted rate of change of concentration can be depend on a first dose of the infusion substance (e.g., insulin or dextrose) in the first time interval, a second dose of the infusion substance (e.g., insulin or dextrose) in the second time interval, a third dose of the infusion substance (e.g., insulin or dextrose) in the second time interval and so on.

As another example, the predicted value of the concentration of the analyte at a future time can be determined from a trend in the values of the concentration of the analyte obtained in one or more previous measurements of the analyte concentration. For instance, the predicted value of the concentration of the analyte at a future time can be determined from a slope of the curve of at least two values of the concentration of the analyte obtained at a time previous to the current measurement time.

In various implementations, the patient monitoring system can be configured to determine the concentration of the analyte at time t1 and rate of change of concentration of the analyte from a previous measurement time t0 and calculate the predicted value of the concentration at a future time tn. The patient monitoring system can be configured to subsequently determine the concentration of the analyte at time t2 and rate of change of concentration of the analyte from the previous measurement time t1 and refine the predicted value of the concentration at the future time tn. In this manner the predicted value of the concentration at the future time tn can be calculated and refined on a moving window basis based on the rate of change in the concentration of the analyte at the current measurement from a previous measurement time. Such a moving window can advantageously “move” in sync with the advance of actual time by having an initial window opening and final window closing time, each of which is prescribed to be a fixed amount of time into the future. The window's parameters (e.g., the width of the window, its distance into the future from the present time, statistical error associated with the data it displays, etc.) can be adjusted by a user, or they can be set to avoid confusion.

Various embodiments and aspects of this disclosure can be combined, in particular those referring to a graphic user interface. Accordingly, the disclosure herein relating to visual emphasis of predicted future values can be combined with the disclosure relating to adding annotations to a computer memory using a touch-screen with bookmarks and visual flags indicating when a note has been entered by a user. Thus, a flashing yellow indicator can be annotated (and the time stamp of the annotation, the identity of the source of the note, etc. can be recorded) when a doctor or nurse wishes to record that the alert was noticed but ignored or overridden because of a factor that does not play a part in the automatic machine algorithm that led to the alert. Other synergies existing between various aspects of the disclosure herein relating to user interfaces, displays and interactions therewith, and the ability of a system to provide accurate analyte information and projections.

The user interface 2400 can include one or more trend indicators 2416 that provide a graphic indicating the time history of the concentration of an analyte of interest. In FIGS. 24 and 25, the trend indicator 2416 comprises a graph of the glucose concentration (in mg/dL) versus elapsed time (in hours) since the measurements started. The graph includes a trend line 2418 indicating the time-dependent glucose concentration. In other embodiments, the trend line 2418 can include measurement error bars and may be displayed as a series of individual data points. In FIG. 25, the glucose trend indicator 2416 is shown as well as a trend indicator 2430 and trend line 2432 for the lactate concentration. In some embodiments, a user may select whether none, one, or both trend indicators 2416, 2418 are displayed. In some embodiments, one or both of the trend indicators 2416, 2418 may appear only when the corresponding analyte is in a range of interest such as, for example, above or below the bounds of a reference standard. A graphic user interface can also indicate predicted values using a trend display. For example, a dotted line can be displayed that projects into a portion of the graph depicting a future time. A separate value can be displayed for a given future time (although in many cases, this value may be less prominently displayed than the actual measured value). The trend line (or a portion thereof) can be displayed in various colors to indicate different levels of predicted future values. For example, a tip of the trend line can be displayed as yellow if it is predicted to fall within a dangerous range within the next hour.

The user interface 2400 can include one or more buttons 2420-2426 that can be actuated by a user to provide additional functionality or to bring up suitable context-sensitive menus and/or screens. For example, in the embodiments shown in FIG. 24 and FIG. 25, four buttons 2420-2426 are shown, although fewer or more buttons are used in other embodiments. The button 2420 (“End Monitoring”) may be pressed when one or more removable portions (see, e.g., 710 of FIG. 7) are to be removed. In many embodiments, because the removable portions 710,712 are not reusable, a confirmation window appears when the button 2420 is pressed. If the user is certain that monitoring should stop, the user can confirm this by actuating an affirmative button in the confirmation window. If the button 2420 were pushed by mistake, the user can select a negative button in the confirmation window. If “End Monitoring” is confirmed, the system 400 performs appropriate actions to cease fluid infusion and blood draw and to permit ejection of a removable portion (e.g., the removable portion 710).

The button 2422 (“Pause”) may be actuated by the user if patient monitoring is to be interrupted but is not intended to end. For example, the “Pause” button 2422 may be actuated if the patient is to be temporarily disconnected from the system 400 (e.g., by disconnecting the tubes 306). After the patient is reconnected, the button 2422 may be pressed again to resume monitoring. In some embodiments, after the “Pause” button 2422 has been pressed, the button 2422 displays “Resume.”

The button 2424 (“Delay 5 Minutes”) causes the system 400 to delay the next measurement by a delay time period (e.g., 5 minutes in the depicted embodiments). Actuating the delay button 2424 may be advantageous if taking a reading would be temporarily inconvenient, for example, because a health care professional is attending to other needs of the patient. The delay button 2424 may be pressed repeatedly to provide longer delays. In some embodiments, pressing the delay button 2424 is ineffective if the accumulated delay exceeds a maximum threshold. The next-reading graphic 2410 automatically increases the displayed time until the next reading for every actuation of the delay button 2424 (up to the maximum delay).

The button 2426 (“Dose History”) may be actuated to bring up a dosing history window that displays patient dosing history for an analyte or medicament of interest. For example, in some embodiments, the dosing history window displays insulin dosing history of the patient and/or appropriate hospital dosing protocols. A nurse attending the patient can actuate the dosing history button 2426 to determine the time when the patient last received an insulin dose, the last dosage amount, and/or the time and amount of the next dosage. The system 400 may receive the patient dosing history via wired or wireless communications from a hospital information system.

In other embodiments, the user interface 2400 can include additional and/or different buttons, menus, screens, graphics, etc. that are used to implement additional and/or different functionalities.

Related Components

FIG. 26 schematically depicts various components and/or aspects of a patient monitoring system 2630 and how those components and/or aspects relate to each other. In some embodiments, the monitoring system 2630 can be the apparatus 100 for withdrawing and analyzing fluid samples. Some of the depicted components can be included in a kit containing a plurality of components. Some of the depicted components, including, for example, the components represented within the dashed rounded rectangle 2640 of FIG. 26, are optional and/or can be sold separately from other components.

The patient monitoring system 2630 shown in FIG. 26 includes a monitoring apparatus 2632. The monitoring apparatus 2632 can be the monitoring device 102, shown in FIG. 1 and/or the system 400 of FIG. 4. The monitoring apparatus 2632 can provide monitoring of physiological parameters of a patient. In some embodiments, the monitoring apparatus 2632 measures glucose and/or lactate concentrations in the patient's blood. In some embodiments, the measurement of such physiological parameters is substantially continuous. The monitoring apparatus 2632 may also measure other physiological parameters of the patient. In some embodiments, the monitoring apparatus 2632 is used in an intensive care unit (ICU) environment. In some embodiments, one monitoring apparatus 2632 is allocated to each patient room in an ICU. The patient monitoring system 2630 can comprise a display device 2654 (e.g., display system 414 of FIG. 4) to display the measured physiological parameters of the patient. The display device 2654 of the patient monitoring system 2630 can include a user interface (e.g., user interface 2400) to allow a health care professional (e.g., doctor, nurse, a caregiver) to interact conveniently and efficiently interact with the display device 2654.

The patient monitoring system 2630 can include an optional interface cable 2642. In some embodiments, the interface cable 2642 connects the monitoring apparatus 2632 to a patient monitor (not shown). The interface cable 2642 can be used to transfer data from the monitoring apparatus 2632 to the patient monitor for display. In some embodiments, the patient monitor is a bedside cardiac monitor having a display that is located in the patient room (see, e.g., the user interface 2400 shown in FIG. 24 and FIG. 25.) In some embodiments, the interface cable 2642 transfers data from the monitoring apparatus 2632 to a central station monitor and/or to a hospital information system (HIS). The ability to transfer data to a central station monitor and/or to a HIS may depend on the capabilities of the patient monitor system.

In the embodiment shown in FIG. 26, an optional bar code scanner 2644 is connected to the monitoring apparatus 2632. In some embodiments, the bar code scanner 2644 is used to enter patient identification codes, nurse identification codes, and/or other identifiers into the monitoring apparatus 2632. In some embodiments, the bar code scanner 2644 contains no moving parts. The bar code scanner 2644 can be operated by manually sweeping the scanner 2644 across a printed bar code or by any other suitable means. In some embodiments, the bar code scanner 2644 includes an elongated housing in the shape of a wand.

The patient monitoring system 2630 includes a fluid system kit 2634 connected to the monitoring apparatus 2632. In some embodiments, the fluid system kit 2634 includes fluidic tubes that connect a fluid source to an analytic subsystem. For example, the fluidic tubes can facilitate fluid communication between a blood source or a saline source and an assembly including a sample holder and/or a centrifuge. In some embodiments, the fluid system kit 2634 includes many of the components that enable operation of the monitoring apparatus 2632. In some embodiments, the fluid system kit 2634 can be used with anti-clotting agents (such as heparin), saline, a saline infusion set, a patient catheter, a port sharing IV infusion pump, and/or an infusion set for an IV infusion pump, any or all of which may be made by a variety of manufacturers. In some embodiments, the fluid system kit 2634 includes a monolithic housing that is sterile and disposable. In some embodiments, at least a portion of the fluid system kit 2634 is designed for single patient use. For example, the fluid system kit 2634 can be constructed such that it can be economically discarded and replaced with a new fluid system kit 2634 for every new patient to which the patient monitoring system 2630 is connected. In addition, at least a portion of the fluid system kit 2634 can be designed to be discarded after a certain period of use, such as a day, several days, several hours, three days, a combination of hours and days such as, for example, three days and two hours, or some other period of time. Limiting the period of use of the fluid system kit 2634 may decrease the risk of malfunction, infection, or other conditions that can result from use of a medical apparatus for an extended period of time.

In some embodiments, the fluid system kit 2634 includes a connector with a luer fitting for connection to a saline source. The connector may be, for example, a three-inch pigtail connector. In some embodiments, the fluid system kit 2634 can be used with a variety of spikes and/or IV sets used to connect to a saline bag. In some embodiments, the fluid system kit 2634 also includes a three-inch pigtail connector with a luer fitting for connection to one or more IV pumps. In some embodiments, the fluid system kit 2634 can be used with one or more IV sets made by a variety of manufacturers, including IV sets obtained by a user of the fluid system kit 2634 for use with an infusion pump. In some embodiments, the fluid system kit 2634 includes a tube with a low dead volume luer connector for attachment to a patient vascular access point. For example, the tube can be approximately seven feet in length and can be configured to connect to a proximal port of a cardiovascular catheter. In some embodiments, the fluid system kit 2634 can be used with a variety of cardiovascular catheters, which can be supplied, for example, by a user of the fluid system kit 2634.

As shown in FIG. 26, the monitoring apparatus 2632 is connected to a support apparatus 2636, such as an IV pole. The support apparatus 2636 can be customized for use with the monitoring apparatus 2632. A vendor of the monitoring apparatus 2632 may choose to bundle the monitoring apparatus 2632 with a custom support apparatus 2636. In some embodiments, the support apparatus 2636 includes a mounting platform for the monitoring apparatus 2632. The mounting platform can include mounts that are adapted to engage threaded inserts in the monitoring apparatus 2632. The support apparatus 2636 can also include one or more cylindrical sections having a diameter of a standard IV pole, for example, so that other medical devices, such as IV pumps, can be mounted to the support apparatus. The support apparatus 2636 can also include a clamp adapted to secure the apparatus to a hospital bed, an ICU bed, or another variety of patient conveyance device.

In the embodiment shown in FIG. 26, the monitoring apparatus 2632 is electrically connected to an optional computer system 2646. The computer system 2646 can comprise one or multiple computers, and it can be used to communicate with one or more monitoring devices. In an ICU environment, the computer system 2646 can be connected to at least some of the monitoring devices in the ICU. The computer system 2646 can be used to control configurations and settings for multiple monitoring devices (for example, the system can be used to keep configurations and settings of a group of monitoring devices common). The computer system 2646 can also run optional software, such as data analysis software 2648, HIS interface software 2650, and insulin dosing software 2652.

In some embodiments, the computer system 2646 runs optional data analysis software 2648 that organizes and presents information obtained from one or more monitoring devices. In some embodiments, the data analysis software 2648 collects and analyzes data from the monitoring devices in an ICU. The data analysis software 2648 can also present charts, graphs, and statistics to a user of the computer system 2646.

In some embodiments, the computer system 2646 runs optional hospital information system (HIS) interface software 2650 that provides an interface point between one or more monitoring devices and an HIS. The HIS interface software 2650 may also be capable of communicating data between one or more monitoring devices and a laboratory information system (LIS).

In some embodiments, the computer system 2646 runs optional insulin dosing software 2652 that provides a platform for implementation of an insulin dosing regimen. In some embodiments, the hospital tight glycemic control protocol is included in the software. The protocol allows computation of proper insulin doses for a patient connected to a monitoring device 2646. The insulin dosing software 2652 can communicate with the monitoring device 2646 to ensure that proper insulin doses are calculated.

### Analyte Control and Monitoring

In some embodiments, it may be advantageous to control a level of an analyte (e.g., glucose) in a patient using an embodiment of an analyte detection system described herein. Although certain examples of glucose control are described below, embodiments of the systems and methods disclosed herein may be used to monitor and/or control other analytes (e.g., lactate).

For example, diabetic individuals control their glucose levels by administration of insulin. If a diabetic patient is admitted to a hospital or ICU, the patient may be in a condition in which he or she cannot self-administer insulin. Advantageously, embodiments of the analyte detection systems disclosed herein may be used to control the level of glucose in the patient. Additionally, it has been found that a majority of patients admitted to the ICU exhibit hyperglycemia without having diabetes. In such patients it may be beneficial to monitor and control their blood glucose level to be within a particular range of values. Further, it has been shown that tightly controlling blood glucose levels to be within a stringent range may be beneficial to patients undergoing surgical procedures.

A patient admitted to the ICU or undergoing surgery may be administered a variety of drugs and fluids such as Hetastarch, intravenous antibiotics, intravenous glucose, intravenous insulin, intravenous fluids such as saline, etc., which may act as interferents and make it difficult to determine the blood glucose level. Moreover, the presence of additional drugs and fluids in the blood stream may require different methods for measuring and controlling blood glucose level. Also, the patient may exhibit significant changes in hematocrit levels due to blood loss or internal hemorrhage, and there can be unexpected changes in the blood gas level or a rise in the level of bilirubin and ammonia levels in the event of an organ failure. Embodiments of the systems and methods disclosed herein advantageously may be used to monitor and control blood glucose (and/or other analytes) in the presence of possible interferents to estimation of glucose and for patients experiencing health problems.

In some environments, Tight Glycemic Control (TGC) can include: (1) substantially continuous monitoring (which can include periodic monitoring, at relatively frequent intervals of every 1, 5, 15, 30, 45, and/or 60 minutes, for example) of glucose levels; (2) determination of substances that tend to increase glucose levels (e.g., sugars such as dextrose) and/or decrease glucose levels (e.g., insulin); and/or (3) responsive delivery of one or more of such substances, if appropriate under the controlling TGC protocol. For example, one possible TGC protocol can be achieved by controlling glucose within a relatively narrow range (for example between 70 mg/dL to 110 mg/dL). As will be further described, in some embodiments, TGC may be achieved by using an analyte monitoring system to make continuous and/or periodic but frequent measurements of glucose levels.

In some embodiments, the analyte detection system schematically illustrated in FIGS. 4, 5, and 6 may be used to regulate the concentration of one or more analytes in the sample in addition to determining and monitoring the concentration of the one or more analytes. In some cases, the analyte detection system may be used in an ICU to monitor (and/or control) analytes that may be present in patients experiencing trauma. In some implementations, the concentration of the analytes is regulated to be within a certain range. The range may be predetermined (e.g., according to a hospital protocol or a physician's recommendation), or the range may be adjusted as conditions change.

In an example of glycemic control, a system can be used to determine and monitor the concentration of glucose in the sample. If the concentration of glucose falls below a lower threshold, glucose from an external source can be supplied. If the concentration of glucose increases above an upper threshold, insulin from an external source can be supplied. In some embodiments, glucose or insulin may be infused in a patient continuously over a certain time interval or may be injected in a large quantity at once (referred to as “bolus injection”).

In some embodiments, a glycemic control system may be capable of delivering glucose, dextrose, glycogen, and/or glucagon from an external source relatively quickly in the event of hypoglycemia. As discussed, embodiments of the glycemic control system may be capable of delivering insulin from an external source relatively quickly in the event of hyperglycemia.

Returning to FIGS. 5 and 6, these figures schematically illustrate embodiments of a fluid handling system that comprise optional analyte control subsystems 2780. The analyte control subsystem 2780 may be used for providing control of an analyte such as, e.g., glucose, and may provide delivery of the analyte and/or related substances (e.g., dextrose solution and/or insulin in the case of glucose). The analyte control subsystem 2780 comprises a source 2782 such as, for example, the analyte (or a suitable compound related to the analyte) dissolved in water or saline. For example, if the analyte is glucose, the source 2782 may comprise a bag of dextrose solution (e.g., Dextrose or Dextrose 50%). The source 2782 can be coupled to an infusion pump (not shown). The source 2782 and the infusion pump can be provided separately from the analyte control subsystem 2780. For example, a hospital advantageously can use existing dextrose bags and infusion pumps with the subsystem 2780.

As schematically illustrated in FIGS. 5 and 6, the source 2782 is in fluid communication with the patient tube 512 via a tube 2784 and suitable connectors. A pinch valve 2786 may be disposed adjacent the tube 2784 to regulate the flow of fluid from the source 2782. A patient injection port can be located at a short distance from the proximal port of the central venous catheter or some other catheter connected to the patient.

In an example implementation for glycemic control, if the analyte detection system determines that the level of glucose has fallen below a lower threshold value (e.g., the patient is hypoglycemic), a control system (e.g., the fluid system controller 405 in some embodiments) controlling an infusion delivery system may close the pinch valves 521 and/or 542 to prevent infusion of insulin and/or saline into the patient. The control system may open the pinch valve 2786 and dextrose solution from the source 2782 can be infused (or alternatively injected as a bolus) into the patient. After a suitable amount of dextrose solution has been infused to the patient, the pinch valve 2786 can be closed, and the pinch valves 521 and/or 542 can be opened to allow flow of insulin and/or saline. In some systems, the amount of dextrose solution for infusion (or bolus injection) may be calculated based on one or more detected concentration levels of glucose. The source 2782 advantageously may be located at a short enough fluidic distance from the patient such that dextrose can be delivered to the patient within a time period of about one to about ten minutes. In other embodiments, the source 2782 can be located at the site where the patient tube 512 interfaces with the patient so that dextrose can be delivered within about one minute.

If the analyte detection system determines that the level of glucose has increased above an upper threshold value (e.g., the patient is hyperglycemic), the control system may close the pinch valves 542 and/or 2786 to prevent infusion of saline and/or dextrose into the patient. The control system may open the pinch valve 521, and insulin can be infused (or alternatively injected as a bolus) into the patient. After a suitable amount of insulin has been infused (or bolus injected) to the patient, the control system can close the pinch valve 521 and open the pinch valves 542 and/or 2786 to allow flow of saline and/or glucose. The suitable amount of insulin may be calculated based on one or more detected concentration levels of glucose in the patient. The insulin source 518 advantageously may be located at a short enough fluidic distance from the patient such that insulin can be delivered to the patient within about one to about ten minutes. In other embodiments, the insulin source 518 may be located at the site where the patient tube 512 interfaces with the patient so that insulin can be delivered to the patient within about one minute.

In some embodiments, sampling bodily fluid from a patient and providing medication to the patient may be achieved through the same lines of the fluid handling system. For example, in some embodiments, a port to a patient can be shared by alternately drawing samples and medicating through the same line. In some embodiments, a bolus can be provided to the patient at regular intervals (in the same or different lines). For example, a bolus of insulin can be provided to a patient after meals. In another embodiment comprising a shared line, a bolus of medication can be delivered when returning part of a body fluid sample back to the patient. In some implementations, the bolus of medication is delivered midway between samples (e.g., every 7.5 minutes if samples are drawn every 15 minutes). In other embodiment, a dual lumen tube can be used, wherein one lumen is used for the sample and the other lumen to medicate. In yet another embodiment, an analyte detection system (e.g., an “OptiScanner®” monitor) may provide suitable commands to a separate insulin pump (on a shared port or different line).

Example Method for Glycemic Control

FIG. 27 is a flowchart that schematically illustrates an example embodiment of a method 2700 of providing analyte control. The example embodiment is directed toward one possible implementation for glycemic control (including but not limited to tight glycemic control) and is intended to illustrate certain aspects of the method 2700 and is not intended to limit the scope of possible analyte control methods. In block 2705, a glucose monitoring apparatus (e.g., the monitoring apparatus 2632 of FIG. 26) draws a sample (e.g., a blood or blood plasma sample) from a sample source (e.g., a patient) and obtains a measurement from the sample (e.g., a portion of the drawn sample). The measurement may comprise an optical measurement such as, for example, an infrared spectrum of the sample. In block 2710, the measurement sample is analyzed to identify possible interferents to an estimation of the glucose concentration in the measurement sample. In block 2715, a model is generated for estimating the glucose concentration from the obtained measurement. In some embodiments, models developed from the algorithms describe above with reference to FIGS. 21-23 are used. The generated model may reduce or minimize effects of the identified interferents on the estimated glucose concentration, in certain embodiments. In block 2720, an estimated glucose concentration is determined from the model and the obtained measurement. In block 2725, the estimated glucose concentration in the sample is compared to an acceptable range of concentrations. The acceptable range may be determined according to a suitable glycemic control protocol such as, for example, a TGC protocol. For example, in certain TGC protocols the acceptable range may be a glucose concentration in a range from about 70 mg/dL to about 110 mg/dL. If the estimated glucose concentration lies within the acceptable range, the method 2700 returns to block 2705 to obtain the next sample measurement, which may be made within about one to about thirty minutes (e.g., every fifteen minutes).

In block 2725, if the estimated glucose concentration is outside the acceptable range of concentrations, then the method 2700 proceeds to block 2740 in which the estimated glucose concentration is compared with a desired glucose concentration. The desired glucose concentration may be based on, for example, the acceptable range of glucose concentrations, the parameters of the particular glycemic protocol, the patient's estimated glucose concentration, and so forth. If the estimated glucose concentration is below the desired concentration (e.g., the patient is hypoglycemic), a dose of dextrose to be delivered to the patient is calculated in block 2745. This calculation may take into account various factors including, for example, one or more estimated glucose concentrations, presence of additional drugs in the patient's system, time taken for dextrose to be assimilated by the patient, and the delivery method (e.g., continuous infusion or bolus injection). In block 2750, a fluid delivery system (e.g., a system such as the optional subsystem 2780 shown in FIGS. 5 and 6) delivers the calculated dose of dextrose to the patient.

In block 2740, if the estimated glucose concentration is greater than the desired concentration (e.g., the patient is hyperglycemic), a dose of insulin to be delivered is calculated in block 2755. The dose of insulin may depend on various factors including, for example, one or more estimated glucose concentrations in the patient, presence of other drugs, type of insulin used, time taken for insulin to be assimilated by the patient, method of delivery (e.g., continuous infusion or bolus injection), etc. In block 2750, a fluid delivery system (e.g., the optional subsystem 2780 shown in FIGS. 5 and 6) delivers the calculated dose of insulin to the patient.

In block 2765, the method 2700 returns to block 2705 to await the start of the next measurement cycle, which may be within about one to about thirty minutes (e.g., every fifteen minutes). In some embodiments, the next measurement cycle begins at a different time than normally scheduled in cases in which the estimated glucose concentration lies outside the acceptable range of concentrations under the glycemic protocol. Such embodiments advantageously allow the system to monitor response of the patient to the delivered dose of dextrose (or insulin). In some such embodiments, the time between measurement cycles is reduced so the system can more accurately monitor analyte levels in the patient.

Examples of Some Possible Additional or Alternative Analytes

Although examples of control and/or monitoring has been described in the illustrative context of glycemic control, embodiments of the systems and methods can be configured for control and/or monitoring of one or more of many possible analytes, in addition to or instead of glucose. Monitor and/or control of analytes may be particularly helpful in ICUs, which receive patients experiencing trauma. For example, another parameter that can be monitored is level of Hemoglobin (Hb). If the Hb level of a patient goes down without an apparent external reason, the patient could be suffering from internal bleeding. Indeed, many ICU patients (some estimate as many as 10%) suffer from what appears to be spontaneous internal bleeding that may not be otherwise detectable until the consequences are too drastic to easily overcome. In some embodiments, level of Hb can be measured indirectly, because its relationship to oxygen in the veins and arteries (at different points in the vasculature with respect to the heart and lungs) is understood. In some embodiments, the apparatus, systems and methods described herein can be useful for measuring a level of Hb.

Another parameter that can be monitored is lactate level, which can be related to sepsis or toxic shock. Indeed, high levels and/or rapid rise in lactate levels can be correlated to organ failure and oxygenation problems in the blood and organs. However, other direct measures of the biological effects related to lactate level problems can be difficult to measure, for example, only becoming measurable with a delay (e.g., 2-6 hours later). Thus, measurement of lactate level can help provide a valuable early warning of other medical problems. Indeed, if a problem with lactate levels is detected, a nurse or doctor may be able to prevent the correlated problems by providing more fluids.

Another parameter that can be monitored is central venous oxygen saturation (ScvO2). It can be advantageous to try to maintain an ScvO2 of 65-70% or greater in ICU patients (to help avoid sepsis, for example). In some embodiments, the apparatus, systems, and methods described herein can be useful for measuring a level of ScvO2.

Levels of lactate and ScvO2 in a patient can be used together to provide information and/or warnings to a health care provider, which can be especially useful in an ICU setting. For example, if lactate and ScvO2 are both high, a warning can be provided (e.g., automatically using an alarm). If lactate is high, but ScvO2 is low, a patient may benefit from additional fluids. If ScvO2 is high, but lactate is low, a cardiac problem may be indicated. Thus, a system that provides information about both lactate and ScvO2 can be very beneficial to a patient, especially, for example, in the ICU environment. Although lactate and ScvO2 have been used as an illustrative example, in other embodiments different combinations of analytes may be monitored and used to provide information and/or warnings to a health care provider.

### Annotation of Data

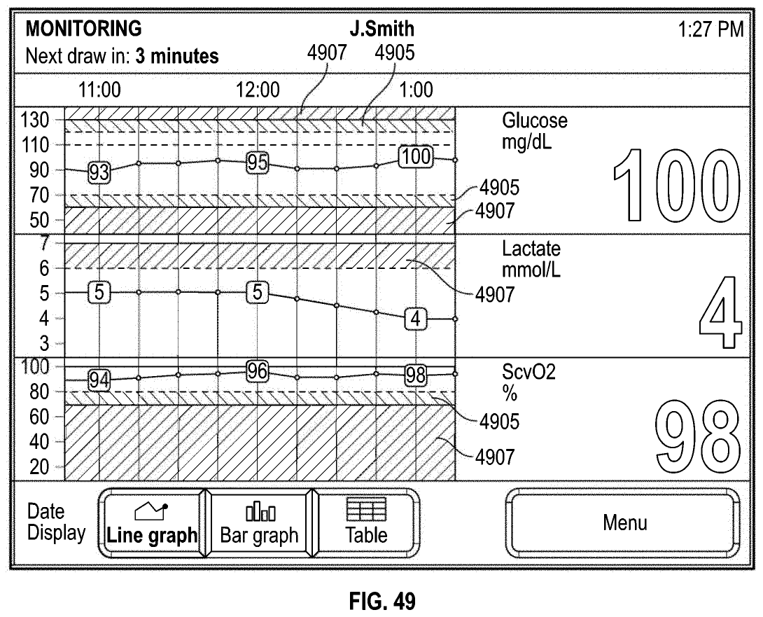

Data obtained or created by the described systems can be conveyed to users (e.g., doctors, nurses, patients, researchers, technicians, etc.) visually. For example, various aspects of a user interface are described above under the heading “USER INTERFACE.” As noted above, such an interface can comprise a touchscreen or other interactive display or surface. Examples of graphical user interfaces (GUIs) are provided in FIG. 24 and FIG. 25, for example. Additional examples of GUIs are provided in the following U.S. patent Publication Nos.: US 2008/0077073, FIGS. 49-59C, paragraphs [0461]-[0505]; US 2008/0077072, FIGS. 49-59C, paragraphs [0475]-[0519]; US 2009/0036764, FIGS. 24-25, 48-59 and the related description; US 2009/0160656, FIGS. 24-25, 30, and the related description; and U.S. Provisional Patent Application 60/953,454 (to which U.S. patent application Ser. No. 11/839,487 claims priority).

One useful way to display historical readings of analyte measurements is to plot such readings graphically, with older readings to the left and newer readings to the right, and with higher readings toward the top of the screen, and lower readings toward the bottom. Values for a given reading can be plotted on a Cartesian grid, which continually or periodically scrolls so that only the most recent readings are typically visible on the default view. Points representing measurements can be plotted and connected with lines (e.g., straight, or otherwise interpolated) to make it easier to visualize a general pattern or direction. An area above or below the lines can be a different color than an area on the other side of the line. For example, the display can be generally black or blank above the line, but colored below the line. If a reading is within a range predicted or desired by the physician or by the system or by the user, the line or the area under the line can be green, for example. If a reading does not meet those (or other relevant) criteria, a red color can be used.

Many of the above-described examples and figures provide for a serial plot of periodic, regular data movements. However a user may desire to annotate this data (e.g., using a touch-screen interface, stylus, mouse, etc.) For example, a doctor may desire to provide a graphical reminder that a change in dose occurred at a time corresponding to a data point plotted on the display. In some embodiments, a the doctor may touch the screen at that point, which can cause the screen to display a keyboard or other interface for accepting input such as text, numbers, a photo, a voice note, etc. That input can be associated with the time and data touched or otherwise indicated by the doctor. An indication (e.g., a visual cue such as a highlighted portion, a bubble (e.g., unexpanded until touched), etc. can be provided when the data is subsequently viewed, showing or indicating availability of the information previously inserted, stored, and associated with that data.

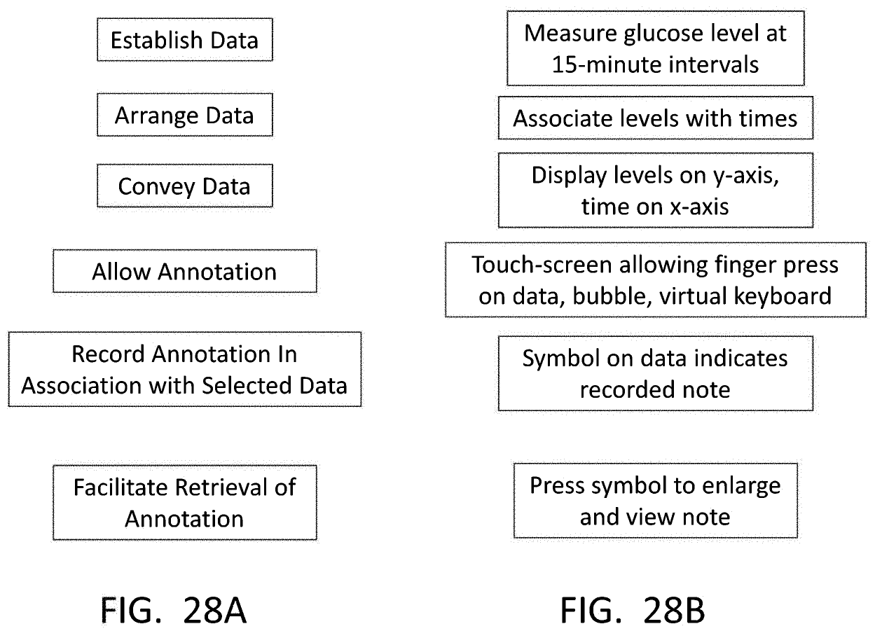

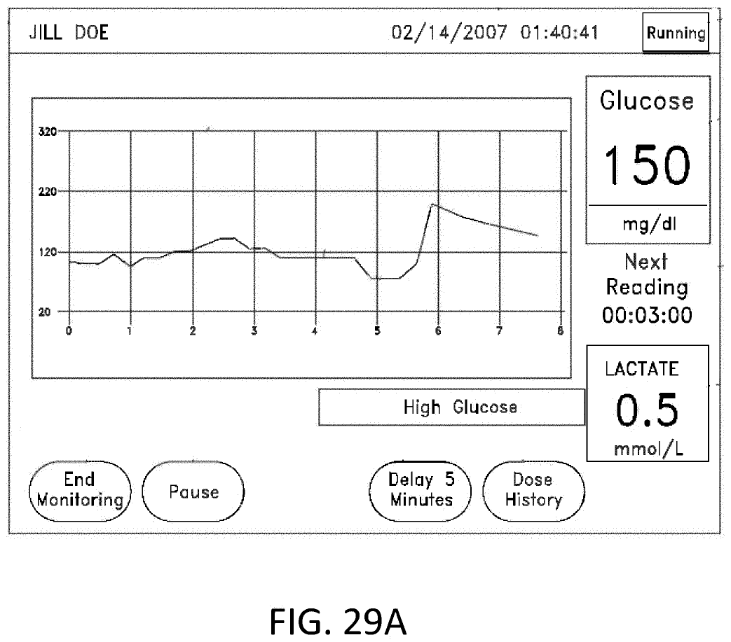

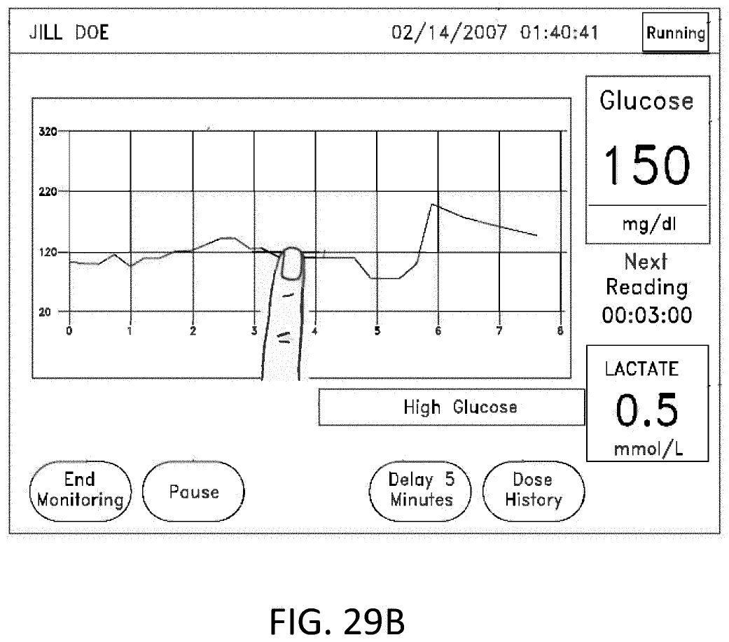

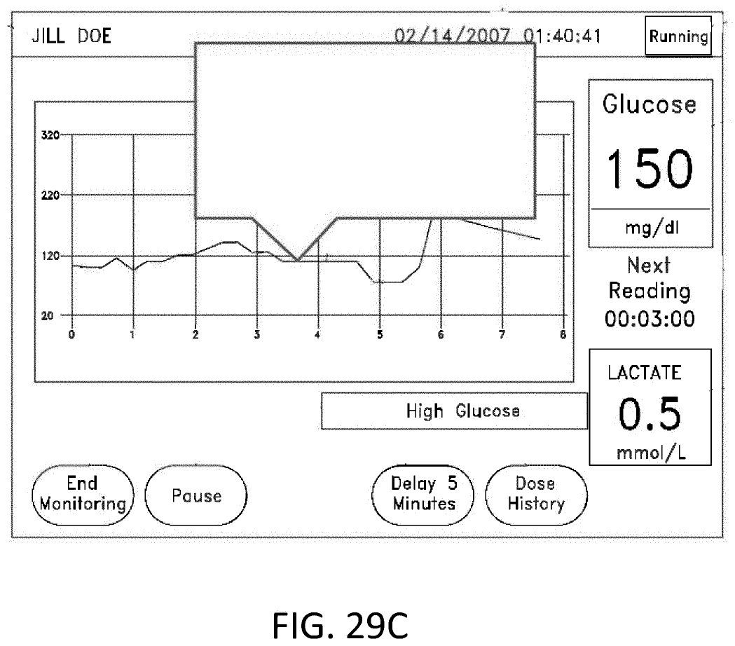

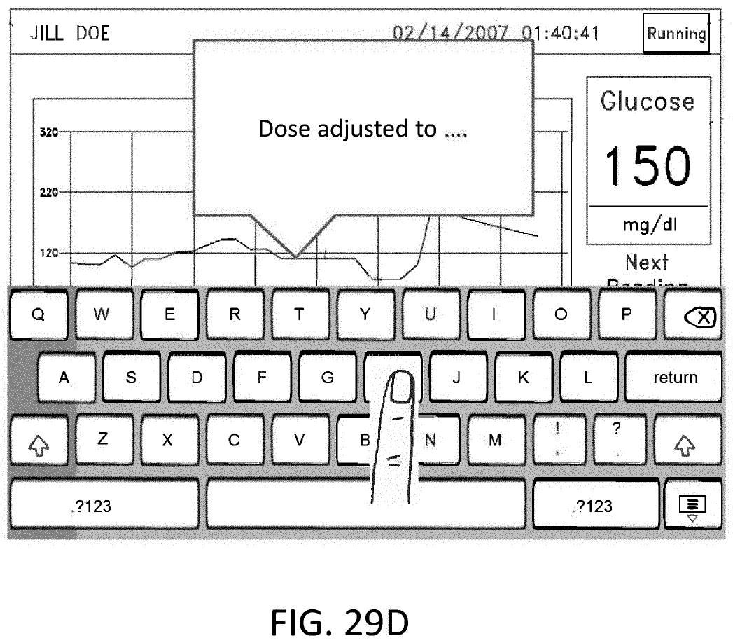

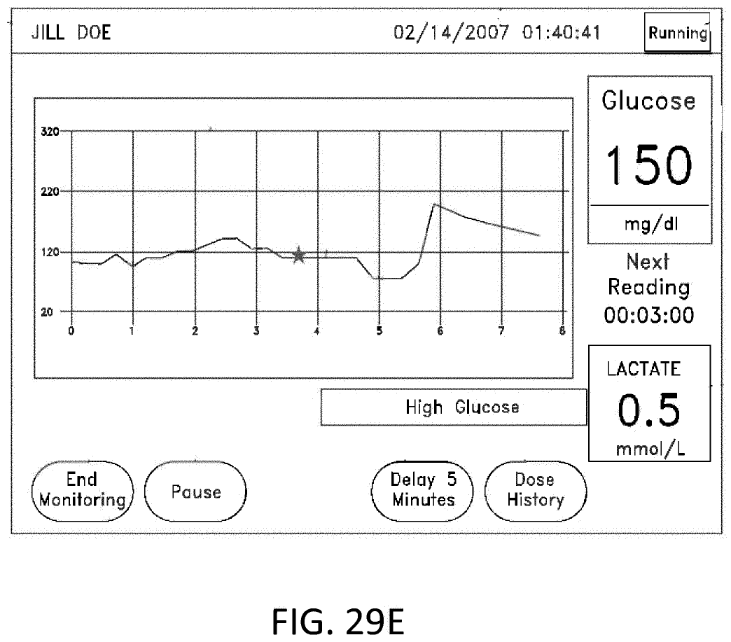

FIG. 28A shows a generalized example of a method for allowing user annotation of data. At step 2802, data can be established (e.g., collected, measured, saved, presented, recalled, displayed, confirmed, etc.). Data can be established by the systems and methods of determining physiological parameters as described herein, for example, as described in sections relating to the spectroscopic system, optical measurement, and analyte analysis of this disclosure. Data can include physiological information, system parameters, operating parameters, or other information. The data establishing step may include establishing data related to a single sample or multiple samples. At step 2804, data can be arranged. The arranged data can include data from memory and/or data related to a current or recently established parameter. Data may be arranged according to time, e.g., arranged by chronological intervals; according to type, e.g., by parameter type, measurement type, or method of analysis; or according to other variables allowing for convenient processing of data, including visual processing. At step 2806, data can be conveyed. Data may be conveyed using a user interface, for example, as described in section relating to the user interface of this disclosure, including FIGS. 24 and 25. Data may further be conveyed according to embodiments described in FIGS. 29-49 herein.

At step 2808, annotation can be allowed. Annotation can be made by selecting one or more data points. A data point may include a physiological parameter, a system parameter, or any other piece of information. Data points associated with an element of patient care may be desirable data points for selection, e.g., a data point generated at a time when a measurement was taken or a medication was administered. Data points may be selected using an input device, e.g., a keyboard, mouse, or stylus. In an embodiment, data points may be selected by on a touch screen device by touching the graphically displayed data point. An annotation or note may be created. An annotation may include information input by the user, including typed or written words, audio data, visual data including shorthand or graphics, and/or data created using tools, e.g., voice recognition software. The annotation can be displayed on a user interface while the annotation is created. In some embodiments, the annotation may be created prior to the selection of a data point and/or the data point may be selected prior to the creation of the annotation.

At step 2810, the annotation can be recorded. The annotation can be recorded in a manner that associates the annotation with the selected data point or another data point related to the selected data point. The selected data point may be a time, a physiological parameter, a patient identifier, similar piece of information, or combination or range of such information. A selected data point may be related to other data points. For example, a selected physiological parameter may be related to a data point storing the time that the physiological parameter was generated; a data point storing a different physiological parameter related to the selected physiological parameter by time, level, or other relevant link; or a data point associated with rate of change for the physiological parameter.

At step 2812, retrieval of the annotation can be facilitated. Retrieval can be facilitated by using audio and/or visual cues. Retrieval facilitated by visual cues can be performed by displaying a graphic associated with the annotation, the selected data point, or the related data point. The graphic may be an icon, a color, or other static visual indicator. The visual cue may also be intermittent, for example, a blinking display point or graphic, or dynamic, for example, an icon that appears when the user interacts with certain data or when a reminder window is temporarily displayed. Retrieval facilitated by audio cues can be performed by an audible beep, alarm, or audio recording. Audio cues may be intermittent or dynamic, for example, an audio cue may sound when the user interacts with certain data. Retrieval can be facilitated by allowing the user to interact with the provided cue, for example, by touching, clicking on, or otherwise interacting with an icon. The retrieval can be facilitated by retrieving an annotation automatically following an interaction with an audio or visual cue.

FIG. 28B shows an example of a method for allowing user annotation of data, which can be an example of the generalized method of FIG. 28A. At step 2822, analyte levels, e.g., glucose levels, can be measured at time intervals, e.g., fifteen minute intervals. Each measurement of an analyte level can establish data. The data may include the analyte level, a confidence level or error level, data associated with an incomplete measurement, or similar information. At step 2824, the analyte level or similar data may be associated with or arranged according to a time at which the measurement of the analyte level occurred. At step 2826, the analyte level and time may be displayed. The analyte level and time may be displayed on a graphical interface using a graph, for example, displaying time on the x-axis and analyte level on the y-axis. The analyte level and time can be displayed with other data, for example, by including other points on the graph representing analyte levels measured at different times.

At step 2828, annotation can be allowed by allowing a finger touch on a touch screen displaying the graphical data. Certain data points may be displayed to prompt the user to select one or more of the certain data points, for example, by highlighting the data point associated with the most recent glucose level measurement on the glucose level versus time graph. A highlight may be accomplished by displaying a color, button, or other visual cue for permitting input. When the finger touches the touch screen, one or more data points may be selected. A bubble or input screen may appear on the touch screen. The input screen can include a virtual keyboard display or an area for writing letters using a finger, a stylus, or other tool. The user may enter an annotation using the input screen. When the annotation is complete, the user may close the input screen.

At step 2830, the annotation can be recorded and a symbol can appear on the selected data. The symbol may appear as an icon next to the selected data point on the glucose level-time graph. The symbol may be a note pad icon, a check mark, a star, or other indication that a note has been made on the selected data point. At step 2812, the selected data point or the symbol next to the selected data point may be selected by a finger press on a touch screen. The note may appear in a text bubble or other enlarged format. A button or icon may appear with the enlarged text to allow the user to edit or delete the annotation.

FIGS. 29A-29E show a graphical user interface and a user selecting data for annotation, an input screen appearing on the user interface, the user entering an annotation, and a symbol appearing on the user interface for retrieval of the annotation.

FIG. 29A shows a graphical user interface according to FIGS. 24 and 25 that may be used for annotation of data. The display system may comprise an input device including a touchscreen display. The user interface 2400 may show patient identification information, including a patient name; the current date and time; an operating graphic; one or more analyte concentration graphics; a next-reading graphic; an analyte concentration status graphic; one or more trend indicators comprising a graph of an analyte concentration versus elapsed time with a trend line; and one or more buttons. The trend indicator graph shows the concentration of glucose in mg/dL units over elapsed time in hours.

FIG. 29B shows a graphical user interface and a user selecting data for annotation. The user can select data associated with the trend line representing a glucose concentration at a time, e.g., the elapsed time of 3.75 hours. The time can be measured using various units, intervals, or terms, e.g, by hours, minutes, 15 minute intervals, or irregular intervals. The time can be displayed in absolute terms, e.g., a time of day such as 12:45 p.m; in relative terms, e.g., displaying the number of time units after patient monitoring began or displaying the number of time units prior to the current time; or by associated terms, e.g., displaying the sample number in the order in which the samples were taken. The user can touch the touch screen at the trend line at the data point corresponding to the desired time, e.g., elapsed time 3.75 hours.

FIG. 29C shows a graphical user interface and an input screen appearing on the user interface. When the user's finger touches the touch screen or after a delay, an input screen may appear on the touch screen. In an embodiment, the input screen may appear as a bubble visually connected to the selected data point, e.g., the point on the trend line representing a glucose concentration at the elapsed time of 3.75 hours.

FIG. 29D shows a graphical user interface and a user entering an annotation. The input screen can include an area of the screen for inputting information, e.g., a virtual keyboard display. The virtual keyboard may be displayed with an area to view information as it is inputted, e.g., the bubble on the input screen. In some embodiments, the virtual keyboard may appear immediately with the bubble, appear after a delay, appear when the user touches the bubble, or similar. The user may enter an annotation using the virtual keyboard display, e.g., by touching the touch screen at the keyboard using a finger or a stylus. An annotation may include, for example, data about a medication dosage. When the annotation is complete, the user may close the virtual keyboard and/or the bubble to exit the input screen.

FIG. 29E shows a graphical user interface and a symbol appearing on the user interface for retrieval of the annotation. Retrieval of the note can facilitated by an icon or highlight, e.g., a star icon, appearing on the trend line at the selected data point, e.g., the point on the trend line representing a glucose concentration at the elapsed time of 3.75 hours. The user may access the note by touching the touch screen at the star on the trend line.

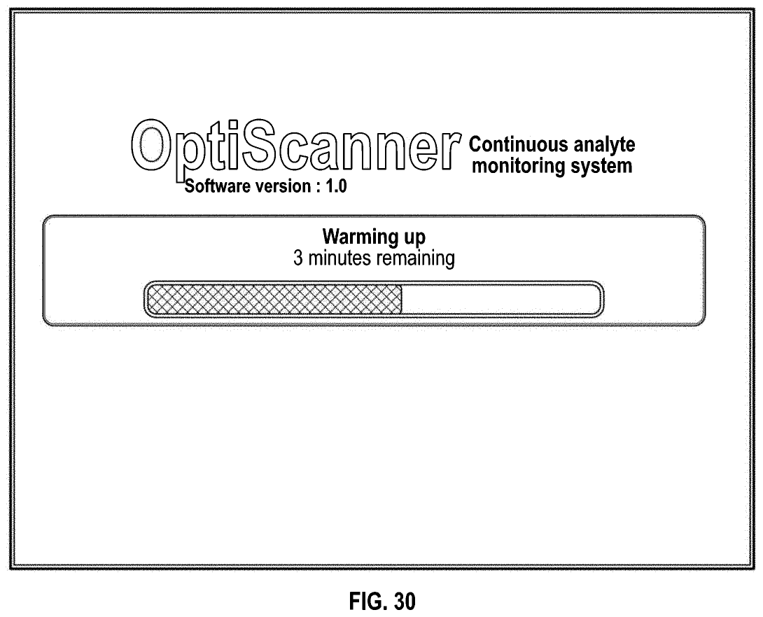

FIGS. 30-49 show screenshots of a graphical user interface of a system as described herein. A commercial embodiment according to this disclosure can be used in the OptiScanner® products, including the OptiScanner® Continuous analyte monitoring system and may be integrated into or compatible with the OptiScanner® software (e.g., version 1.0). A commercial embodiment can include some of, all of, or none of the graphical displays included in FIGS. 30-49.

FIG. 30 shows a loading screen of a graphical user interface of a system as described herein. The loading screen can include an estimate of a loading or warm-up time for the system. The loading screen can include a progress bar representing the elapsed and/or remaining loading time for the system.

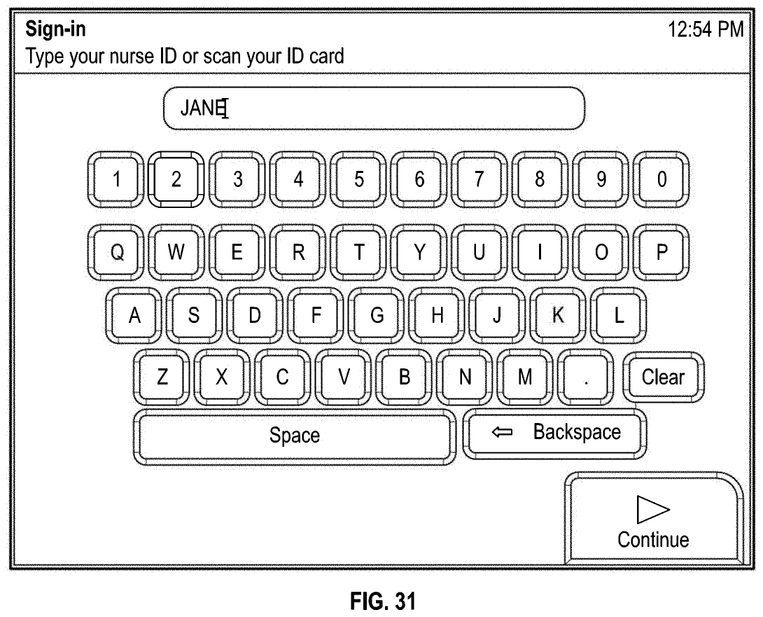

FIG. 31 shows a log in screen of a graphical user interface of a system as described herein. The log in screen may include a virtual keyboard for use with a touch screen input. The log in screen may require the user to enter a user identification code, e.g., a nurse ID, or to scan user identification material, e.g., an identification card with a magnetic strip, a bar code, or a QR code.

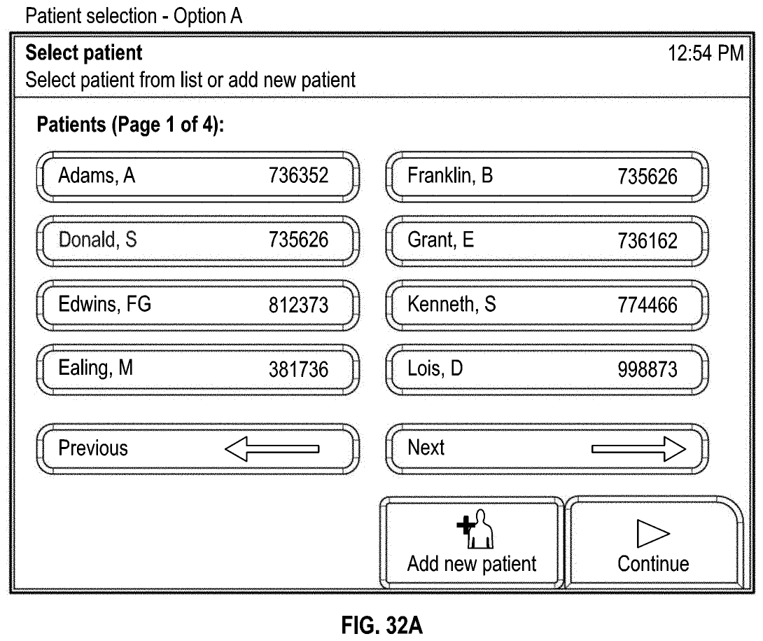

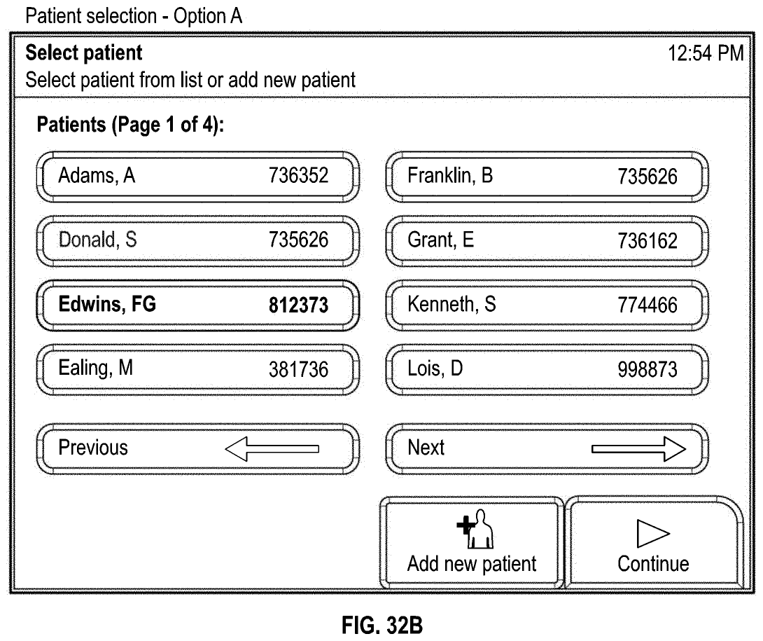

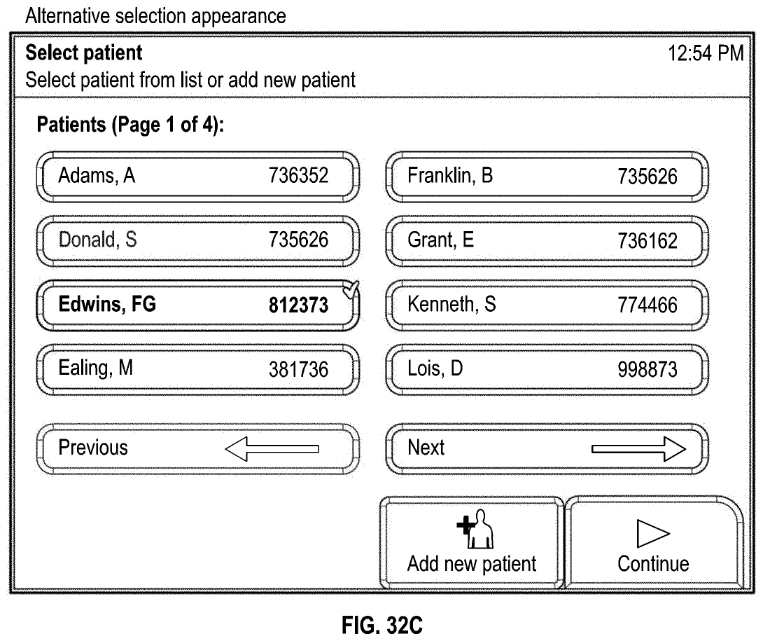

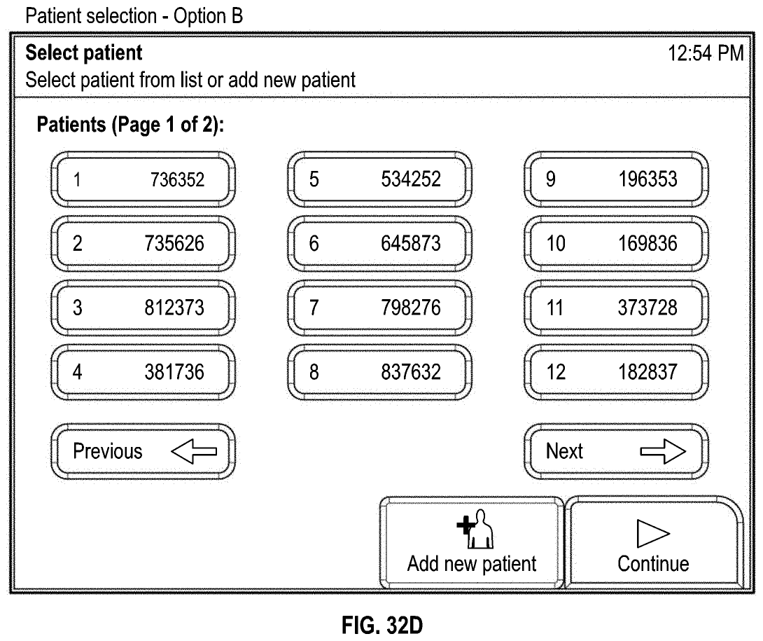

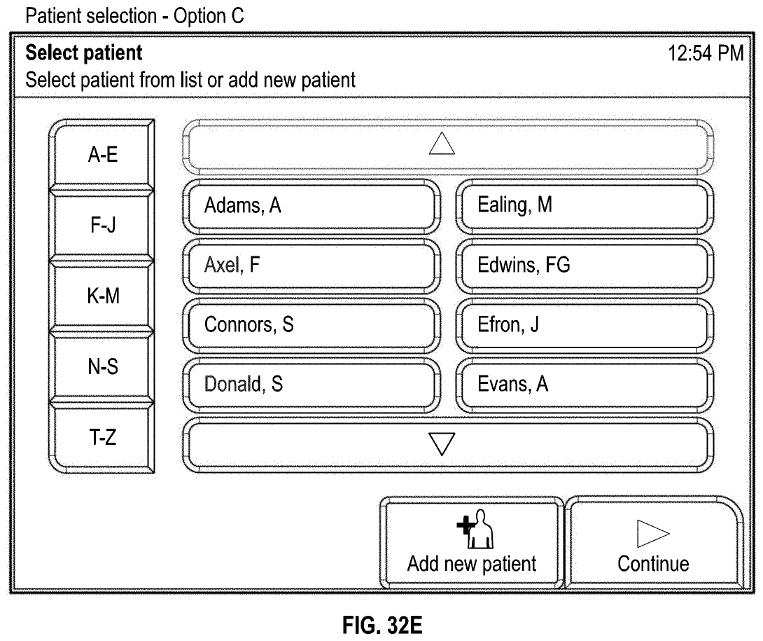

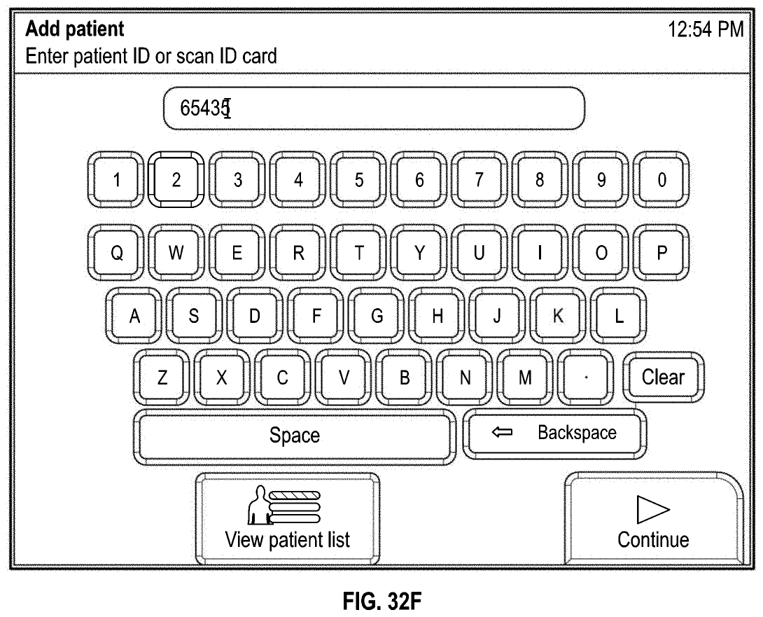

FIG. 32A shows a patient selection screen of a graphical user interface of a system as described herein. The patient selection screen can include buttons for use with a touch screen input. The buttons may include one or more buttons associated with a patient name and/or identification code. The buttons may also include arrow buttons to display additional buttons or a scroll bar. The user may navigate to additional buttons or names by swiping across or tapping areas or points on the touch screen. The buttons may also include buttons to navigate to screens allowing the user to add patient information or monitor patient data. A button to continue to the patient monitoring screen may be disabled or inactive and may become available when a button associated with a patient is selected. The screen may also include a header with information, e.g., instructions, identification of the system or screen, and/or a date or time.

FIG. 32B shows a patient selection screen of a graphical user interface of a system as described herein. A button associated with a patient name and/or identification code has been selected, as shown by the graphical indentation of the button associated with a patient name “Edwins, FG” and the different color of the button. Various visual cues may be used to show selection of a button associated with a patient name, e.g., highlighting, color change, a display icon, and similar cues. The continue button in the bottom right-hand corner of the display may be enabled as shown when a patient is selected.

FIG. 32C shows a patient selection screen of a graphical user interface of a system as described herein. A button associated with a patient name has been selected. An icon, e.g., a check mark, may be displayed on or next to the button. The icon may indicate that an annotation has been made in the data associated with the patient.

FIG. 32D shows a patient selection screen of a graphical user interface of a system as described herein. The patient selection screen can include buttons associated with a patient identification code. As described in FIGS. 32A-32C, the buttons may also include navigation buttons, scroll bars, or other information displayed on the screen.

FIG. 32E shows a patient selection screen of a graphical user interface of a system as described herein. The patient selection screen can include buttons associated with a patient name. As described in FIGS. 32A-32C, the buttons may also include navigation buttons, scroll bars, or other information displayed on the screen. The navigation buttons may also include buttons with classification data, e.g., an alphabetically letter or range of letters, to assist with navigation through the list of patient names and/or identification codes.

FIG. 32F shows a patient addition screen of a graphical user interface of a system as described herein. The patient selection screen can include a button associated with adding a new patient to the system. The patient addition button may navigate to a patient addition screen. The patient addition screen may include a virtual keyboard for use with a touch screen input. The log in screen may require the user to enter a user identification code, e.g., a patient ID, or to scan user identification material, e.g., an identification card with a magnetic strip, a bar code, or a QR code. The patient addition screen may include a continue button to complete the process to adding a patient to the system or to continue to additional steps in the process of adding a patient to the system.

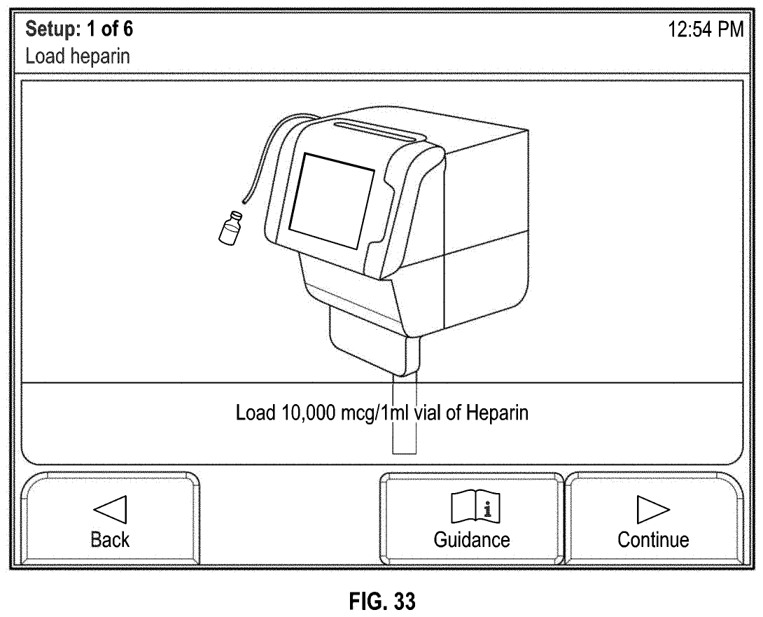

FIG. 33 shows an instruction screen of a graphical user interface of a system as described herein. The instruction screen may be displayed after a patient is selected. The instruction screen may include other information, e.g., reference or navigation information. Some examples of reference or navigation information can include a header, graphics, and text with patient care instructions, for example, instructions to “Load 10,000 mcg/1 ml vial of heparin” into the fluid handling system. The instruction screen may include additional buttons or information, e.g., a button for additional instructions or information.

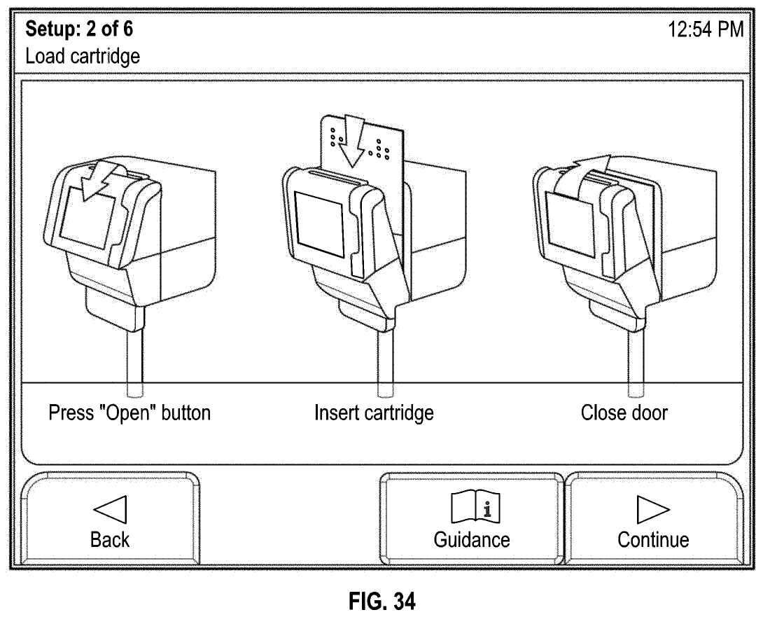

FIG. 34 shows an instruction screen of a graphical user interface of a system as described herein. Additional instruction screens may be displayed after a patient is selected. The additional instruction screens may include additional steps related to one or more patient care steps to be executed. The instruction screen may include a header, graphics, and text with patient care instructions, e.g., instructions comprising steps following a previously taken patient care step.

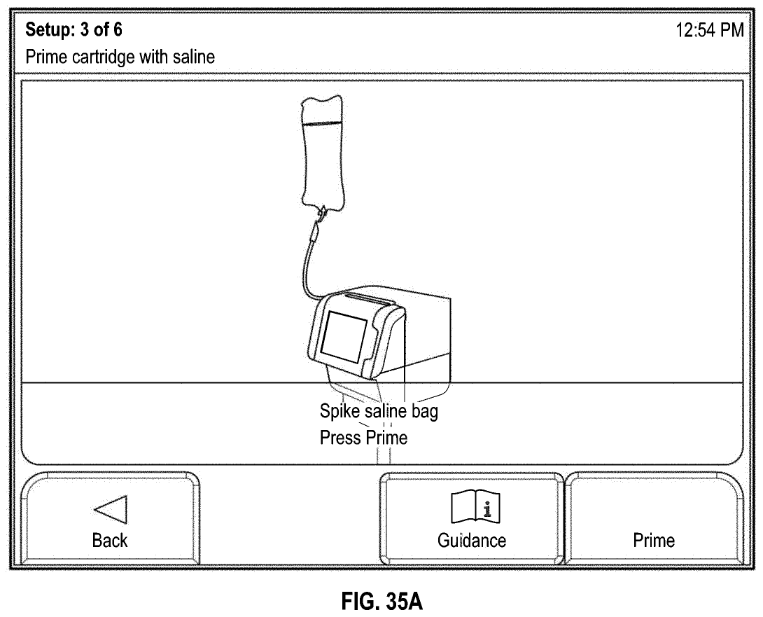

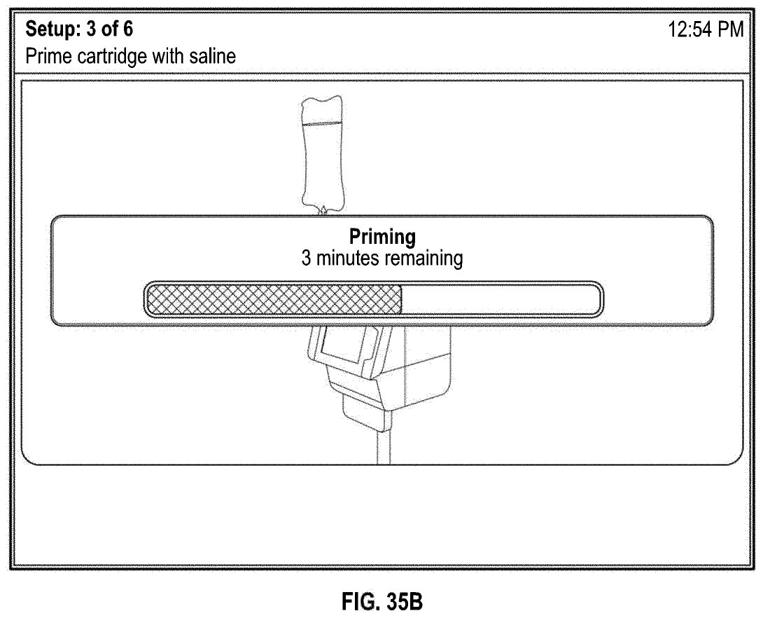

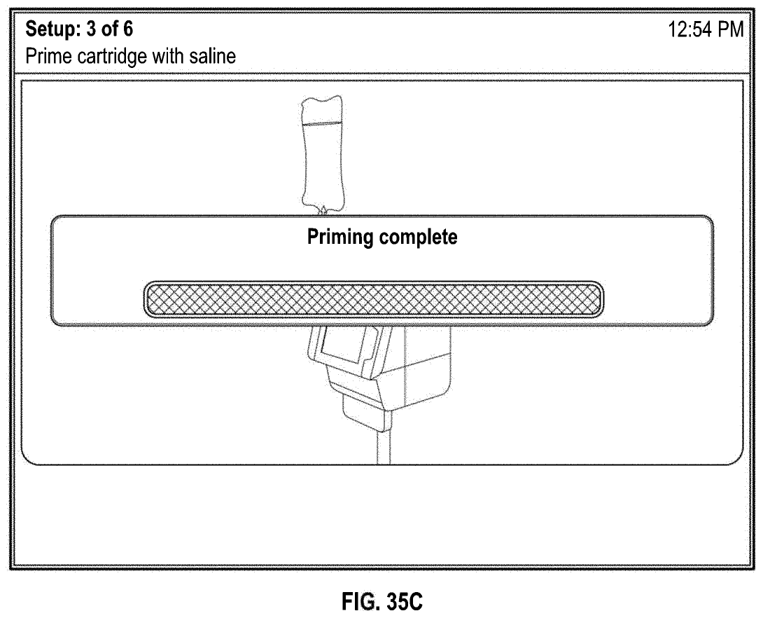

FIG. 35A shows an instruction screen of a graphical user interface of a system as described herein. Additional instruction screens may be displayed after a patient is selected. The additional instruction screens may include additional steps related to one or more patient care steps to be executed. The instruction screen may include a header, graphics, and text with patient care instructions, e.g., instructions comprising steps to operate the patient monitoring system. The buttons displayed on the screen may include text or graphics associated with the specific patient care or system operation step to be executed.

FIG. 35B shows an instruction loading screen of a graphical user interface of a system as described herein. Additional instruction loading screens may be displayed after specific patient care or system operation steps are performed. The instruction loading screen can include an estimate of a loading time for the system. The instruction loading screen can include a progress bar representing the elapsed and remaining loading time for the system.

FIG. 35C shows an instruction completion screen of a graphical user interface of a system as described herein. An instruction completion screen may be displayed after a specific patient care or system operation step is performed and completed. The instruction completion screen can include a progress bar representing the elapsed, completed loading time for the system.

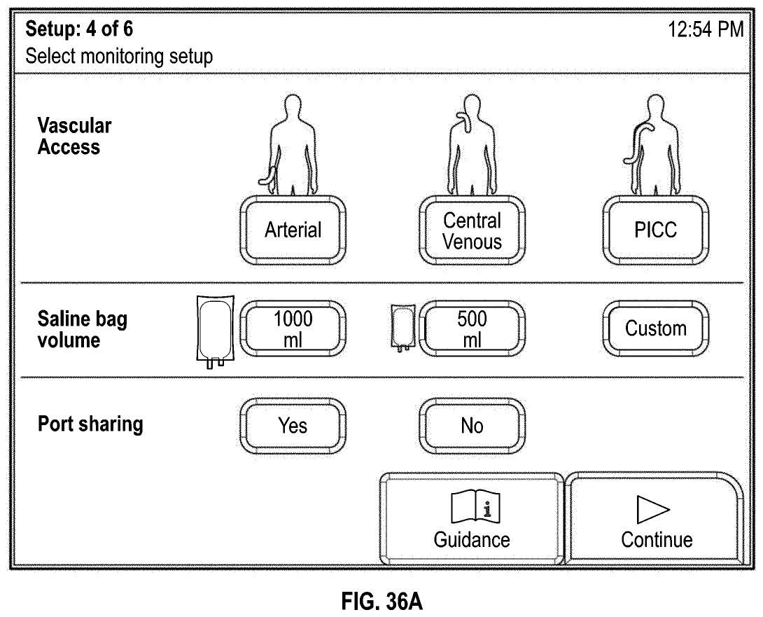

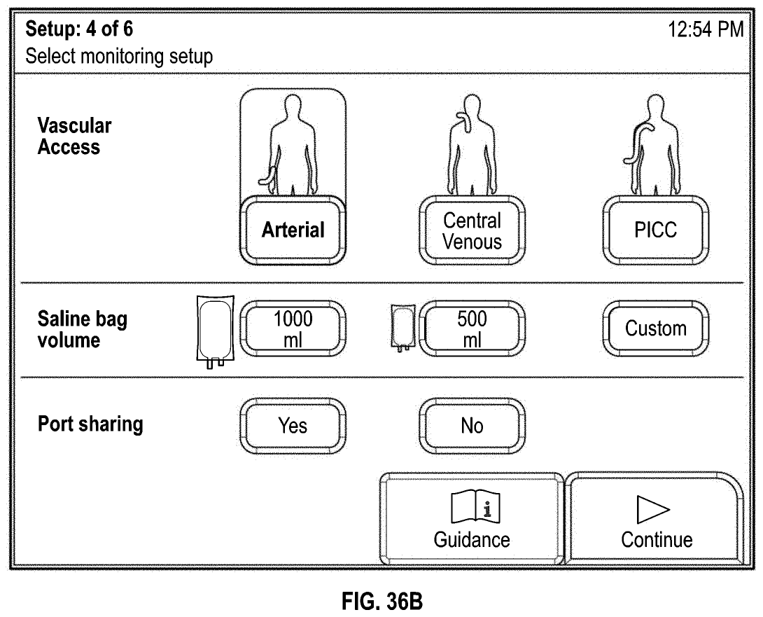

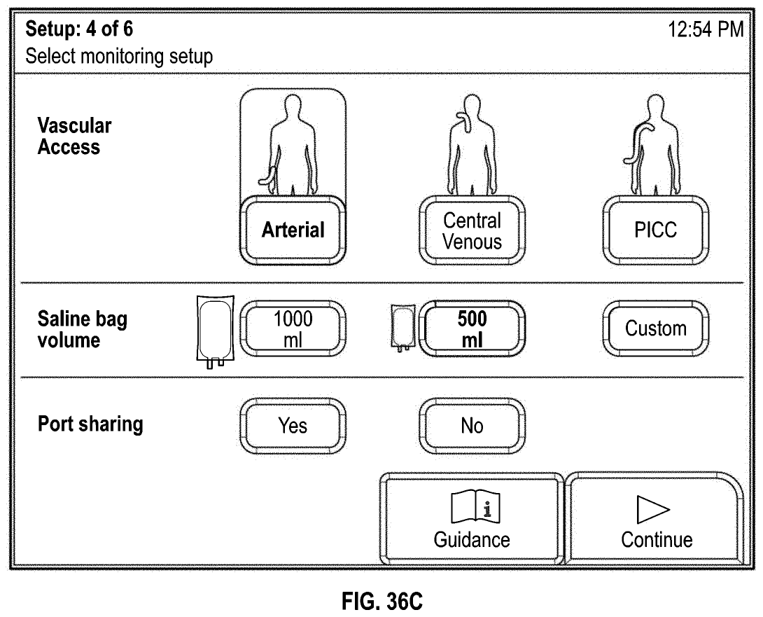

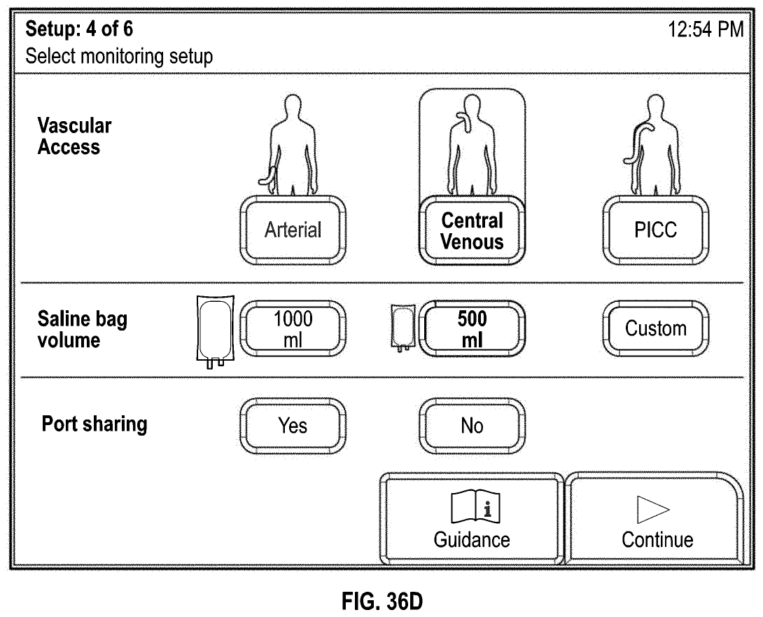

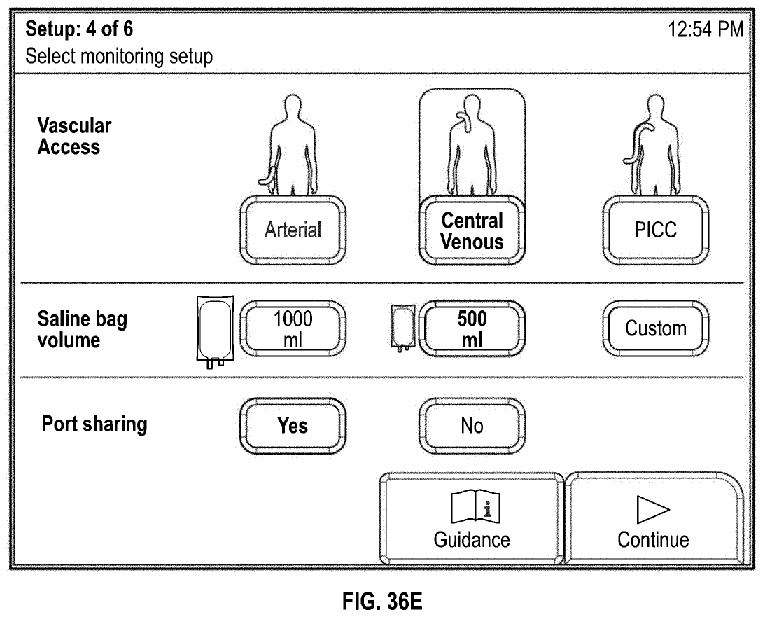

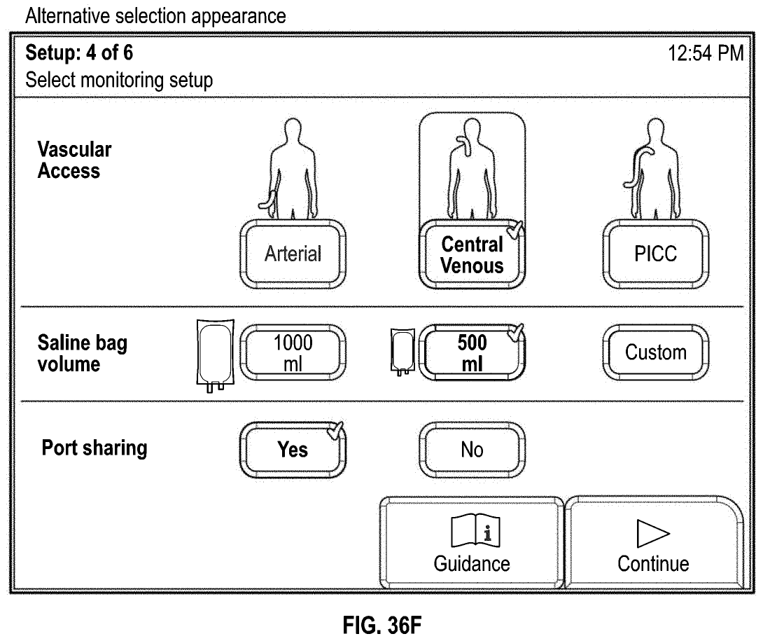

FIG. 36A shows an instruction screen of a graphical user interface of a system as described herein. Additional instruction screens may be displayed after a patient is selected. The additional instruction screens may include additional steps related to one or more patient care steps to be executed. The instruction screen may include a header, graphics, and text with patient care instructions, e.g., instructions to select a method of vascular access used to provide heparin to a patient. The buttons displayed on the screen may include text or graphics associated with the specific patient care or system operation step to be executed, e.g., dosage amounts, access types, or port sharing information.

FIGS. 36B-36E show screenshots of an instruction screen of a graphical user interface of a system as described herein. Additional instruction screens may be displayed after a patient is selected. Buttons associated with information or selections displayed on the instruction screen may be selected or highlighted.

FIG. 36F shows an instruction screen of a graphical user interface of a system as described herein. Additional instruction screens may be displayed after a patient is selected. Icons may appear on the screen on or next to buttons associated with information or selections displayed on the instruction screen. Icons, e.g., check marks, may indicate that an annotation has been made or stored and may be retrieved. The annotation may be related to the patient care, system operation step, or other information that may be useful for annotation indicated by the instruction screen. The annotation may be accessed by touching or selecting the icon.

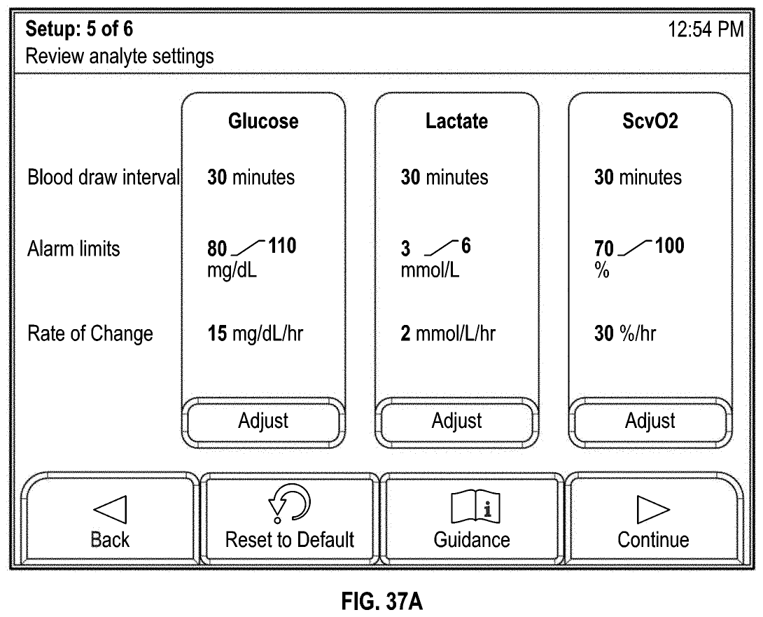

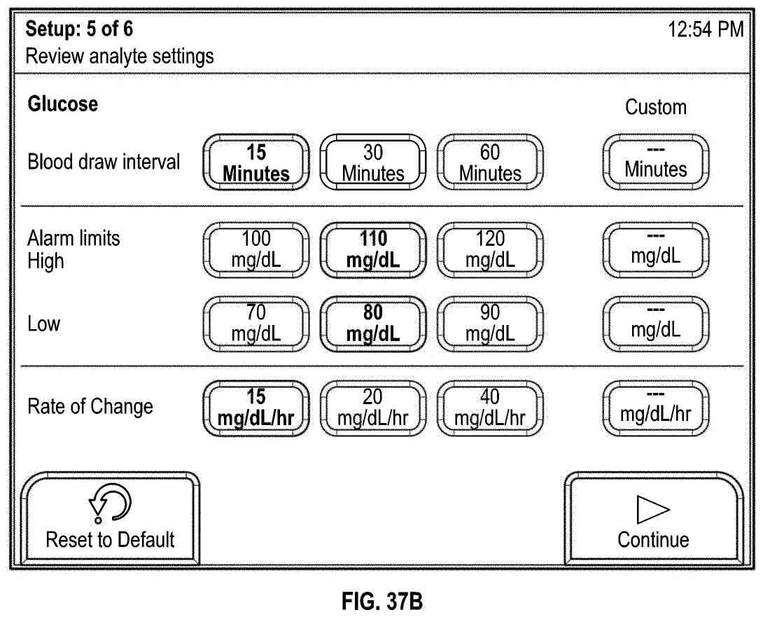

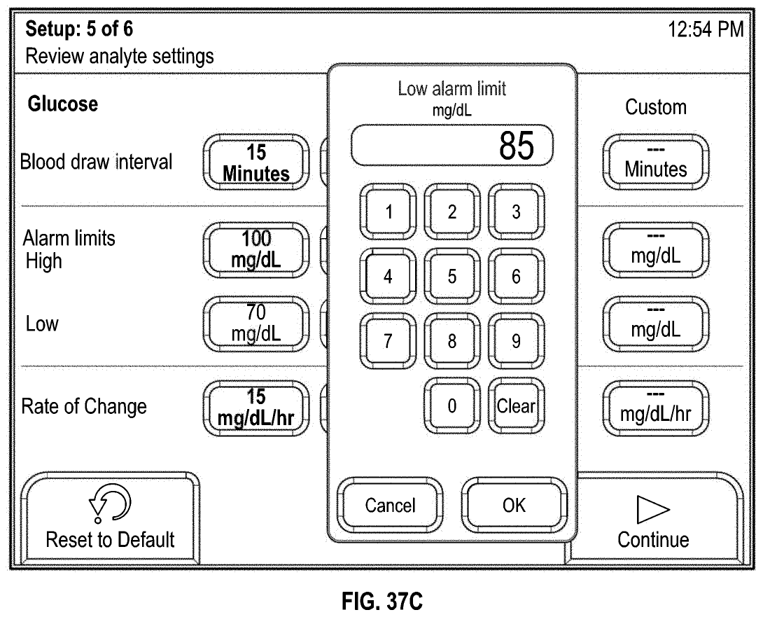

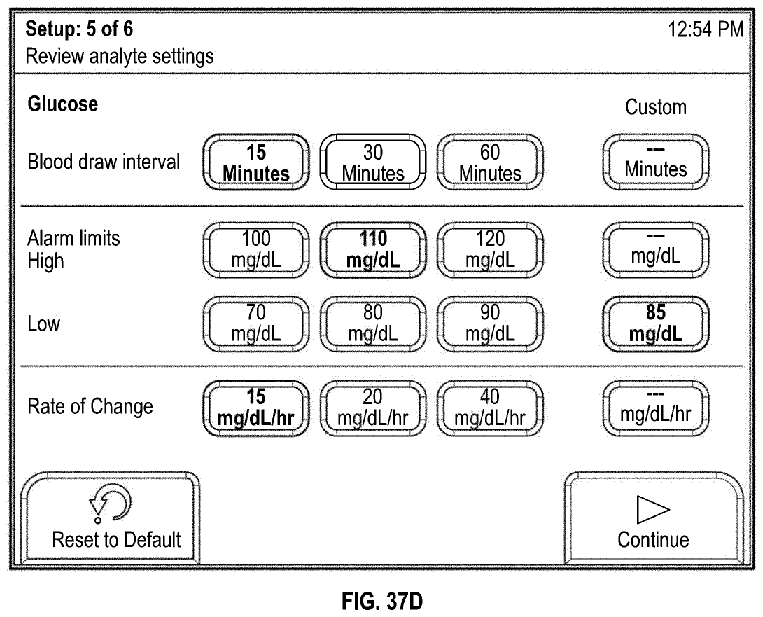

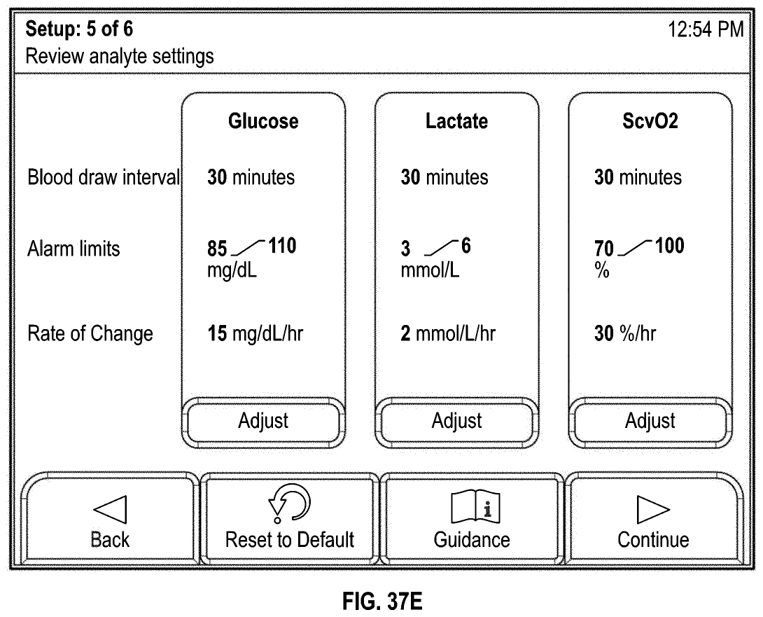

FIGS. 37A-37E show screenshots of an instruction screen of a graphical user interface (e.g., user interface 2400) of a system (e.g., patient monitoring system 2630, system 400) as described herein. Additional instruction screens may be displayed after a patient is selected. The additional instruction screens may include additional steps related to one or more patient care steps to be executed. The instruction screen may include a header including graphics, and text with patient care instructions, e.g., instructions to review analyte settings for a patient including glucose, lactate, and/or ScvO2. Additional information relating to the instruction may appear on the screen. The buttons displayed on the screen may include text or graphics associated with the specific patient care or system operation step to be executed, e.g., blood draw intervals, alarm limits, or rate of change information. An adjust button or touching the text or graphics may load an input screen or bubble, allowing the user to change patient care information. Custom levels may be entered using an input screen and virtual keyboard, or by using arrows or scrolls to change numerical levels. Predetermined levels may be selected by touching displayed buttons. After selected patient care requirements, the user may return to the analyte settings screen.

The screens shown in FIGS. 37A-37E are examples of how a user can interact with the display and adjust parameters, change alarm settings, indicate desired time windows for data display, etc. Such an interactive approach can also be applied when inserting annotations into a data record by touching or otherwise selecting a portion of a data display. In particular, the parameters of a time window for future value predictions can be adjusted and the desired rate of change for increasingly or decreasingly intense alarms or colors can be adjusted by a user (see, e.g., general discussion herein with respect to FIGS. 24-25 and 40A-49). Moreover, a warning can be annotated to indicate a health care provider's reason for minimizing, ignoring, or deferring action that might otherwise be warranted in response to an alarm or other predicted trend.

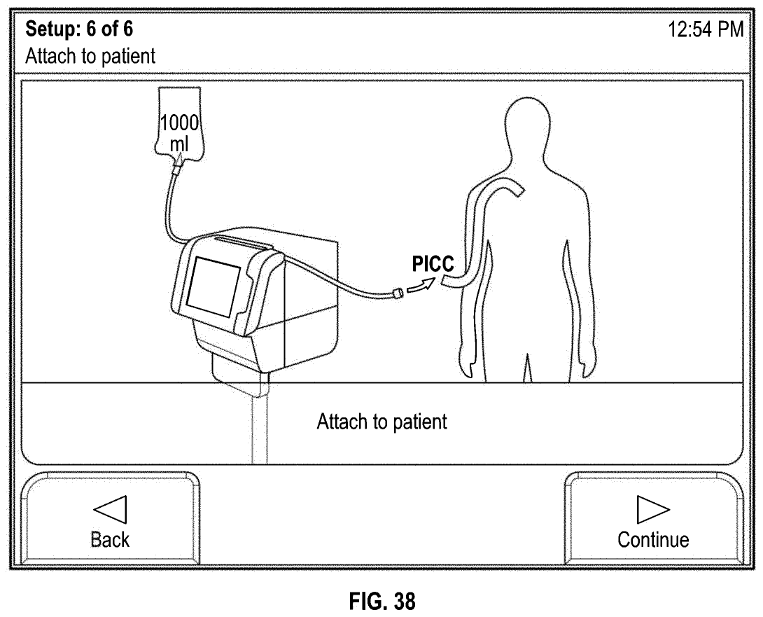

FIG. 38 shows an instruction screen of a graphical user interface of a system as described herein. Additional instruction screens may be displayed after a patient is selected. The additional instruction screens may include additional steps related to one or more patient care steps to be executed. The instruction screen may include a header, graphics, and text with patient care instructions, e.g., instructions comprising steps following a previously taken patient care step.

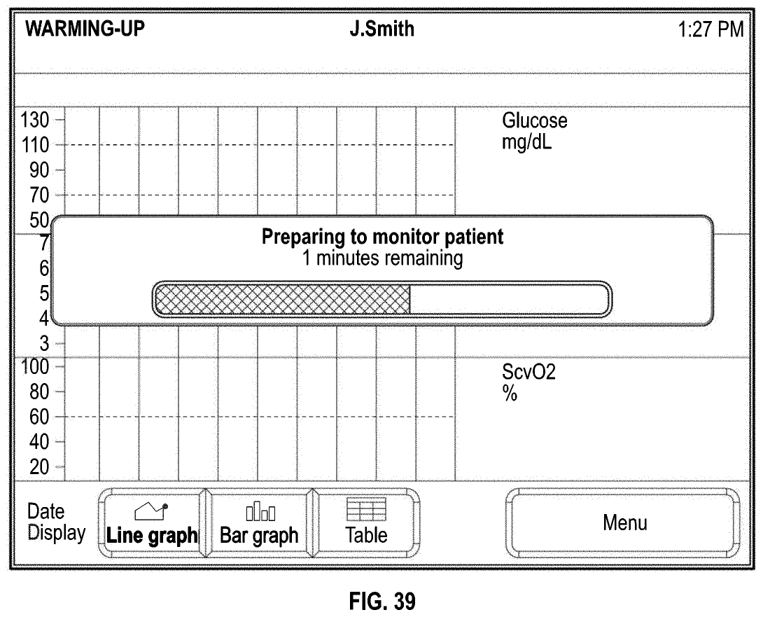

FIG. 39 shows a monitoring loading screen of a graphical user interface of a system as described herein. A monitoring loading screen may be displayed after patient care or system operation steps are completed and patient monitoring begins. The monitoring loading screen can include an estimate of a loading time for the system. The monitoring loading screen can include a progress bar representing the elapsed and remaining loading time for the system.

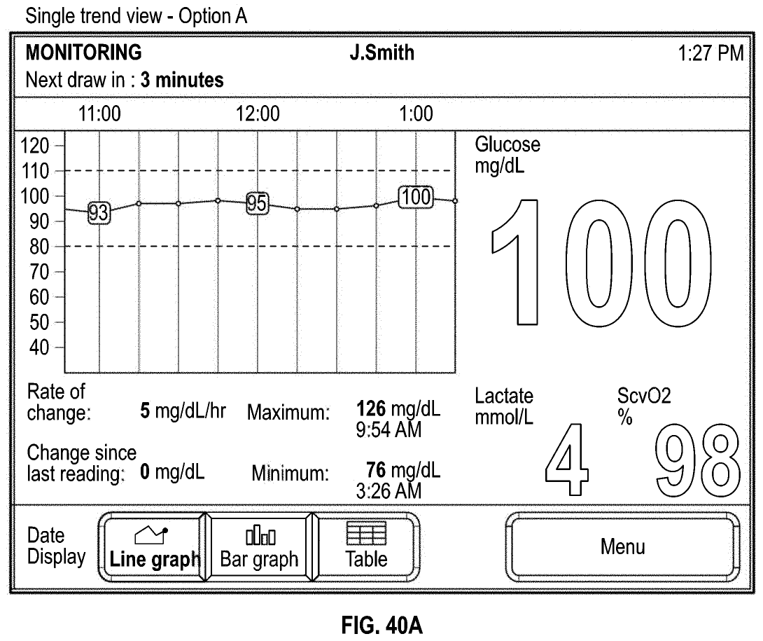

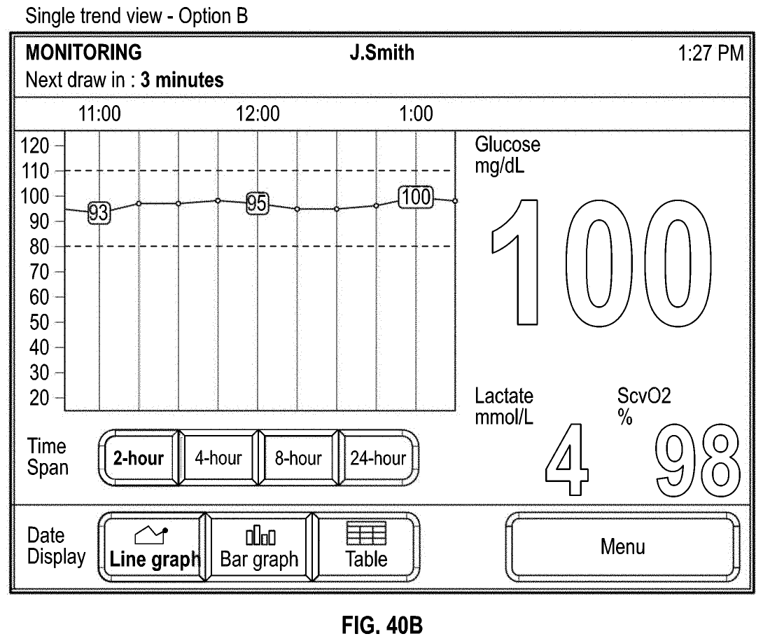

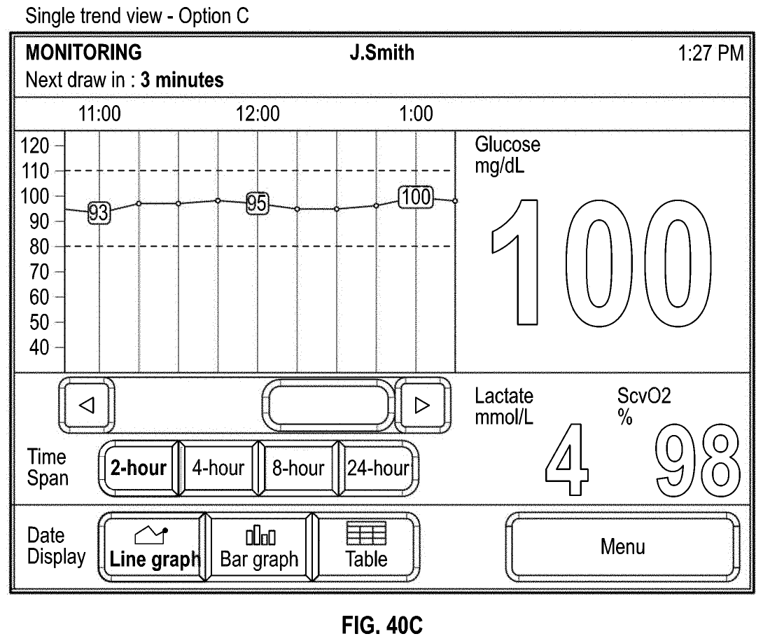

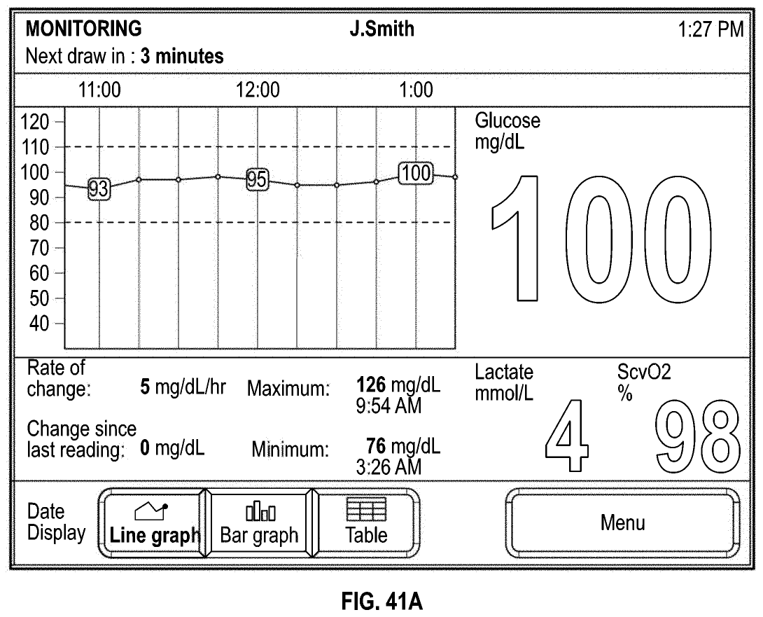

FIG. 40A shows a monitoring screen of a graphical user interface (e.g., user interface 2400) of a system (e.g., patient monitoring system 2630, system 400) as described herein. The monitoring screen may include user interface elements describe in the user interface and annotation of data sections of this disclosure, including descriptions of FIGS. 24-25 and FIGS. 29A-29E. The user interface elements may include a header with patient identification information, including a patient name; the current date and time; an operating graphic; and a next-reading graphic. The user interface elements may also include one or more analyte concentration graphics, e.g., glucose, lactate, and/or ScvO2; an analyte concentration status graphic; one or more trend indicators comprising a graph of an analyte concentration versus elapsed time with a trend line; information associated with the analyte concentration, including rate of change and minimum and maximum monitoring levels; and one or more buttons. The trend indicator graph may show the concentration of glucose in mg/dL units over elapsed time in hours. The trend indicator graph may include lines representing a minimum and maximum range for the displayed physiological parameter. The minimum and maximum range may be associated with a reference standard. One or more buttons can allow the user to navigate between types of trend indicator display types, e.g., line graph, bar graphs, tables, and/or other information included on the trend indicator display. One or more buttons may allow the user to navigate between the type of analyte concentration displayed on the trend indicator graph. The type of analyte concentration displayed on the trend indicator graph may also be changed by touched areas of the screen, e.g., by touching the analyte concentration graphic for lactate to view a trend indicator graph for lactate.

FIG. 40B shows a monitoring screen of a graphical user interface of a system as described herein. The trend indicator graph may be adjusted along the x-axis or the y-axis by unit range, e.g., by changing the time range of the x-axis between a 2 hour, a 4 hour, an 8 hour, and a 24 hour range.

FIG. 40C shows a monitoring screen of a graphical user interface of a system as described herein. The trend indicator graph may be adjusted along the x-axis or the y-axis by unit range, e.g., by changing the time range of the x-axis using a scroll bar, arrow buttons, or by touching or swiping an area of the screen.

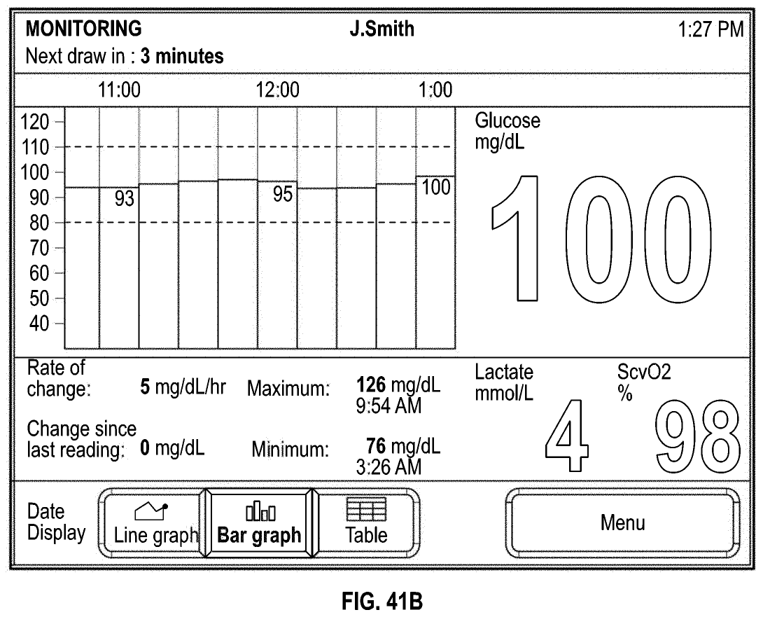

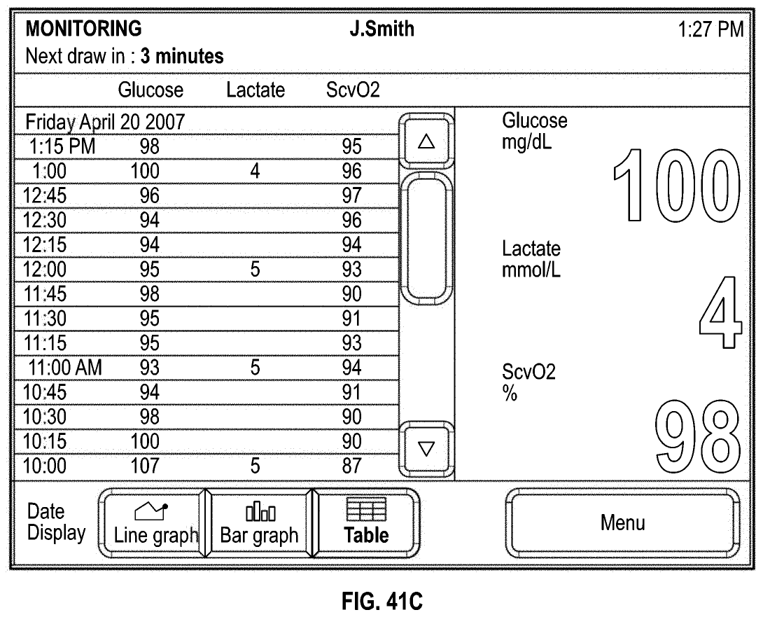

FIG. 41A shows a monitoring screen of a graphical user interface of a system as described herein. The trend indicator graph may be displayed as a line graph, with data points displayed and a trend line interpolated between the data points.

FIG. 41B shows a monitoring screen of a graphical user interface of a system as described herein. The trend indicator graph may be displayed as a bar graph, with data points displayed as filled bars up to the number corresponding to the measurement. The bar graph may also include lines representing a minimum and maximum range, e.g., a reference standard.

FIG. 41C shows a monitoring screen of a graphical user interface of a system as described herein. The trend indicator graph may be displayed as a table. The table may include data associated with each data collection, including time and each physiological parameter measured.

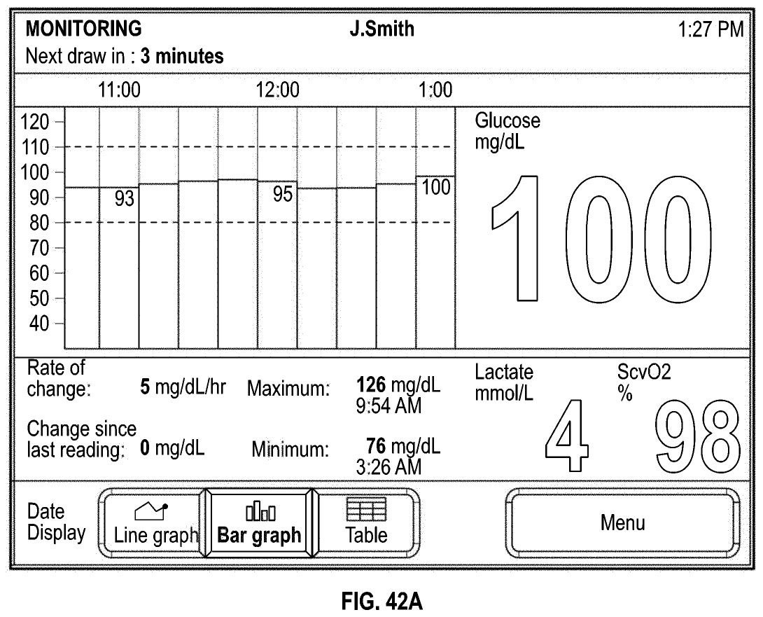

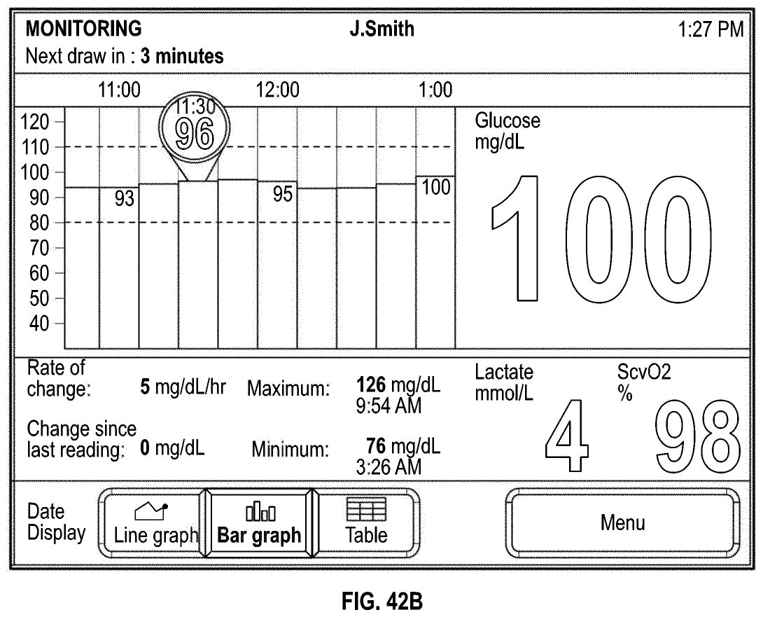

FIG. 42A shows a monitoring screen of a graphical user interface of a system as described herein. The trend indicator graph may be displayed as a bar graph, with data points displayed as filled bars up to the number corresponding to the measurement. The bar graph may also include lines representing a minimum and maximum range, e.g., a reference standard.

FIG. 42B shows a monitoring screen including data selected for annotation of a graphical user interface of a system as described herein. The trend indicator graph may be displayed as a bar graph. One or more data points may be selected for annotation by touched a bar associated with a data point. The selection of a data point may display additional numerical information corresponding to the data point. The selection of a data point may highlight the data point. In addition, after an annotation is added, a data point associated with an annotation may remain highlighted, display additional numerical information, or include an icon on or near the data point.

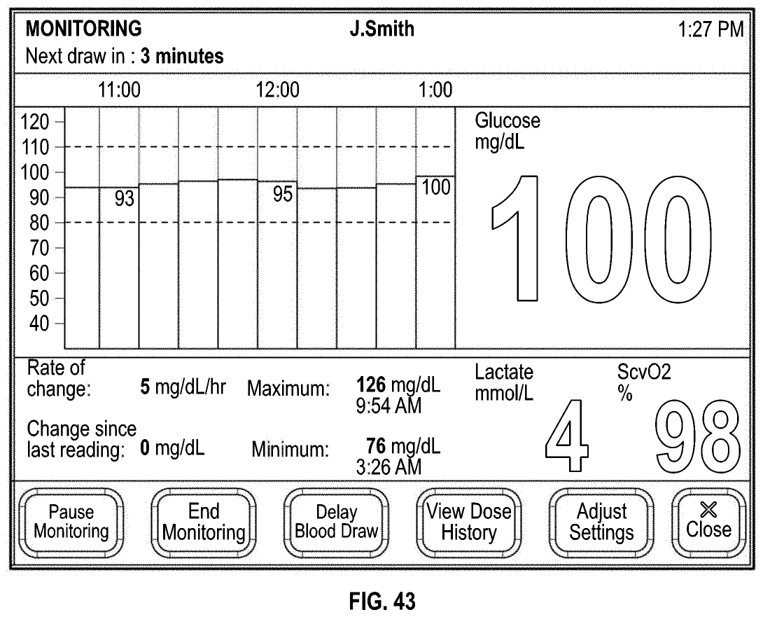

FIG. 43 shows a monitoring screen including a menu display of a graphical user interface of a system as described herein. A menu button may be selected during patient monitoring. The menu button may display additional monitoring or system options, including buttons described in FIGS. 24-25.

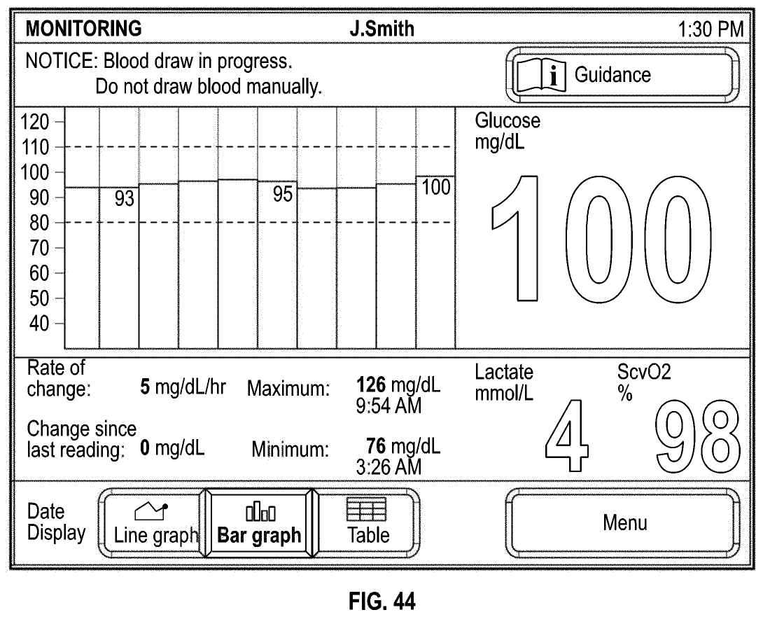

FIG. 44 shows a monitoring screen including a notice bar of a graphical user interface of a system as described herein. A notice bar or other window may be displayed during patient care or system processes. The notice bar may include warnings or instructions. The notice bar may appear in a bright or noticeable color, e.g., light blue.

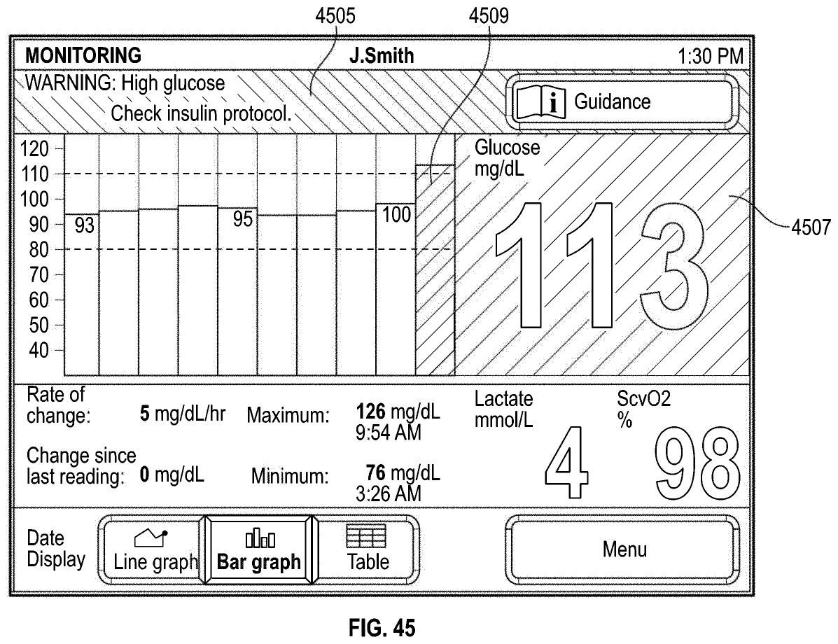

FIG. 45 shows a monitoring screen including a warning bar of a graphical user interface of a system as described herein. A warning bar 4505 or other window may be displayed when a physiological parameter exceeds or falls below a reference measure or minimum or maximum range. The warning bar 4505 may include warnings or instructions. The warning bar may appear in a high-contract color, e.g., bright red, to ensure visibility. Other sections of the display screen may be displayed in contrasting colors, e.g., the numerical indicator 4507 or the data point displayed as a filled bar 4509 up to the value of the measurement for the physiological parameter exceeding or falling below the reference standard. Other contrasting colors, e.g., a dark red, may be used on other sections of the screen. In various implementations, the monitoring screen shown in FIG. 45 can be configured to display the warning bar 4505 or a warning window if the value of the physiological parameter at a future time is predicted to exceed or fall below a reference measure or minimum or maximum range. In such instances, the warning bar 4505 or window can appear in a color (e.g., yellow or orange) different from the color (e.g., bright red or dark red) used to indicate that the physiological parameter exceeds or falls below a reference measure or minimum or maximum range.

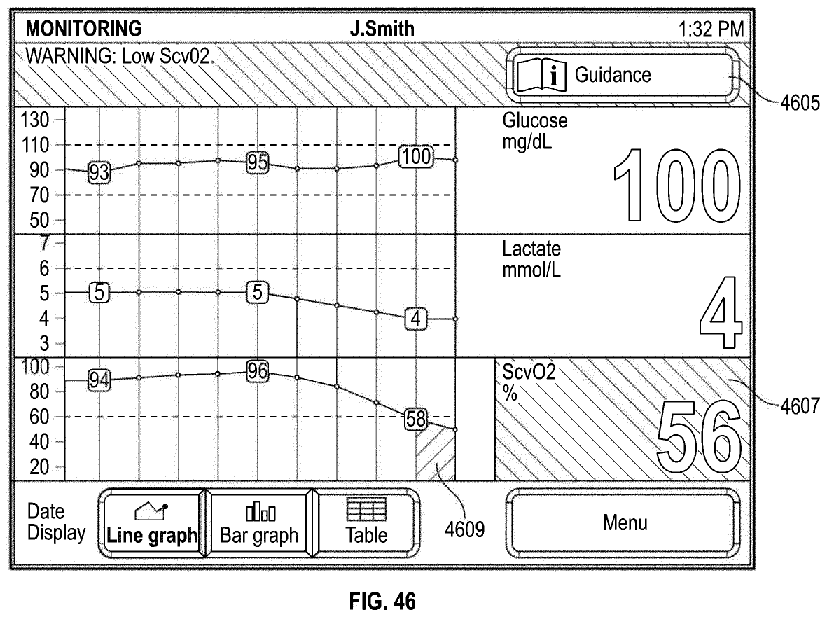

FIG. 46 shows a monitoring screen including a warning bar 4605 of a graphical user interface of a system as described herein. A warning bar 4605 or other window may be displayed when a physiological parameter exceeds or falls below a reference measure or minimum or maximum range. The user interface may change to prominently display the physiological parameter exceeding or falling below the reference standard, e.g., by displaying the numerical indicator 4607 and/or the trend indicator 4609 for the physiological parameter. The numerical indicator 4607 and/or the trend indicator 4609 may display the measurement producing the warning in a high-contrast color. The warning bar 4605 may also be displayed with an audible noise or alarm.

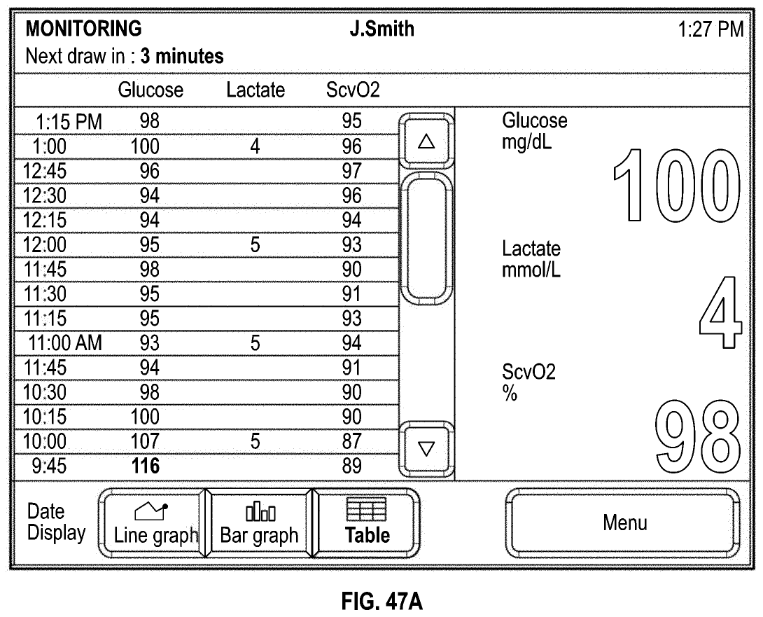

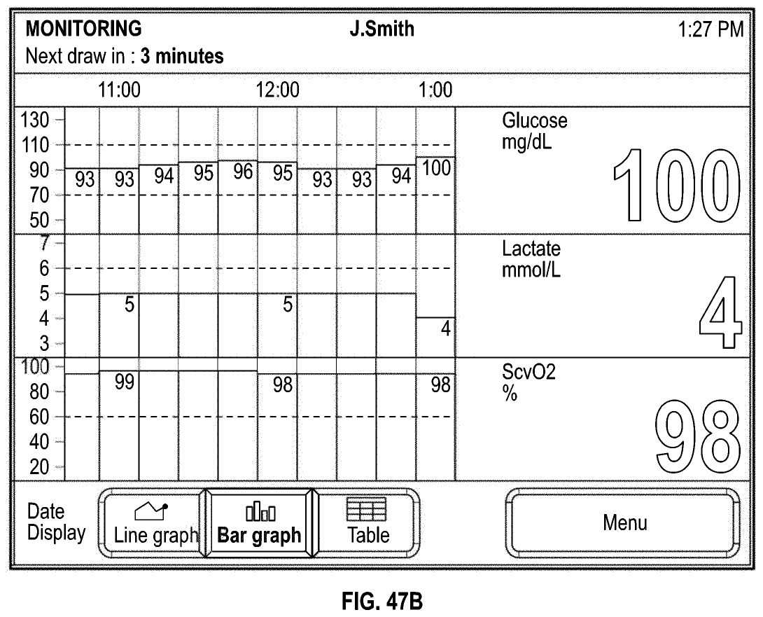

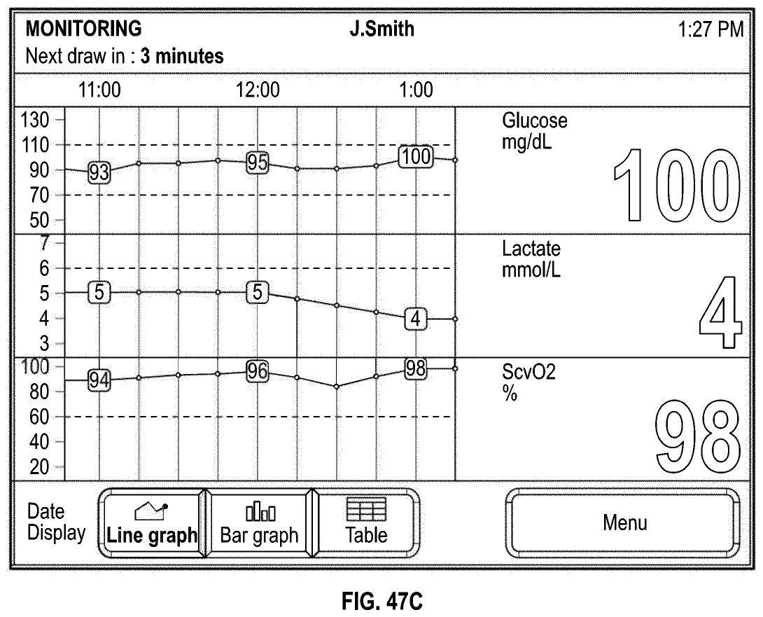

FIG. 47A shows a monitoring screen displaying a table format of a graphical user interface of a system as described herein. A table display or a trend indicator may display a past measurement producing a warning in a high-contrast color.

FIGS. 47B-47C show screenshots of a monitoring screen displaying multiple trend indicators of a graphical user interface of a system as described herein. Multiple trend indicators may be displayed on a single screen.

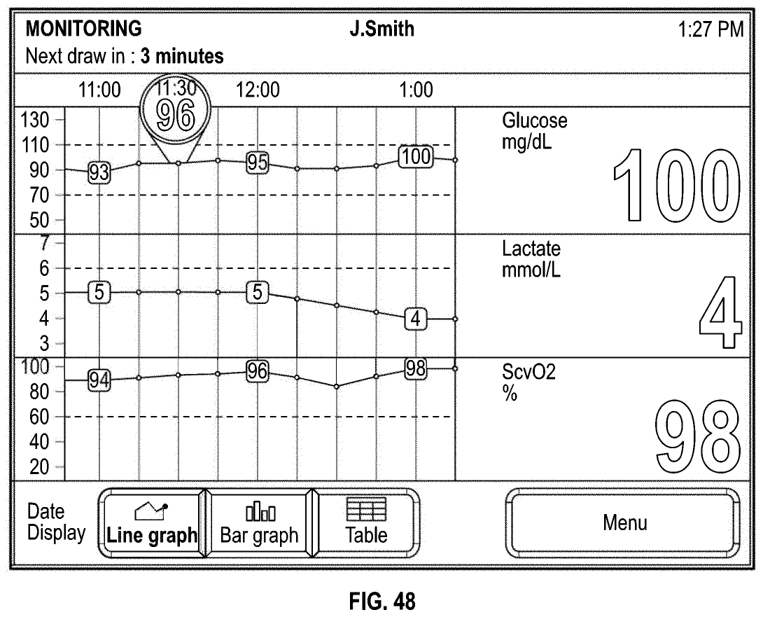

FIG. 48 shows a monitoring screen displaying multiple trend indicators of a graphical user interface of a system as described herein. Multiple trend indicators may be displayed on a single screen. A user interface displaying multiple trend indicators may be advantageous for selecting data for annotation and displaying indicators to facilitation annotation retrieval. For example, a user may select a data point associated with a specific blood analyte at a specific time for annotation. In addition, the multiple trend indicators may display annotations associated with different specific physiological parameters at different times without switching between display screens.

FIG. 49 shows a monitoring screen displaying multiple trend indicators of a graphical user interface of a system as described herein. Multiple trend indicators may be displayed on a single screen. Each trend indicator may display areas of the graph associated with a reference standard in a selected high or low contrast color. For example, areas 4905 in a small range below a minimum and/or above maximum level or reference standard may be displayed in yellow or dark yellow, and areas 4907 outside the acceptable levels of the reference standard and the small range above or below the reference standard may be displayed in red or dark red. In various implementations, the monitoring screen shown in FIG. 49 can be configured such that the trend indicator for each analyte is displayed in yellow, dark yellow or orange color if the trend predicts that the concentration of the analyte at a future time may be outside the acceptable levels of the reference standard. Color families, intensities, etc. can be used together to indicate related concepts. For example, a light yellow number value, background color, or trend line can indicate a mild warning or a predicted value that will stray from an accepted range at a far future time. A more intense yellow (higher color saturation) or a different but related hue or chroma can be used to indicate increasing or decreasing need for attention from a healthcare provider. For example, yellow can turn to orange and then red as a predicted danger becomes more extreme or more imminent. Additional levels of alarm can also be included, such as flashing, beeping, haptic alarms, etc. These principles apply not only for trend lines but for other styles of data display as well (including tabular and bar graph displays, status graphics 2408 and 2412, etc.

In various implementations, the monitoring screens shown in FIGS. 40A-49 can display a predicted value of the concentration at a future time. This can be accomplished by displaying a separate number (e.g., in a smaller font, different color, separate location, etc.). FIGS. 40A-49 do not show this separate value, but such a separate display can be included. In addition to displaying a predicted future value separately from the presently measured value, predictions and projections can be indicated using a trend line that is extrapolated forward but displayed in a dimmer color to indicate some statistical uncertainty. Some approaches can use dotted lines that continue along a previously-established trajectory, and the dotted line can indicate or be associated with a predicted value. Although in FIGS. 40A-49, the present time is at the far right or lower end of the graphical or tabular data, if predicted values are displayed, the present time can be shifted to the left (or shifted upward in the table) to allow room at the right for future predictions. These principles can also apply to the other graphical presentations of data—the bar and table approaches, for example, and not just the line graph approach.

Many other options exist for annotating data (e.g., through interaction with a display or otherwise). For example, data can be automatically annotated with data from other sources such as a camera recording activities relating to the vicinity of a patient, automatic recordation of dosage amounts, read-outs from other monitors such as heart-rate, other oxygen monitors, etc. Data can be conveyed aurally. Whether data is conveyed visually, aurally, or otherwise, notes can be recorded using sound. For example, a microphone and voice recognition technology can be used to record a physician's voice saying: “note at the last data point: dose was increased by 3 milligrams here”; or “target level changed in view of test data from Dr. Jones' lab”; etc.

Data can be annotated using a variety of technological systems, including recordings, e.g., still photographs or scans, of patient data generated outside the patient monitoring system. Various technologies may be used to assist visual recording, including character recognition technology or digital writing instrument transcription. For example, data may be annotated by selecting a data point, using a camera connected to a network interface to take a photograph of a patient chart, transferring the photograph to the patient monitoring system or other patient data network, and associating the photograph with the data point. Character recognition may be performed on the photograph, allowing information from the patient chart to be retrieved as text. Other input tools may include hand-held input devices, e.g. touch screens on a portable device such as a phone or tablet, connected to or wirelessly networked with a network or the patient monitoring device. Data selection may occur on a hand-held input device, e.g., on a touch screen on a hand-held input device displaying all or some part of the information on the monitor display.

A doctor can, for example, use an existing device such as a smart phone or tablet to record information about (e.g., take a photograph of or record a dictation regarding) a physiological reminder, a drug label, a data display, a diagnosis, a prescription, a treatment plan, etc. This information can be appended to a time or an analyte reading corresponding to a time that may be recorded and/or displayed by the systems (e.g., bedside monitoring systems) disclosed herein. Thus, the monitoring system and display can provide an ordered repository of historical information, as well as a way to remind a user of that information (or conceal it from some entities, as may be prudent or for compliance with privacy concerns or regulations). The display screen can thus function as a useful visual memory of actions taken or analyses performed not only by the device, but also by a doctor or nurse, for example. Moreover, a patient can also be allowed to annotate data. Different users can have different colors or some personal identification that can be imbedded or otherwise reflected in the saved data or annotation itself. For example, a patient can annotate a data stream to indicate how they felt, how much pain, how and what had been consumed (or eliminated), etc. This disclosure and these principles are not limited to the medical context, but have application in other areas as well.

### NOTES REGARDING INTERPRETATION OF THIS DISCLOSURE

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment. Accordingly, no feature or group of features is necessary or indispensable to each embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose physical computing systems. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device (e.g., hard disks, RAM, ROM, flash memory, etc.). Computer-readable media include non-transitory computer-readable media such as magnetic storage, optical storage (e.g., CD-ROMs or DVDs), semiconductor storage, etc. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above.

1. (canceled)

2. (canceled)

3. (canceled)

4. (canceled)

5. A critical care bedside system for periodic blood withdrawal and automated measurement and display of multiple analyte values, the system comprising:
a connector configured to interface with one lumen of a catheter having constant access to a critical care patient's blood;
a housing configured for bedside placement in the critical care environment, the housing supporting a large touchscreen on its exterior, the large touchscreen covering more than half of a user-facing surface of the housing and configured to provide control and display functions in the critical care environment by showing critical data large enough to be readable to a distance of 30 feet, the housing comprising:
a series of interconnected medical-grade fluid tubes with mechanical valves positioned at intersections between tubes and configured to control fluid flow therein, at least one of said tubes extending out of the housing and configured to connect to the catheter through the connector;
at least two valve control motors configured to interface physically with the valves;
a blood withdrawal pump in fluid communication with the connector and configured to automatically and periodically draw blood through at least one fluid tube and one mechanical valve;
at least one external sensor adjacent to a fluid tube and configured to receive signals related to the presence of fluid within the fluid tubes inside the housing;
at least one analyte measurement complex comprising a flow cell in fluid communication with the fluid tubes and an analysis module, the analysis module configured to interface with the fluid and evaluate resulting signals to determine measurements of at least two analytes;
at least one internal sensor positioned for periodic fluid communication with the blood withdrawal pump, with fluid in a fluid tube in the housing, and with fluid in the connector, the internal sensor sensitive to flow or pressure of fluid therein; and
a computer processor configured to receive signals from the at least one external sensor, the analyte measurement complex, the at least one internal sensor, and the large touchscreen;
the large touchscreen comprising:
a pause button integrated into the touchscreen and operable to cause the computer processor to pause a function of the bedside system, the function comprising one or more of analysis by the analyte measurement complex, fluid flow caused by the withdrawal pump, or display of information on the large touchscreen itself;
a delay button integrated into the touchscreen and operable to cause the computer processor to send a signal to the blood withdrawal pump, thereby deferring automatic operation of the blood withdrawal pump for a time and delaying subsequent fluid flow through the connector and the fluid tubes;
an operating graphic displayable on the large touchscreen, the touchscreen controllable by the processor to display the operating graphic to indicate a running status when the processor determines from sensor signals that the connector is connected with the lumen of the catheter and that normal fluid flow is occurring in the housing through the tubes;
a time graphic displayable on the large touchscreen, the touchscreen controllable by the processor to display the operating graphic to constantly indicate the present time;
at least two prominent current measurement regions of the large touchscreen configured to automatically display by default the most recent analyte value resulting from signals from the analyte measurement complex, said analyte values visible from 30 feet to facilitate emergency action in critical care environments;
an automatically scrolling interactive history region of the large touchscreen that coordinates with the processor to:
receive and display data from the computer processor to record a series of outputs of the analyte measurement complex associated with analyte measurements of the critical care patient;
respond to a touch by a user on a portion of the interactive history region corresponding to a selected time by displaying an annotation interface associated with the selected time, the annotation interface comprising a virtual keyboard display;
accept annotation inputs within the annotation interface, associate the annotation inputs with at least the selected time and data associated with the selected time, and record them in a database;
display a symbol on any future renderings of the interactive history region that include the selected time to indicate presence of an annotation for that selected time; and
facilitate future on-demand retrieval and display of the recorded annotation inputs using the symbol, the on-demand retrieval comprising display of at least the annotation and the data associated with the selected time;
the two prominent current measurement regions of the touchscreen configured to vary a graphic characteristic based on a current value for a corresponding analyte or a predicted value for the corresponding analyte at a future time; and
the automatically scrolling interactive history region configured to coordinate with the processor further to display:
a first line corresponding to an upper threshold for at least one analyte;
a second line corresponding to a lower threshold for the at least one analyte;
a first area under one or more determined concentrations that are between the upper threshold and the lower threshold with a first color; and
a second area under the series of outputs of the analyte measurement complex that are above the upper threshold or below the lower threshold with a second color that contrasts with the first color.

6. The bedside system of claim 5, further comprising a treatment pump configured to deliver an infusion substance to provide glycemic control, wherein the treatment pump is configured to deliver the infusion substance to the patient through the connector.

7. The bedside system of claim 5, wherein the analysis module comprises a spectroscopic analyzer.

8. The bedside system of claim 5, wherein the analysis module comprises an electro-chemical analyzer.

9. The bedside system of claim 5, wherein said at least two analytes comprise glucose.

10. The bedside system of claim 6, wherein the infusion substance comprises a substance selected from the group consisting of insulin and dextrose.

11. The bedside system of claim 5, wherein the blood withdrawal pump is selected from a group consisting of: volumetric pump, syringe pump, peristaltic pump, vacuum pump, electrical pump, mechanical pump, or hydraulic pump.

12. The bedside system of claim 5, wherein a foreground or a background color of one of the two prominent current measurement regions is yellow or orange if the predicted value for concentration of the corresponding analyte at a future time is above a first threshold value or below a second threshold value.

13. The bedside system of claim 5, wherein one of the two prominent current measurement regions is configured to flash or blink if the predicted value for concentration of the corresponding analyte at a future time is above a first threshold or below a second threshold value.

14. The bedside system of claim 5, wherein the graphic characteristic of the two prominent current measurement regions is a foreground or background color.

15. The bedside system of claim 5, wherein the predicted value for the corresponding analyte is determined from a rate of change in concentration of the corresponding analyte.

16. The bedside system of claim 15, wherein the rate of change is based on one or more previous measurements of concentration of the corresponding analyte.

17. The bedside system of claim 15, wherein the rate of change depends on a first dose of an infusion substance in a first time interval.

18. The bedside system of claim 17, wherein the rate of change further depends on a second dose of an infusion substance in a second time interval.

19. The bedside system of claim 5, wherein the retrieval of the recorded annotation is facilitated by using audio and/or visual cues.

20. The bedside system of claim 5, wherein the computer processor is configured to provide a suggested dose for an infusion substance based on the predicted value of concentration of the corresponding analyte.

21. The bedside system of claim 5, wherein the predicted value of concentration of one of the at least two analytes at a future time can be determined from a trend in the values of the concentration of the one of the at least two analytes obtained in one or more previous measurements.

22. The bedside system of claim 5, wherein the predicted value of concentration of one of the at least two analytes is calculated on a moving window basis.

23. The bedside system of claim 5, wherein the automatically scrolling interactive history region comprises one or more trend indicators that provide a graphical indication of time history of concentration of an analyte of interest.

24. The bedside system of claim 5, wherein the annotation interface comprises an input screen shown on the touchscreen.

25. The bedside system of claim 24, wherein the input screen is a bubble visually connected to the portion of the interactive history region touched by the user.

26. The bedside system of claim 5, wherein the interactive history region comprises at least one of: a line graph, a bar graph, or a table.

27. The bedside system of claim 5, wherein the touch of the portion of the interactive history region causes the processor to display additional information corresponding to the data associated with the selected time.

28. The bedside system of claim 5 further comprising a dosage history button integrated into the touchscreen and operable to cause the computer processor to show a dosing history window, the dosing history window configured to display a patient dosing history for an analyte of interest.

29. The bedside system of claim 5, wherein the interactive history region can comprise one or more trend indicators, wherein the one or more trend indicators can correspond to one or more types of analytes.

30. The bedside system of claim 5 further comprising a warning graphic displayable on the large touchscreen, the warning graphic configured to change its graphic characteristic based on a current value or a predicted value at a future time for an analyte of interest.

31. The bedside system of claim 5, wherein the portion of the interactive history region corresponding to the recorded annotation is highlighted to indicate presence of the annotation.

32. The bedside system of claim 5, wherein one or more portions of the interactive history region can be selected for annotation.
